SEQUENCE LISTING (111 sequences)

SEQ ID NO: 1 - protein, 4 aa, Mycoplasma pneumoniae
  MISC_FEATURE (1)..(1): X(1): L, V, or I
  MISC_FEATURE (2)..(2): X(2): A, S, T, V, I
  MISC_FEATURE (3)..(3): X(3): G, A, S
XXXC

SEQ ID NO: 2 - protein, 24 aa, Staphylococcus aureus
MKKIYSFLAGIAAIILVLWGIATH

SEQ ID NO: 3 - protein, 24 aa, Mycoplasma pneumoniae
MKFKLFLGSSFFGVATLLIACGTG

SEQ ID NO: 4 - protein, 30 aa, Mycoplasma pneumoniae
MFGKGLVKKSLLFFSGVSTMAVFLVSCGAT

SEQ ID NO: 5 - protein, 24 aa, Mycoplasma pneumoniae
MKFKLFLGSSFFGVATLLIACGTG

SEQ ID NO: 6 - protein, 25 aa, Mycoplasma pneumoniae
MKYKTVKSIPLFLLGSIVFTACSTP

SEQ ID NO: 7 - protein, 31 aa, Mycoplasma pneumoniae
MKKFLRKPQFWLLTLGGFLSTSVILAACATP

SEQ ID NO: 8 - protein, 31 aa, Mycoplasma pneumoniae
MHTFTRKVKWPFMFTAIGLTFGIVAVACAQP

SEQ ID NO: 9 - protein, 28 aa, Mycoplasma pneumoniae
MKFQRKYWGLLSTLGVSSAVALSACAAQ

SEQ ID NO: 10 - protein, 30 aa, Mycoplasma pneumoniae
MGYKLKRWPLVAFTFTGIGLGVVLAACSAL

SEQ ID NO: 11 - protein, 33 aa, Mycoplasma pneumoniae
MKKWFSSISKKKVSFSTLLLLGSGIVLSSCSNI

SEQ ID NO: 12 - protein, 38 aa, Mycoplasma pneumoniae
MKLSAIISLSVAGTVGTTAVVVPTTITLVNKTHQVEHE

SEQ ID NO: 13 - protein, 37 aa, Mycoplasma pneumoniae
MIFSISKRKLICGFSLVALTIAGIVGGVYLVTKNNQQ

SEQ ID NO: 14 - protein, 37 aa, Mycoplasma pneumoniae
MKKRTKYRYWLNSLAFFGCGVSVGAFFTLFLMGTQSD

SEQ ID NO: 15 - protein, 34 aa, Mycoplasma pneumoniae
MRKKRLLSRISFSSLFLLCGTVLSAYTGIQADLR

SEQ ID NO: 16 - protein, 32 aa, Mycoplasma pneumoniae
MKQKIIGLTLAFFVLFLTAVAILFTVKVQRYL

SEQ ID NO: 17 - protein, 33 aa, Mycoplasma pneumoniae
MMTVEVFSVIILVLLFLILALTLLFVLLNKRTR

SEQ ID NO: 18 - protein, 34 aa, Mycoplasma pneumoniae
MKVLLWIGYVLSFGLLYLYLVKRAKRAALQLNNK

SEQ ID NO: 19 - protein, 47 aa, Mycoplasma pneumoniae
SAKLTLESIAKTFAETSIFAILFLIIVILNLGLLVFLAYQYRVYKKK

SEQ ID NO: 20 - protein, 38 aa, Mycoplasma pneumoniae
MVVIAALLGSLAVLAFLFYLWYLTIFIIVHKNQQYLEQ

SEQ ID NO: 21 - protein, 33 aa, Mycoplasma pneumoniae
MKSLKVALIGSGAVGTSFLYAAMSRGLASEYMV

SEQ ID NO: 22 - DNA, 20 bp, Mus musculus
cccactaacatcaaatgggg

SEQ ID NO: 23 - DNA, 20 bp, Mus musculus
ccttccacaatgccaaagtt

SEQ ID NO: 24 - DNA, 18 bp, Mus musculus
aggcactcccccaaaaga

SEQ ID NO: 25 - protein, 20 aa, Mus musculus
GCTCCTCCACTTGGTGGTTT

SEQ ID NO: 26 - DNA, 20 bp, Mus musculus
gacagactgctctgatggca

SEQ ID NO: 27 - DNA, 20 bp, Mus musculus
tgcacttcttttcgcacaac

SEQ ID NO: 28 - DNA, 19 bp, Mus musculus
ctggcaaaaggatggtgac

SEQ ID NO: 29 - DNA, 20 bp, Mus musculus
gctgatggcctgattgtctt

SEQ ID NO: 30 - DNA, 19 bp, Mus musculus
cttctgggcctgctgttca

SEQ ID NO: 31 - DNA, 21 bp, Mus musculus
ccagcctactcattgggatca

SEQ ID NO: 32 - DNA, 21 bp, Mus musculus
cccagccaggtgtcattttcc

SEQ ID NO: 33 - DNA, 21 bp, Mus musculus
gcattcagttccaggtcagtg

SEQ ID NO: 34 - DNA, 21 bp, Mus musculus
ctcctgaagctgttgcgttac

SEQ ID NO: 35 - DNA, 21 bp, Mus musculus
gctcccttacaggctgagttc

SEQ ID NO: 36 - DNA, 20 bp, Mus musculus
gctgcttccaaacctttgac

SEQ ID NO: 37 - DNA, 20 bp, Mus musculus
agcttctccacagccacaat

SEQ ID NO: 38 - DNA, 20 bp, Mus musculus
gcaacgaagaacaccacaga

SEQ ID NO: 39 - DNA, 20 bp, Mus musculus
atcgaaaagcccgaaagagt

SEQ ID NO: 40 - DNA, 20 bp, Mus musculus
tgatgcacttgcagaaaaca

SEQ ID NO: 41 - DNA, 21 bp, Mus musculus
ggtcttggtccttagccactc

SEQ ID NO: 42 - DNA, 20 bp, Mus musculus
ccaaggtcagcgttccaaca

SEQ ID NO: 43 - DNA, 22 bp, Mus musculus
agaggaggtagcgtgattgaca

SEQ ID NO: 44 - DNA, 21 bp, Mus musculus
cagcaccagcttcttcatcag

SEQ ID NO: 45 - DNA, 22 bp, Mus musculus
tcttcaaaggcttcatctgcaa

SEQ ID NO: 46 - DNA, 22 bp, Mus musculus
actgtacaaccgcagtaatacg

SEQ ID NO: 47 - DNA, 23 bp, Mus musculus
agtgaacattacagatttatccc

SEQ ID NO: 48 - DNA, 21 bp, Mus musculus
tgctggattgcagagcagtaa

SEQ ID NO: 49 - DNA, 21 bp, Mus musculus
gcatgcagagattccgagaga

SEQ ID NO: 50 - DNA, 20 bp, Mycoplasma pneumoniae
acgatgattacaggcggttc

SEQ ID NO: 51 - DNA, 20 bp, Mycoplasma pneumoniae
gttggtggcctcttgttgat

SEQ ID NO: 52 - DNA, 19 bp, Mycoplasma pneumoniae
tcccagcaagtgtgaaccc

SEQ ID NO: 53 - DNA, 19 bp, Mycoplasma pneumoniae
gttttcgctcatcaggtcg

SEQ ID NO: 54 - DNA, 20 bp, Mycoplasma pneumoniae
caaaaacaaggaccccgtcg

SEQ ID NO: 55 - DNA, 20 bp, Mycoplasma pneumoniae
cattcaacccaaaccaaagc

SEQ ID NO: 56 - DNA, 20 bp, Mycoplasma pneumoniae
atttgtctaagcgagcttcc

SEQ ID NO: 57 - DNA, 20 bp, Mycoplasma pneumoniae
ttaatctggtaagccattcg

SEQ ID NO: 58 - DNA, 825 bp, Mycoplasma pneumoniae
atgaagttaccacctcgaagaaagcttaaactgtttttattagcctgaatgctagtgctgttcagcgctttaatagtgcttgcaaccttaattttggtacagcacaacaataccgaactgacagaagttaagagtgaattgagtccccttaacgttgttttacacgcagaagaggatacagtacaaattcagggcaagccgattactgagcaagcatggtttattcctacagttgctggttgctttggttttagtgccctagccatcatcttgggccttgctataggactgccaattgtgaagcgcaaggaaaaacgcttattggaggaaaaggaacgccaagaacagttagcggaacagctacaacgcatttctgcccaacaagaagagcaacaagcgttagaacaacaagcagctgctgaagcccatgctgaagcggaagttgaaccagcaccacaaccagtaccagtaccacctcaaccccaagtccaaattaacttcggtccccgtactggtttcccacctcaacccggtatggcgcctcgtccaggtatgccgccacaccccggtatggctccaagacctggtttcccacctcaacccggtatggcgcctcgtccaggtatgccgccacaccccggtatggctccaagacctggtttcccacctcaacccggtatggcgcctcgtccaggcatgccgccacaccccggtatggctccaagacctggtttcccacctcaacccggtatggcgcctcgtcccggaatgcaaccaccacgtcctggcatgccaccccaacccggttttccaccaaaacgctaa

SEQ ID NO: 59 - protein, 274 aa, Mycoplasma pneumoniae
MKLPPRRKLKLFLLAWMLVLFSALIVLATLILVQHNNTELTEVKSELSPLNVVLHAEEDTVQIQGKPITEQAWFIPTVAGCFGFSALAIILGLAIGLPIVKRKEKRLLEEKERQEQLAEQLQRISAQQEEQQALEQQAAAEAHAEAEVEPAPQPVPVPPQPQVQINFGPRTGFPPQPGMAPRPGMPPHPGMAPRPGFPPQPGMAPRPGMPPHPGMAPRPGFPPQPGMAPRPGMPPHPGMAPRPGFPPQPGMAPRPGMQPPRPGMPPQPGFPPKR

SEQ ID NO: 60 - DNA, 3657 bp, Mycoplasma pneumoniae
atgaaatcgaagctaaagttaaaacgttatttactgtttttaccacttttaccgctagggacgttgtcactagccaacacctacctcctccaagaccacaacaccctcaccccctacacgccctttacgacaccgctcaatggggggctggatgtcgtgcgcgccgcccatttacacccctcatacgaactcgtggactgaaagcgggtgggggataccaagttggtggcgctggtccgctcagcgttggtcagggtgaaattccaggacacaacgagttcggatcaaagtaataccaaccaaaatgccttgagttttgatacccaagaatcacagaaggcacttaatggctcgcagagtggatcttctgacacttccgggtctaactcccaagacttcgccagctatgtcctcatctttaaagccgcgcccagggccacgtgggtgtttgaacgcaagattaagttggcgttgccctacgttaagcaggaaagtcagggttccggcgatcaaggttccaatggtaagggctccctctacaaaaccctccaagacctcctcgtcgaacaacccgtgaccccttacaccccgaatgcggggttagcccgggtgaatggggttgctcaggatacggttcattttggttcgggtcaagaatcgagttggaattcccaacgttcccaaaaaggccttaaaaacaaccccggacccaaagccgtcaccggctttaagctcgataagggccgcgcgtaccggaagctgaatgaaagttgaccggtgtatgaacccctggattcgaccaaggaggggaaggggaaggatgagagctcttggaaaaattcggaaaaaacaacagcggaaaatgatgccccgttggtggggatggttggaagtggtgcggctggaagtgcttctagtttacaaggcaatggctcgaacagttcggggttaaaatcgctcttgagatcagcacctgtcagtgttccaccaagcagtacaagtaatcaaactttaagcttatctaaccccgctcctgtgggcccacaagcggttgtaagccaacccgcggggggtgctacggcagcagtgtccgtcaatcgcacagcgagtgacaccgccacctttagcaagtacctcaacaccgcccaggccttgcaccagatgggggtgattgttccggggttggaaaaatgaggtggtaacaacggtacgggtgtagtggctagccgacaggatgctacttccactaacctgccccatgcggcaggtgcttcccaaacgggtttgggaactggttcgccccgcgaaccagctttaaccgcaacgtcacagcgtgccgtcacggtggttgctggcccccttcgtgcgggcaatagcagtgaaactgatgccctaccgaatgtcatcacccagctctatcatacttcaaccgcccaactcgcttacttaaatggccagatcgttgtgatgggttccgaccgggtaccgagtctttggtattgagttgtcggggaggaccaggaatcgggcaaagcgacctgatgagcgaaaaccgagctcaactggggcaccgacaagcagaagcagtttgtcgaaaaccagttggggtttaaagatgactcaaattcggattccaaaaattcgaatttgaaggcccaaggcctcacccaacccgcctacctcatcgccggtcttgacgttgtggccgaccacctcgtctttgcggcctttaaagcgggcgcggtggggtatgatatgacgactgattcgagcgcttcgacctacaaccaagcactcgcctggtcgaccacggccgggttggacagtgatggggggtacaaggccttggtggaaaacacggccgggctcaacggcccgattaatggcttgtttaccctgctcgacacctttgcgtatgtgacccccgtgagtgggatgaaaggggggagtcagaataatgaagaagtgcaaacgacttacccggtcaagtccgaccaaaaggccaccgccaaaattgcctccttaattaatgccagcccactcaacagttatggggatgatggggtgaccgtgtttgatgccctgggccttaactttaactttaagttgaacgaggagcgcttgccatcgcgcaccgaccaactgcttgtgtatgggattgtaaacgaaagtgaactgaagtccgcacgggaaaatgcccagtcgacctccgatgataattcaaacaccaaagtcaagtgaaccaacaccgcctcgcactacctccccgtgccgtattactacagtgccaatttccccgaagcgggtaacagaaggcgagcggagcagcggaatggggtgaagattagcaccttggaatcgcaagccactgatggctttgccaactcgttacttaactttggtaccggtcttaaagccggtgttgacccagctccagtagcacggggtcataaaccgaactatagtgcagtactactagtgcgtggtggcgttgtaaggttaaactttaaccccgatactgataaactgttggattctactgacaaaaacagtgaacctatctccttctcctataccccatttgggtctgctgaaagtgccgtagacctcaccacgttgaaggatgtgacctatattgctgaaagtggtctgtggttctatacctttgacaatggtgaaaaaccaacgtacgatggtaaacaacaacaggtcaaaaaccgcaagggttatgctgtgattaccgtatcacgtaccggaattgaatttaacgaggacgctaataccacaaccttaagccaagccccagctgctttggctgtccaaaacgggattgcttccagtcaggacgacctcacaggcatcctaccgttatccgatgagttctccgctgtgattaccaaggatcaaacatggaccggtaaggttgatatctataagaacaccaacgggttgtttgaaaaggatgatcagctatcggaaaacgtgaagaggcgtgacaacggtttggtccctatttacaacgaaggtatcgtcgatatttggggcagagtggattttgctgccaacagtgttttgcaagcgcgtaacctcactgataaaacggttgatgaggtgatcaataaccccgatatcctccaaagcttctttaagtttaccccagcctttgataaccaaagagcaatgctagtgggggaaaagacatcggatactaccttaacggttaaaccgaagattgagtacttggatggtaacttctatggtgaggattccaagattgctggaattccgctcaacattgatttcccttcccggatttttgctggctttgctgctttaccgtcctgggtcattccggtatcagtcggttcatcggtgggcattctcttaatcctgctcatcttaggccttggtattggaattccaatgtataaggtccgcaagcttcaagactccagctttgttgatgtgtttaaaaaggtggatacgttgacaaccgctgtgggtagcgtgtacaagaagattatcacccaaacgagtgtgatcaaaaaagctcctagtgcgttgaaagctgctaataacgctgctcctaaagcaccagttaaaccagctgctccaacagctccaagaccaccagtccaaccacctaaaaaggcttaa

SEQ ID NO: 61 - protein, 1218 aa, Mycoplasma pneumoniae
MKSKLKLKRYLLFLPLLPLGTLSLANTYLLQDHNTLTPYTPFTTPLNGGLDVVRAAHLHPSYELVDWKRVGDTKLVALVRSALVRVKFQDTTSSDQSNTNQNALSFDTQESQKALNGSQSGSSDTSGSNSQDFASYVLIFKAAPRATWVFERKIKLALPYVKQESQGSGDQGSNGKGSLYKTLQDLLVEQPVTPYTPNAGLARVNGVAQDTVHFGSGQESSWNSQRSQKGLKNNPGPKAVTGFKLDKGRAYRKLNESWPVYEPLDSTKEGKGKDESSWKNSEKTTAENDAPLVGMVGSGAAGSASSLQGNGSNSSGLKSLLRSAPVSVPPSSTSNQTLSLSNPAPVGPQAVVSQPAGGATAAVSVNRTASDTATFSKYLNTAQALHQMGVIVPGLEKWGGNNGTGVVASRQDATSTNLPHAAGASQTGLGTGSPREPALTATSQRAVTVVAGPLRAGNSSETDALPNVITQLYHTSTAQLAYLNGQIVVMGSDRVPSLWYWVVGEDQESGKATWWAKTELNWGTDKQKQFVENQLGFKDDSNSDSKNSNLKAQGLTQPAYLIAGLDVVADHLVFAAFKAGAVGYDMTTDSSASTYNQALAWSTTAGLDSDGGYKALVENTAGLNGPINGLFTLLDTFAYVTPVSGMKGGSQNNEEVQTTYPVKSDQKATAKIASLINASPLNSYGDDGVTVFDALGLNFNFKLNEERLPSRTDQLLVYGIVNESELKSARENAQSTSDDNSNTKVKWTNTASHYLPVPYYYSANFPEAGNRRRAEQRNGVKISTLESQATDGFANSLLNFGTGLKAGVDPAPVARGHKPNYSAVLLVRGGVVRLNFNPDTDKLLDSTDKNSEPISFSYTPFGSAESAVDLTTLKDVTYIAESGLWFYTFDNGEKPTYDGKQQQVKNRKGYAVITVSRTGIEFNEDANTTTLSQAPAALAVQNGIASSQDDLTGILPLSDEFSAVITKDQTWTGKVDIYKNTNGLFEKDDQLSENVKRRDNGLVPIYNEGIVDIWGRVDFAANSVLQARNLTDKTVDEVINNPDILQSFFKFTPAFDNQRAMLVGEKTSDTTLTVKPKIEYLDGNFYGEDSKIAGIPLNIDFPSRIFAGFAALPSWVIPVSVGSSVGILLILLILGLGIGIPMYKVRKLQDSSFVDVFKKVDTLTTAVGSVYKKIITQTSVIKKAPSALKAANNAAPKAPVKPAAPTAPRPPVQPPKKA

SEQ ID NO: 62 - DNA, 717 bp, Artificial Sequence
  P3 promoter + p30 signal sequence + SH3 domain + Flag tag
tattgcataatattgtataatattgtataattaaaaataatgaagttaccacctcgaagaaagcttaaactgtttttattagcctgaatgctagtgctgttcagcgctttaatagtgcttgcaaccttaattttggtacagcacaacaataccgaactgacagaagttaagagtgaattgagtccccttaacgttgttttacacgcagaagaggatacagtacaaattcagggcaagccgattactgagcaagcatggtttattcctacagttgctggttgctttggttttagtgccctagccatcatcttgggccttgctataggactgccaattgtgaagcgcaaggaaaaacgcttattggaggaaaaggaacgccaagaacagttagcggaacagctctcctgaaagaaaaaccaatacggtacctggtacaaacccgaaaacgccacctttgtgaacggtaaccaaccaattgttacccgcattggtagtccctttttaaacgctccagttggtggtaacctccccgccggtgctaccattgtgtacgatgaagtttgtattcaagcgggtcacatttggattggttacaacgcctacaacggtaaccgcgtgtactgcccagttcgtacctgtcaaggggtgcctccaaatcaaattcctggcgttgcgtggggtgtttttaaggattacaaggatgacgacgataagtaa

SEQ ID NO: 63 - protein, 105 aa, Artificial Sequence
  SH3 domain + Flag tag
LSWKKNQYGTWYKPENATFVNGNQPIVTRIGSPFLNAPVGGNLPAGATIVYDEVCIQAGHIWIGYNAYNGNRVYCPVRTCQGVPPNQIPGVAWGVFKDYKDDDDK

SEQ ID NO: 64 - DNA, 1041 bp, Artificial Sequence
  P3 promoter + P30 signal sequence + SH3 chimera + Flag tag
tattgcataatattgtataatattgtataattaaaaataatgaagttaccacctcgaagaaagcttaaactgtttttattagcctgaatgctagtgctgttcagcgctttaatagtgcttgcaaccttaattttggtacagcacaacaataccgaactgacagaagttaagagtgaattgagtccccttaacgttgttttacacgcagaagaggatacagtacaaattcagggcaagccgattactgagcaagcatggtttattcctacagttgctggttgctttggttttagtgccctagccatcatcttgggccttgctataggactgccaattgtgaagcgcaaggaaaaacgcttattggaggaaaaggaacgccaagaacagttagcggaacagctctcctgaaagaaaaaccaatacggtacctggtacaaacccgaaaacgccacctttgtgaacggtaaccaaccaattgttacccgcattggtagtccctttttaaacgctccagttggtggtaacctccccgccggtgctaccattgtgtacgatgaagtttgtattcaagcgggtcacatttggattggttacaacgcctacaacggtaaccgcgtgtactgcccagttcgtacctgtcaaggggtgcctccaaatcaaattcctggcgttgcgtggggtgtttttaagggtggtgaaaatttatactttcaaggtggtggtctctcctgaaagaaaaaccaatacggtacctggtacaaacccgaaaacgccacctttgtgaacggtaaccaaccaattgttacccgcattggtagtccctttttaaacgctccagttggtggtaacctccccgccggtgctaccattgtgtacgatgaagtttgtattcaagcgggtcacatttggattggttacaacgcctacaacggtaaccgcgtgtactgcccagttcgtacctgtcaaggggtgcctccaaatcaaattcctggcgttgcgtggggtgtttttaaggattacaaggatgacgacgataagtaa

SEQ ID NO: 65 - protein, 333 aa, Artificial Sequence
  SH3 chimera + Flag tag
MKLPPRRKLKLFLLAWMLVLFSALIVLATLILVQHNNTELTEVKSELSPLNVVLHAEEDTVQIQGKPITEQAWFIPTVAGCFGFSALAIILGLAIGLPIVKRKEKRLLEEKERQEQLAEQLSWKKNQYGTWYKPENATFVNGNQPIVTRIGSPFLNAPVGGNLPAGATIVYDEVCIQAGHIWIGYNAYNGNRVYCPVRTCQGVPPNQIPGVAWGVFKGGENLYFQGGGLSWKKNQYGTWYKPENATFVNGNQPIVTRIGSPFLNAPVGGNLPAGATIVYDEVCIQAGHIWIGYNAYNGNRVYCPVRTCQGVPPNQIPGVAWGVFKDYKDDDDK

SEQ ID NO: 66 - DNA, 813 bp, Artificial Sequence
  P3 promoter + P30 signal sequence + Nanobody
tattgcataatattgtataatattgtataattaaaaataatgaagttaccacctcgaagaaagcttaaactgtttttattagcctgaatgctagtgctgttcagcgctttaatagtgcttgcaaccttaattttggtacagcacaacaataccgaactgacagaagttaagagtgaattgagtccccttaacgttgttttacacgcagaagaggatacagtacaaattcagggcaagccgattactgagcaagcatggtttattcctacagttgctggttgctttggttttagtgccctagccatcatcttgggccttgctataggactgccaattgtgaagcgcaaggaaaaacgcttattggaggaaaaggaacgccaagaacagttagcggaacaggcgcaggtgcagctgcaggaaagcggcggcggcctggtgcaggcgggcggcagcctgaccctgagctgcgcggcgagcggccgcacctttagcaactatgcgatgggctggtttcgccaggcgccgggcaaagaacgcgaatttgtggcgatgattagctggaacggcgaaaacacctattatgcggatagcgtgaaaggccgctttaccattagccgcgataacgcgaaaaacaccgtgtatctgcagatgaacagcctgaaaccggaagataccgcggtgtattattgcgcggtgcgcattctgagcggctggtatgatcgcccggatgaatatggctattggggccagggcacccaggtgaccgtgagcagcgcggcggcgcatcaccatcaccatcacgcggcggcgtaa

SEQ ID NO: 67 - protein, 257 aa, Artificial Sequence
  Nanobody
MKLPPRRKLKLFLLAWMLVLFSALIVLATLILVQHNNTELTEVKSELSPLNVVLHAEEDTVQIQGKPITEQAWFIPTVAGCFGFSALAIILGLAIGLPIVKRKEKRLLEEKERQEQLAEQAQVQLQESGGGLVQAGGSLTLSCAASGRTFSNYAMGWFRQAPGKEREFVAMISWNGENTYYADSVKGRFTISRDNAKNTVYLQMNSLKPEDTAVYYCAVRILSGWYDRPDEYGYWGQGTQVTVSSAAAHHHHHHAAA

SEQ ID NO: 68 - DNA, 1284 bp, Artificial Sequence
  P30 signal sequence + Vsa
atgaagttaccacctcgaagaaagcttaaactgtttttattagcctgaatgctagtgctgttcagcgctttaatagtgcttgcaaccttaattttggtacagcacaacaataccgaactgacagaagttaagagtgaattgagtccccttaacgttgttttacacgcagaagaggatacagtacaaattcagggcaagccgattactgagcaagcatggtttattcctacagttgctggttgctttggttttagtgccctagccatcatcttgggccttgctataggactgccaattgtgaagcgcaaggaaaaacgcttattggaggaaaaggaacgccaagaacagttagcggaacagcagaagcaggacgacggggctattaagaaggacctcgaatccaagattgatgtgttgaccttctttcccaagagtgttgatgaaaccgcgcaatgaaccgtgagtcgcttactcagtgaataccgcaccgctaagagttccgatagtaacttgacctttgacgcgtttgttaagagtaaactcggtagtaaattgggtagtgattacgaactcacctttaaggaaaaaggtgccgacaagatcgaatacaccatctacaagaaaagtaacaaggctgtgagttccaccaaagttttagaagataccaagattggtcaaagtgacgcccaaaaagtgaacgcttgattagatcgcgtgaagagtctcgttgccaacagttccgacctcaagggtaagaaacccagtgaagtgatcgaaatcgaaaccagtgatgttaagtttaaagaaacccgcgatggtcaagacgtggaatttgacgttcccaccaactggggtaccgttattaccaaaagttccgcgaacgatgctgcgaacgacgccgaaggtaccctcaaggtgagtgttcaatttagttccggtgattttcgtcgcagtaaagacttgaacttaaccggtttgaagaccagtaacaacgaacaaggtggtaacaacagtggttccaacggtagtaaacccatgaccccacccaccaccggtagtggttccggtagtaccgataccaagccacaaaccccacccaccaccggctccggtagtggttccaccgacaccaaaccccagaccccacccactactgggtccggcagtggctccaccgacaccaaagcgcagaccccacccactacagggagtggctccggcagtaccgataccaaagcgcaaaccccacccactactggctccggcagtggcagtatcaactag

SEQ ID NO: 69 - protein, 427 aa, Mycoplasma pulmonis
MKLPPRRKLKLFLLAWMLVLFSALIVLATLILVQHNNTELTEVKSELSPLNVVLHAEEDTVQIQGKPITEQAWFIPTVAGCFGFSALAIILGLAIGLPIVKRKEKRLLEEKERQEQLAEQQKQDDGAIKKDLESKIDVLTFFPKSVDETAQWTVSRLLSEYRTAKSSDSNLTFDAFVKSKLGSKLGSDYELTFKEKGADKIEYTIYKKSNKAVSSTKVLEDTKIGQSDAQKVNAWLDRVKSLVANSSDLKGKKPSEVIEIETSDVKFKETRDGQDVEFDVPTNWGTVITKSSANDAANDAEGTLKVSVQFSSGDFRRSKDLNLTGLKTSNNEQGGNNSGSNGSKPMTPPTTGSGSGSTDTKPQTPPTTGSGSGSTDTKPQTPPTTGSGSGSTDTKAQTPPTTGSGSGSTDTKAQTPPTTGSGSGSIN

SEQ ID NO: 70 - DNA, 114 bp, Artificial Sequence
  Oligo to delete lipoprotein MPN489
ttgacactcgagtccttggtgagtggtgcggctttggctttaccgttcgtatagcatacattatacgaagttatattgggcagaccagcattagattgctatggtatatttaag

SEQ ID NO: 71 - DNA, 4100 bp, Mycoplasma pneumoniae
atgggttataagttaaagcgatgacctttagttgcgtttacatttaccggaattggcctaggggtggtgctagcggcgtgttctgcactcaatacctccaatctgtttccccgtcaaaaccgctccaagcagttgattgggtttaccgaaaacaacatcattaaacctgaagctgtactaaaagccgctttagctgaggacaatggtacggaaaccattttaagggttaactttggtgaagcgttaaagagttggtaccaaaacaataaggatcgcaacattgctacccgtttaactatctttagtgaaaacgtggaggatgaacatgataacctgttggaccaaaaacaacaggccgaaccgattaattgaccaatagagctccaaaaggaatacgaccagtggggtgggagtgaaagttcctgaaaggccttaaagttatacgaccgtttaatagccgacttccaatcacttatctttagcaacattgttgctaatgtgcagttgactgatggtagtgatcagtttaaacccactaccaaagataacctcgacagtactagcaacaaaatcaagtttgttaattccaaaccaaatgatcctaacggggagttctttgctaacctccaggcttatctatttgcccagtgagtagtggaggaaaaccccctccccctcacccaagccttcttcgcgtaccaagcacccaaggacgggctcgacagcttatacgaccaagccgcgattggtagtgccttacagttgggctatgccttccccgcgtttcgcgagcccaataatggacaaagccaaggtaaaaccaccttcgaccccacccccaacagtgcccaaaacttcggggatttcattaaggcggtgtttccggagcagaagaatggtcaaacccaacaatccaatacatcctcccgcaccgggttgtttgactggcaaaccaagtgaaacaccaatggcgctgccaataagctcttggtgaccaagtccaacctgcgcggggcctttaagggcgtggggttagctacggccattattgaccagtacgagtacctggtgggtggaagtaaaacgagttcgttgcccgaggtcaaggtagattcgaataagtccaaccaaaatccgctggatagtttttttatggaggggaaggatgcggtggcgattagatccatagtttcgcgtgcgaaaattgccatgaccgaccaaacccccgggtttaaggtcaacccggcgtttgtcaaggtgaagcagagtcagcagaacgataccttttaccaaaaccagcgcaaactgagcggcgggcaaagtggggataataactcccagggtaaacaccactacctccaagacgcggtgcggttgacgagtagtcaagcaatggcggcagcttctactggggcagattctagtagtggtaccaatgttggtggtagctccgggggcaactctgttttaattccgctcccaaggagtgccgcccttacccacacccagcaacaagttcaacaaaccacctccacactccaaacccctgtttacgcccgtggtgacgatggtacatacgccctggcaattgacggtggtgattattttttggcgaacaacaaacgcgatttcaccaaacaagccgacatcttgctgtaccgctatttacaagctaagagtaataactttaaggaaaacggcgtcgagtttagtttgaacttactggaatctggcagcctgttccaaacgtgagcacaaacggggttaaccgcgaagttgtacggtgctttggtggcgatgatgggttcgggtcaggggacccaagttaagggcagtgtgcaggggagtagccgagcagcttctgttagcgttcaaactacccagcaaaacagacagcaatcgactgatacccaagaatcggaagtggttaagttagccaagtccctgctgaaatcaagtgcggatttggccaaacccttcactgataaccccacctttaaaaaggccttaaccgacatccaatccgagtacaaggattatttggctgctgcggggaagttaagtgagtttaagaaagatttgggggaggtgagtggcttacagcaagctattattgaccgcgctgataagtacattcaactagaaaaacaagcccaaaagagtgcgattggcttgggccaaccgctcccttaccaacgcgctagtgatggtagttaccccgctttagagaagttctttattccggaagatagtgcagcagatggaaaagtaaaagcatctgaatcaggatctgccgctttagtaacactaaagacaacagatagtcaaaaaagcacaaacaccgttaaacaaccggacatcaaaccgacccgggaaaacaacgacaaaaagttaaagcagttaactagtgatgtagaaactaaagcatctagtttaatcaccaaatgaggtgcaacaccccaaattggtagtcagttctccgaaattgtttcgctcaagagtaaggacaataagccccagaccaacatgatcttggccttattgtctgatgttgggattaagtgaaccaagattctcaattcctttaaggagtggttctttactaataccaacgactttaagaacaattacgattctgaaaagaaagaacttaaaggcaatgagtacaaggactttaacgacctcgttaaacaaaccttgtacttacgttcctgacaacgactcacttccaaggaaaagtttggttattacaaggagttgggtagtgtaaaagcacaagcagctcaatcaggaatggtttcactttcaagtagcgcagcagttgcaaatgctgtagcttcaagtggcatgcaaaaatctggtgatcaaaccctcttggaactcggtaaaaaagcgtttgaaagtgaactggaagcatctagtagtgatgggcagtacaagtacctgcgcttcttgtccaccttaatgtgactagttaaggatggggcgaagaactataagcgcttgctccaacaagcgattaccgtgggtacccgtgcctttgtgtcgtgaacggtgagctatgatgatacggctacggcatcagcagccgcagcaaaagcgcaagtagcagtattaaagacagctcaggcaacaaatactcaaagcgacaaccccttcaataagtttgtccaaaaccccgattatgtccagggttcggaaaccaactgatttaacgataagagtacgccaattaaacccgattcgttactggaaagcgaaagcacttacaactttaccgcggaaccgtttgatgataagacgaaaagtcaaaagaggtctactggtggtaccactaacgaaaaacacttctttggctttaacggtttaacgattaactcgccccaaagtgtgtcgactgctagtgccggactcacggaacaaatctttaacaactttggtcagttagtaacaagtagtgacaaatccggggccttgagccagtacaaggataaagcaaccctgaaacgcttaatccaaaacactaattccgatgccgagctcaatgcctttggtgaagtgttgcaccgtgcagttaatgtcgataccagtaacttaggacgttttaactccagtggggaaccgttgattagctttgacaacaagaagaagttcttggttgatgtagtggacaaactggatgatgtttactttaacaagttcgaaggttatgttggccaaaccaaggtgaagatgtctgattcaagtagtagttcccaaggtacgaagactataagaaagccaaagccgcaccactcaccaaggactcgagtgtcaagactttgagctatgtcattcagattaccaacgaggacgttaacaaaatttctgctagttgaaaaactgataagaacagtgctttaggcctcaaaaaggaaatctttatggctttactagtagaacaagccttggacgctggtacgcaagagttagcctttttagacttagtgcgcgctaaccaggtgcaaaacaaacgtagtcaccagcaaatcctggtgggtgacaaacgcttgtatgatgcgttagggcaaacgctagcgcttagtgcgaaaaatctttaa

SEQ ID NO: 72 - protein, 1366 aa, Mycoplasma pneumoniae
MGYKLKRWPLVAFTFTGIGLGVVLAACSALNTSNLFPRQNRSKQLIGFTENNIIKPEAVLKAALAEDNGTETILRVNFGEALKSWYQNNKDRNIATRLTIFSENVEDEHDNLLDQKQQAEPINWPIELQKEYDQWGGSESSWKALKLYDRLIADFQSLIFSNIVANVQLTDGSDQFKPTTKDNLDSTSNKIKFVNSKPNDPNGEFFANLQAYLFAQWVVEENPLPLTQAFFAYQAPKDGLDSLYDQAAIGSALQLGYAFPAFREPNNGQSQGKTTFDPTPNSAQNFGDFIKAVFPEQKNGQTQQSNTSSRTGLFDWQTKWNTNGAANKLLVTKSNLRGAFKGVGLATAIIDQYEYLVGGSKTSSLPEVKVDSNKSNQNPLDSFFMEGKDAVAIRSIVSRAKIAMTDQTPGFKVNPAFVKVKQSQQNDTFYQNQRKLSGGQSGDNNSQGKHHYLQDAVRLTSSQAMAAASTGADSSSGTNVGGSSGGNSVLIPLPRSAALTHTQQQVQQTTSTLQTPVYARGDDGTYALAIDGGDYFLANNKRDFTKQADILLYRYLQAKSNNFKENGVEFSLNLLESGSLFQTWAQTGLTAKLYGALVAMMGSGQGTQVKGSVQGSSRAASVSVQTTQQNRQQSTDTQESEVVKLAKSLLKSSADLAKPFTDNPTFKKALTDIQSEYKDYLAAAGKLSEFKKDLGEVSGLQQAIIDRADKYIQLEKQAQKSAIGLGQPLPYQRASDGSYPALEKFFIPEDSAADGKVKASESGSAALVTLKTTDSQKSTNTVKQPDIKPTRENNDKKLKQLTSDVETKASSLITKWGATPQIGSQFSEIVSLKSKDNKPQTNMILALLSDVGIKWTKILNSFKEWFFTNTNDFKNNYDSEKKELKGNEYKDFNDLVKQTLYLRSWQRLTSKEKFGYYKELGSVKAQAAQSGMVSLSSSAAVANAVASSGMQKSGDQTLLELGKKAFESELEASSSDGQYKYLRFLSTLMWLVKDGAKNYKRLLQQAITVGTRAFVSWTVSYDDTATASAAAAKAQVAVLKTAQATNTQSDNPFNKFVQNPDYVQGSETNWFNDKSTPIKPDSLLESESTYNFTAEPFDDKTKSQKRSTGGTTNEKHFFGFNGLTINSPQSVSTASAGLTEQIFNNFGQLVTSSDKSGALSQYKDKATLKRLIQNTNSDAELNAFGEVLHRAVNVDTSNLGRFNSSGEPLISFDNKKKFLVDVVDKLDDVYFNKFEGYVGQTKVKMSDSSSSSQGTKTIRKPKPHHSPRTRVSRLWAMSFRLPTRTLTKFLLVEKLIRTVLWASKRKSLWLYWWNKPWTLVRKSWPFWTWCALTRCKTNVVTSKSWWVTNACMMRWGKRWRLVRKIF

SEQ ID NO: 73 - DNA, 4100 bp, Mycoplasma pneumoniae
atgggttataagttaaagcgatgacctttagttgcgtttgtctttgtagcaattgccctagcggtggtgctagcggcgtctaatgcactcaatacctccaatctgtttccccgtcaaaaccgctccaagcagttgattgggtttaccgaaaacaacatcattaaacctgaagctgtactaaaagccgctttagctgaggacaatggtacggaaaccattttaagggttaactttggtgaagcgttaaagagttggtaccaaaacaataaggatcgcaacattgctacccgtttaactatctttagtgaaaacgtggaggatgaacatgataacctgttggaccaaaaacaacaggccgaaccgattaattgaccaatagagctccaaaaggaatacgaccagtggggtgggagtgaaagttcctgaaaggccttaaagttatacgaccgtttaatagccgacttccaatcacttatctttagcaacattgttgctaatgtgcagttgactgatggtagtgatcagtttaaacccactaccaaagataacctcgacagtactagcaacaaaatcaagtttgttaattccaaaccaaatgatcctaacggggagttctttgctaacctccaggcttatctatttgcccagtgagtagtggaggaaaaccccctccccctcacccaagccttcttcgcgtaccaagcacccaaggacgggctcgacagcttatacgaccaagccgcgattggtagtgccttacagttgggctatgccttccccgcgtttcgcgagcccaataatggacaaagccaaggtaaaaccaccttcgaccccacccccaacagtgcccaaaacttcggggatttcattaaggcggtgtttccggagcagaagaatggtcaaacccaacaatccaatacatcctcccgcaccgggttgtttgactggcaaaccaagtgaaacaccaatggcgctgccaataagctcttggtgaccaagtccaacctgcgcggggcctttaagggcgtggggttagctacggccattattgaccagtacgagtacctggtgggtggaagtaaaacgagttcgttgcccgaggtcaaggtagattcgaataagtccaaccaaaatccgctggatagtttttttatggaggggaaggatgcggtggcgattagatccatagtttcgcgtgcgaaaattgccatgaccgaccaaacccccgggtttaaggtcaacccggcgtttgtcaaggtgaagcagagtcagcagaacgataccttttaccaaaaccagcgcaaactgagcggcgggcaaagtggggataataactcccagggtaaacaccactacctccaagacgcggtgcggttgacgagtagtcaagcaatggcggcagcttctactggggcagattctagtagtggtaccaatgttggtggtagctccgggggcaactctgttttaattccgctcccaaggagtgccgcccttacccacacccagcaacaagttcaacaaaccacctccacactccaaacccctgtttacgcccgtggtgacgatggtacatacgccctggcaattgacggtggtgattattttttggcgaacaacaaacgcgatttcaccaaacaagccgacatcttgctgtaccgctatttacaagctaagagtaataactttaaggaaaacggcgtcgagtttagtttgaacttactggaatctggcagcctgttccaaacgtgagcacaaacggggttaaccgcgaagttgtacggtgctttggtggcgatgatgggttcgggtcaggggacccaagttaagggcagtgtgcaggggagtagccgagcagcttctgttagcgttcaaactacccagcaaaacagacagcaatcgactgatacccaagaatcggaagtggttaagttagccaagtccctgctgaaatcaagtgcggatttggccaaacccttcactgataaccccacctttaaaaaggccttaaccgacatccaatccgagtacaaggattatttggctgctgcggggaagttaagtgagtttaagaaagatttgggggaggtgagtggcttacagcaagctattattgaccgcgctgataagtacattcaactagaaaaacaagcccaaaagagtgcgattggcttgggccaaccgctcccttaccaacgcgctagtgatggtagttaccccgctttagagaagttctttattccggaagatagtgcagcagatggaaaagtaaaagcatctgaatcaggatctgccgctttagtaacactaaagacaacagatagtcaaaaaagcacaaacaccgttaaacaaccggacatcaaaccgacccgggaaaacaacgacaaaaagttaaagcagttaactagtgatgtagaaactaaagcatctagtttaatcaccaaatgaggtgcaacaccccaaattggtagtcagttctccgaaattgtttcgctcaagagtaaggacaataagccccagaccaacatgatcttggccttattgtctgatgttgggattaagtgaaccaagattctcaattcctttaaggagtggttctttactaataccaacgactttaagaacaattacgattctgaaaagaaagaacttaaaggcaatgagtacaaggactttaacgacctcgttaaacaaaccttgtacttacgttcctgacaacgactcacttccaaggaaaagtttggttattacaaggagttgggtagtgtaaaagcacaagcagctcaatcaggaatggtttcactttcaagtagcgcagcagttgcaaatgctgtagcttcaagtggcatgcaaaaatctggtgatcaaaccctcttggaactcggtaaaaaagcgtttgaaagtgaactggaagcatctagtagtgatgggcagtacaagtacctgcgcttcttgtccaccttaatgtgactagttaaggatggggcgaagaactataagcgcttgctccaacaagcgattaccgtgggtacccgtgcctttgtgtcgtgaacggtgagctatgatgatacggctacggcatcagcagccgcagcaaaagcgcaagtagcagtattaaagacagctcaggcaacaaatactcaaagcgacaaccccttcaataagtttgtccaaaaccccgattatgtccagggttcggaaaccaactgatttaacgataagagtacgccaattaaacccgattcgttactggaaagcgaaagcacttacaactttaccgcggaaccgtttgatgataagacgaaaagtcaaaagaggtctactggtggtaccactaacgaaaaacacttctttggctttaacggtttaacgattaactcgccccaaagtgtgtcgactgctagtgccggactcacggaacaaatctttaacaactttggtcagttagtaacaagtagtgacaaatccggggccttgagccagtacaaggataaagcaaccctgaaacgcttaatccaaaacactaattccgatgccgagctcaatgcctttggtgaagtgttgcaccgtgcagttaatgtcgataccagtaacttaggacgttttaactccagtggggaaccgttgattagctttgacaacaagaagaagttcttggttgatgtagtggacaaactggatgatgtttactttaacaagttcgaaggttatgttggccaaaccaaggtgaagatgtctgattcaagtagtagttcccaaggtacgaagactataagaaagccaaagccgcaccactcaccaaggactcgagtgtcaagactttgagctatgtcattcagattaccaacgaggacgttaacaaaatttctgctagttgaaaaactgataagaacagtgctttaggcctcaaaaaggaaatctttatggctttactagtagaacaagccttggacgctggtacgcaagagttagcctttttagacttagtgcgcgctaaccaggtgcaaaacaaacgtagtcaccagcaaatcctggtgggtgacaaacgcttgtatgatgcgttagggcaaacgctagcgcttagtgcgaaaaatctttaa

SEQ ID NO: 74 - protein, 1366 aa, Mycoplasma pneumoniae
MGYKLKRWPLVAFVFVAIALAVVLAASNALNTSNLFPRQNRSKQLIGFTENNIIKPEAVLKAALAEDNGTETILRVNFGEALKSWYQNNKDRNIATRLTIFSENVEDEHDNLLDQKQQAEPINWPIELQKEYDQWGGSESSWKALKLYDRLIADFQSLIFSNIVANVQLTDGSDQFKPTTKDNLDSTSNKIKFVNSKPNDPNGEFFANLQAYLFAQWVVEENPLPLTQAFFAYQAPKDGLDSLYDQAAIGSALQLGYAFPAFREPNNGQSQGKTTFDPTPNSAQNFGDFIKAVFPEQKNGQTQQSNTSSRTGLFDWQTKWNTNGAANKLLVTKSNLRGAFKGVGLATAIIDQYEYLVGGSKTSSLPEVKVDSNKSNQNPLDSFFMEGKDAVAIRSIVSRAKIAMTDQTPGFKVNPAFVKVKQSQQNDTFYQNQRKLSGGQSGDNNSQGKHHYLQDAVRLTSSQAMAAASTGADSSSGTNVGGSSGGNSVLIPLPRSAALTHTQQQVQQTTSTLQTPVYARGDDGTYALAIDGGDYFLANNKRDFTKQADILLYRYLQAKSNNFKENGVEFSLNLLESGSLFQTWAQTGLTAKLYGALVAMMGSGQGTQVKGSVQGSSRAASVSVQTTQQNRQQSTDTQESEVVKLAKSLLKSSADLAKPFTDNPTFKKALTDIQSEYKDYLAAAGKLSEFKKDLGEVSGLQQAIIDRADKYIQLEKQAQKSAIGLGQPLPYQRASDGSYPALEKFFIPEDSAADGKVKASESGSAALVTLKTTDSQKSTNTVKQPDIKPTRENNDKKLKQLTSDVETKASSLITKWGATPQIGSQFSEIVSLKSKDNKPQTNMILALLSDVGIKWTKILNSFKEWFFTNTNDFKNNYDSEKKELKGNEYKDFNDLVKQTLYLRSWQRLTSKEKFGYYKELGSVKAQAAQSGMVSLSSSAAVANAVASSGMQKSGDQTLLELGKKAFESELEASSSDGQYKYLRFLSTLMWLVKDGAKNYKRLLQQAITVGTRAFVSWTVSYDDTATASAAAAKAQVAVLKTAQATNTQSDNPFNKFVQNPDYVQGSETNWFNDKSTPIKPDSLLESESTYNFTAEPFDDKTKSQKRSTGGTTNEKHFFGFNGLTINSPQSVSTASAGLTEQIFNNFGQLVTSSDKSGALSQYKDKATLKRLIQNTNSDAELNAFGEVLHRAVNVDTSNLGRFNSSGEPLISFDNKKKFLVDVVDKLDDVYFNKFEGYVGQTKVKMSDSSSSSQGTKTIRKPKPHHSPRTRVSRLWAMSFRLPTRTLTKFLLVEKLIRTVLWASKRKSLWLYWWNKPWTLVRKSWPFWTWCALTRCKTNVVTSKSWWVTNACMMRWGKRWRLVRKIF

SEQ ID NO: 75 - DNA, 4100 bp, Mycoplasma pneumoniae
atgggttataagttaaagcgatgacctttagttgcgtttgtctttgtaggaattggcctaggggtggtgctagcggcgtctaatgcactcaatacctccaatctgtttccccgtcaaaaccgctccaagcagttgattgggtttaccgaaaacaacatcattaaacctgaagctgtactaaaagccgctttagctgaggacaatggtacggaaaccattttaagggttaactttggtgaagcgttaaagagttggtaccaaaacaataaggatcgcaacattgctacccgtttaactatctttagtgaaaacgtggaggatgaacatgataacctgttggaccaaaaacaacaggccgaaccgattaattgaccaatagagctccaaaaggaatacgaccagtggggtgggagtgaaagttcctgaaaggccttaaagttatacgaccgtttaatagccgacttccaatcacttatctttagcaacattgttgctaatgtgcagttgactgatggtagtgatcagtttaaacccactaccaaagataacctcgacagtactagcaacaaaatcaagtttgttaattccaaaccaaatgatcctaacggggagttctttgctaacctccaggcttatctatttgcccagtgagtagtggaggaaaaccccctccccctcacccaagccttcttcgcgtaccaagcacccaaggacgggctcgacagcttatacgaccaagccgcgattggtagtgccttacagttgggctatgccttccccgcgtttcgcgagcccaataatggacaaagccaaggtaaaaccaccttcgaccccacccccaacagtgcccaaaacttcggggatttcattaaggcggtgtttccggagcagaagaatggtcaaacccaacaatccaatacatcctcccgcaccgggttgtttgactggcaaaccaagtgaaacaccaatggcgctgccaataagctcttggtgaccaagtccaacctgcgcggggcctttaagggcgtggggttagctacggccattattgaccagtacgagtacctggtgggtggaagtaaaacgagttcgttgcccgaggtcaaggtagattcgaataagtccaaccaaaatccgctggatagtttttttatggaggggaaggatgcggtggcgattagatccatagtttcgcgtgcgaaaattgccatgaccgaccaaacccccgggtttaaggtcaacccggcgtttgtcaaggtgaagcagagtcagcagaacgataccttttaccaaaaccagcgcaaactgagcggcgggcaaagtggggataataactcccagggtaaacaccactacctccaagacgcggtgcggttgacgagtagtcaagcaatggcggcagcttctactggggcagattctagtagtggtaccaatgttggtggtagctccgggggcaactctgttttaattccgctcccaaggagtgccgcccttacccacacccagcaacaagttcaacaaaccacctccacactccaaacccctgtttacgcccgtggtgacgatggtacatacgccctggcaattgacggtggtgattattttttggcgaacaacaaacgcgatttcaccaaacaagccgacatcttgctgtaccgctatttacaagctaagagtaataactttaaggaaaacggcgtcgagtttagtttgaacttactggaatctggcagcctgttccaaacgtgagcacaaacggggttaaccgcgaagttgtacggtgctttggtggcgatgatgggttcgggtcaggggacccaagttaagggcagtgtgcaggggagtagccgagcagcttctgttagcgttcaaactacccagcaaaacagacagcaatcgactgatacccaagaatcggaagtggttaagttagccaagtccctgctgaaatcaagtgcggatttggccaaacccttcactgataaccccacctttaaaaaggccttaaccgacatccaatccgagtacaaggattatttggctgctgcggggaagttaagtgagtttaagaaagatttgggggaggtgagtggcttacagcaagctattattgaccgcgctgataagtacattcaactagaaaaacaagcccaaaagagtgcgattggcttgggccaaccgctcccttaccaacgcgctagtgatggtagttaccccgctttagagaagttctttattccggaagatagtgcagcagatggaaaagtaaaagcatctgaatcaggatctgccgctttagtaacactaaagacaacagatagtcaaaaaagcacaaacaccgttaaacaaccggacatcaaaccgacccgggaaaacaacgacaaaaagttaaagcagttaactagtgatgtagaaactaaagcatctagtttaatcaccaaatgaggtgcaacaccccaaattggtagtcagttctccgaaattgtttcgctcaagagtaaggacaataagccccagaccaacatgatcttggccttattgtctgatgttgggattaagtgaaccaagattctcaattcctttaaggagtggttctttactaataccaacgactttaagaacaattacgattctgaaaagaaagaacttaaaggcaatgagtacaaggactttaacgacctcgttaaacaaaccttgtacttacgttcctgacaacgactcacttccaaggaaaagtttggttattacaaggagttgggtagtgtaaaagcacaagcagctcaatcaggaatggtttcactttcaagtagcgcagcagttgcaaatgctgtagcttcaagtggcatgcaaaaatctggtgatcaaaccctcttggaactcggtaaaaaagcgtttgaaagtgaactggaagcatctagtagtgatgggcagtacaagtacctgcgcttcttgtccaccttaatgtgactagttaaggatggggcgaagaactataagcgcttgctccaacaagcgattaccgtgggtacccgtgcctttgtgtcgtgaacggtgagctatgatgatacggctacggcatcagcagccgcagcaaaagcgcaagtagcagtattaaagacagctcaggcaacaaatactcaaagcgacaaccccttcaataagtttgtccaaaaccccgattatgtccagggttcggaaaccaactgatttaacgataagagtacgccaattaaacccgattcgttactggaaagcgaaagcacttacaactttaccgcggaaccgtttgatgataagacgaaaagtcaaaagaggtctactggtggtaccactaacgaaaaacacttctttggctttaacggtttaacgattaactcgccccaaagtgtgtcgactgctagtgccggactcacggaacaaatctttaacaactttggtcagttagtaacaagtagtgacaaatccggggccttgagccagtacaaggataaagcaaccctgaaacgcttaatccaaaacactaattccgatgccgagctcaatgcctttggtgaagtgttgcaccgtgcagttaatgtcgataccagtaacttaggacgttttaactccagtggggaaccgttgattagctttgacaacaagaagaagttcttggttgatgtagtggacaaactggatgatgtttactttaacaagttcgaaggttatgttggccaaaccaaggtgaagatgtctgattcaagtagtagttcccaaggtacgaagactataagaaagccaaagccgcaccactcaccaaggactcgagtgtcaagactttgagctatgtcattcagattaccaacgaggacgttaacaaaatttctgctagttgaaaaactgataagaacagtgctttaggcctcaaaaaggaaatctttatggctttactagtagaacaagccttggacgctggtacgcaagagttagcctttttagacttagtgcgcgctaaccaggtgcaaaacaaacgtagtcaccagcaaatcctggtgggtgacaaacgcttgtatgatgcgttagggcaaacgctagcgcttagtgcgaaaaatctttaa

SEQ ID NO: 76 - protein, 1366 aa, Mycoplasma pneumoniae
MGYKLKRWPLVAFVFVGIGLGVVLAASNALNTSNLFPRQNRSKQLIGFTENNIIKPEAVLKAALAEDNGTETILRVNFGEALKSWYQNNKDRNIATRLTIFSENVEDEHDNLLDQKQQAEPINWPIELQKEYDQWGGSESSWKALKLYDRLIADFQSLIFSNIVANVQLTDGSDQFKPTTKDNLDSTSNKIKFVNSKPNDPNGEFFANLQAYLFAQWVVEENPLPLTQAFFAYQAPKDGLDSLYDQAAIGSALQLGYAFPAFREPNNGQSQGKTTFDPTPNSAQNFGDFIKAVFPEQKNGQTQQSNTSSRTGLFDWQTKWNTNGAANKLLVTKSNLRGAFKGVGLATAIIDQYEYLVGGSKTSSLPEVKVDSNKSNQNPLDSFFMEGKDAVAIRSIVSRAKIAMTDQTPGFKVNPAFVKVKQSQQNDTFYQNQRKLSGGQSGDNNSQGKHHYLQDAVRLTSSQAMAAASTGADSSSGTNVGGSSGGNSVLIPLPRSAALTHTQQQVQQTTSTLQTPVYARGDDGTYALAIDGGDYFLANNKRDFTKQADILLYRYLQAKSNNFKENGVEFSLNLLESGSLFQTWAQTGLTAKLYGALVAMMGSGQGTQVKGSVQGSSRAASVSVQTTQQNRQQSTDTQESEVVKLAKSLLKSSADLAKPFTDNPTFKKALTDIQSEYKDYLAAAGKLSEFKKDLGEVSGLQQAIIDRADKYIQLEKQAQKSAIGLGQPLPYQRASDGSYPALEKFFIPEDSAADGKVKASESGSAALVTLKTTDSQKSTNTVKQPDIKPTRENNDKKLKQLTSDVETKASSLITKWGATPQIGSQFSEIVSLKSKDNKPQTNMILALLSDVGIKWTKILNSFKEWFFTNTNDFKNNYDSEKKELKGNEYKDFNDLVKQTLYLRSWQRLTSKEKFGYYKELGSVKAQAAQSGMVSLSSSAAVANAVASSGMQKSGDQTLLELGKKAFESELEASSSDGQYKYLRFLSTLMWLVKDGAKNYKRLLQQAITVGTRAFVSWTVSYDDTATASAAAAKAQVAVLKTAQATNTQSDNPFNKFVQNPDYVQGSETNWFNDKSTPIKPDSLLESESTYNFTAEPFDDKTKSQKRSTGGTTNEKHFFGFNGLTINSPQSVSTASAGLTEQIFNNFGQLVTSSDKSGALSQYKDKATLKRLIQNTNSDAELNAFGEVLHRAVNVDTSNLGRFNSSGEPLISFDNKKKFLVDVVDKLDDVYFNKFEGYVGQTKVKMSDSSSSSQGTKTIRKPKPHHSPRTRVSRLWAMSFRLPTRTLTKFLLVEKLIRTVLWASKRKSLWLYWWNKPWTLVRKSWPFWTWCALTRCKTNVVTSKSWWVTNACMMRWGKRWRLVRKIF

SEQ ID NO: 77 - DNA, 1017 bp, Mycoplasma penetrans
atgataaataatagaatttgtatattagggaccggagcttgagcaactgcacttggttctagattatctttaaatggtaatactgtttttctttgaggaattgataacaatgaagtaaatgatattaatagtggatacaataaaaaatattttggaaacactaaatttagttcatcattatcagctactacagatttaaaaacagcaattggggattctaaatatattatttttgcaattccttcaactgcactagattcagtattagataaagtaaaagaatttttgtctgataaaaaatctcaagttattttaattaatgttgttaaaggaatagatgcagaaactagtcaaatcctaagtaacaaaattaaatctaaattaggaagtcattattattctagacttgtaactttatgtggtccatcatttgctactgaagtttttgaagagaaacctactgttatcaatggaactggaaaaaaccttaaaattgtaaaacaagtatgtgagttatttaacagtgatgttttcaaagttattccaattaatgatattgttggtttacaagtattttctagtttaaaaaatttattagcaattgcagttggattatcacaagcagaatatacttctgtaaatacaatgtcagcaatcttaacaatggggatagaagagattcaaaaaatagcttgtaaaatgaaagcaaaaaaatgaacaattatgtcattttgcgggattggtgatatcttcttaacatgtagttctactcaatctagaaattatagttttggacaagacttattaaaaaaaggtgttgaaaaaactattaaggaaaataaaaagacagttgaaggatttgaagtttataaaactgctaaaaatattattactaaatataatatcaatgcaccaatctttagttctattatagaagtattagaaggaaaactagaccctaaagaattttctaaaaagtgtttagaattattctgaaaccaaaaaattaataaataa

SEQ ID NO: 78 - protein, 338 aa, Mycoplasma penetrans
MINNRICILGTGAWATALGSRLSLNGNTVFLWGIDNNEVNDINSGYNKKYFGNTKFSSSLSATTDLKTAIGDSKYIIFAIPSTALDSVLDKVKEFLSDKKSQVILINVVKGIDAETSQILSNKIKSKLGSHYYSRLVTLCGPSFATEVFEEKPTVINGTGKNLKIVKQVCELFNSDVFKVIPINDIVGLQVFSSLKNLLAIAVGLSQAEYTSVNTMSAILTMGIEEIQKIACKMKAKKWTIMSFCGIGDIFLTCSSTQSRNYSFGQDLLKKGVEKTIKENKKTVEGFEVYKTAKNIITKYNINAPIFSSIIEVLEGKLDPKEFSKKCLELFWNQKINK

SEQ ID NO: 79 - DNA, 906 bp, Mycoplasma pneumoniae
atgaaagggttttcttgctccagaccgggctatcttactggtctcttgctcctcgctgtagcccctattctcactgcttgtacccgcgactatactaccaagaacgaatttcaattaacaacagctcaacaggccaaattaaagccagccaccatcgaatattggcgtgatggtgatacgcctgagatcaattacgcaagcgaagagcgccgaaaggaagctgaacaaaaatctaaagaaaacgctaagaaagaagacaaaaaagaggaaaagaaaactgaagattcccaagattcttcttctgcatccacgcaagttcgtagtagtaaacacggtctccgcatctatggaatagatacacccgaaaaacatgtaagtagcaaaggcgatagcaccggtgatgaaaaaatcgaagccgaaaaggcctctaactatgctgaaaagctaatccctaaaggcagtactgtttgggtttgatcactcaacacttacagctatgatcgtgaagtgggcgctttattctttaagtctaaccctaagcaaaccttcttccaatcctttgaagtagctatggttgaggctggtcatgctattccaattgctgggactggtttaaatttaatcgctgatccggaattaagtgctgatgatcctttaagtgtcattggtctacagttagccaacgctgccaataaggcttacaacgctaaaattaacatttggagccatgacactgatggttatcgttccttgactgctgtttacaagttaaggggcgctgatattagttgaacccgtttcctagatgaagccaatggatattcctctgctagtgctggaaccggtgctagtttgtaccaactatgagatcaacgtcaagccaaattagcccaaaagggtagttaa

SEQ ID NO: 80 - protein, 301 aa, Mycoplasma pneumoniae
MKGFSCSRPGYLTGLLLLAVAPILTACTRDYTTKNEFQLTTAQQAKLKPATIEYWRDGDTPEINYASEERRKEAEQKSKENAKKEDKKEEKKTEDSQDSSSASTQVRSSKHGLRIYGIDTPEKHVSSKGDSTGDEKIEAEKASNYAEKLIPKGSTVWVWSLNTYSYDREVGALFFKSNPKQTFFQSFEVAMVEAGHAIPIAGTGLNLIADPELSADDPLSVIGLQLANAANKAYNAKINIWSHDTDGYRSLTAVYKLRGADISWTRFLDEANGYSSASAGTGASLYQLWDQRQAKLAQKGS

SEQ ID NO: 81 - DNA, 1776 bp, Mycoplasma pneumoniae
atgccaaatcctgttagatttgtttaccgtgttgatttgagaagccctgaagaaatttttgaacatggcttttcaactttaggtgatgtgagaaatttctttgaacacattctctccactaattttggtagaagctattttatttccacttcagaaacacccacagcagctattcgcttctttggtagctggttacgggaatatgtaccagagcaccccagaagggcttacttatatgaaattcgtgccgaccaacacttttacaatgcccgcgccactggggagaacttgttagatttaatgcgtcaaagacaagtagtatttgactctggtgatcgagaaatggcacaaatgggaattagagctttacgcacttcctttgcgtatcaacgtgaatggtttaccgatggtccaattgcagcagctaatgtccgtagtgcttgactagtagatgctgttcccgttgaacctggtcatgctcaccacccggctggtcgtgttgtagagactactagaattaatgaaccggaaatgcacaaccctcattatcaagagctgcaaacccaagccaatgatcaaccatgattgccaacaccaggaatagctactcctgtacatttatcaattccccaagcagcttccgttgctgatgtttcggaaggtacttccgcttcgctatcgtttgcgtgccctgattgaagtccaccttctagtaatggtgaaaatccgctagacaaatgcattgcggaaaagattgataactataacctacaatccttaccacagtacgctagcagtgtaaaggaactggaagatacaccagtatacctaaggggaattaaaacgcaaaaaacctttatgttacaagcagatccgcaaaataacaatgtctttttggtcgaagtaaaccccaaacaaaagtccagctttccccaaaccatcttcttttgggatgtttatcaacgaatttgtctcaaggatttaactggtgcacaaatcagtctttcgcttactgcctttactactcagtatgctggtcagctcaaagtgcaccttagtgttagcgcggttaatgccgtgaaccaaaagtgaaaaatgacaccgcaagacattgcaataactcagtttcgggtctcctctgaactgttaggtcaaactgaaaatggcttgttctgaaataccaagagtggtggttcacaacacgatttgtatgtatgtcctttgaaaaatccacctagtgatttggaagaattacaaataattgttgatgaatgtactacccatgcgcagtttgttactatgcgtgcagctagcaccttctttgttgatgttcagctaggctggtattgaaggggttattactataccccacaattaagtggttgatcttatcagatgaaaacaccagatggacagatattctatgatctaaaaacttcgaaaatcttctttgtccaggacaaccaaaacgtgttctttctccataataaactcaacaaacaaactggttacagctgggattgagtagaatggctaaaacatgacatgaatgaggacaaagacgaaaactttaaatggtacttttcgcgtgatgaccttaccattccttccgttgaagggcttaacttccgccacattcgctgttacgctgacaaccagcagttaaaggtgatcataagcggttcacgttggggcggttggtactccacttacgataaagttgaaagtaatgtcgaagataagattttggtcaaagatggttttgatcgcttttag

SEQ ID NO: 82 - protein, 590 aa, Mycoplasma pneumoniae
MPNPVRFVYRVDLRSPEEIFEHGFSTLGDVRNFFEHILSTNFGRSYFISTSETPTAAIRFFGSWLREYVPEHPRRAYLYEIRADQHFYNARATGENLLDLMRQRQVVFDSGDREMAQMGIRALRTSFAYQREWFTDGPIAAANVRSAWLVDAVPVEPGHAHHPAGRVVETTRINEPEMHNPHYQELQTQANDQPWLPTPGIATPVHLSIPQAASVADVSEGTSASLSFACPDSPPSSNGENPLDKCIAEKIDNYNLQSLPQYASSVKELEDTPVYLRGIKTQKTFMLQADPQNNNVFLVEVNPKQKSSFPQTIFFWDVYQRICLKDLTGAQISLSLTAFTTQYAGQLKVHLSVSAVNAVNQKWKMTPQDIAITQFRVSSELLGQTENGLFWNTKSGGSQHDLYVCPLKNPPSDLEELQIIVDECTTHAQFVTMRAASTFFVDVQLGWYWRGYYYTPQLSGWSYQMKTPDGQIFYDLKTSKIFFVQDNQNVFFLHNKLNKQTGYSWDWVEWLKHDMNEDKDENFKWYFSRDDLTIPSVEGLNFRHIRCYADNQQLKVIISGSRWGGWYSTYDKVESNVEDKILVKDGFDRF

SEQ ID NO: 83 - DNA, 114 bp, Artificial Sequence
  Deletion template MPN257
  misc_feature (1)..(1): N(1) is cytosine wherein a phosphorothioate bond substitutes a sulfur atom for a non-bridging oxygen in the phosphate backbone
  misc_feature (2)..(2): N(2) is guanine wherein a phosphorothioate bond substitutes a sulfur atom for a non-bridging oxygen in the phosphate backbone
  misc_feature (3)..(3): N(3) is adenine wherein a phosphorothioate bond substitutes a sulfur atom for a non-bridging oxygen in the phosphate backbone
  misc_feature (4)..(4): N(4) is thymine wherein a phosphorothioate bond substitutes a sulfur atom for a non-bridging oxygen in the phosphate backbone
nnnnaaaaaattgtttcatcgctcagcattttgtgtaatgtaccgttcgtataatgtatgctatacgaagttataacgagtactttactcttggtttcactcatcaaaaccttc

SEQ ID NO: 84 - DNA, 1017 bp, Mycoplasma pneumoniae
atgagtgaaaccaagagtaaagtactcgttttaggcggtttaggttacattgggagctgttttattgaccagcttctaaaacagtaccctgatgtaactgttagtgtaattgacattaaccacactagtctggcgctacaactgttaccaagacaggtcaacgttcactttgttaacttacttgaccgtgcacagttaacagatacgattgctcaaattaaccctgatgtcgtgtttcactttgcggcaaaaacaagcgtaaaggaatcgaccgaacaaccacttacatattttgaccataacctagtcggtacgctcaacttactccacgctttaaaagaactacaaaaaccgatccagctgttcttctcctccactgctgcagtctttggtagtgctagtacacttccaattccagaaaacttagttttagaggaaacactcgcttccaatccgtatggcattagtaagtttttaagtgaaatagtactgcaaacacttacccgtagcccccattttcaagtaatagcgttgcgctactttaacgtagcgggtgctagtaatccctttggtaacttcaataaaaacacgaccttactgattcctaacctgattaaagcctttatggaaaagcgcaccttctttctgtatggtgatgactatgacactaaagatgggagctgtatccgtgattacatccatgtagtagacttgtgtgatgcccatttgttggcgtgaaagtggttgcaagccaatcctaaagtgcgctttgaaagctttaacttaggtagtggtcagggcttttccaattgggaggtcattaatactgctcaagcaatctttgcgcctgaacaattacagctcaaaattgaatcacgaagggctggtgatccacctgttttagtggttgattgcacgaaggcaaaacgcttactcaacttccaaccaacccgctcattacacaaaatgctgagcgatgaaacaattttttatcgggatttttataataggctttaa

SEQ ID NO: 85 - protein, 338 aa, Mycoplasma pneumoniae
MSETKSKVLVLGGLGYIGSCFIDQLLKQYPDVTVSVIDINHTSLALQLLPRQVNVHFVNLLDRAQLTDTIAQINPDVVFHFAAKTSVKESTEQPLTYFDHNLVGTLNLLHALKELQKPIQLFFSSTAAVFGSASTLPIPENLVLEETLASNPYGISKFLSEIVLQTLTRSPHFQVIALRYFNVAGASNPFGNFNKNTTLLIPNLIKAFMEKRTFFLYGDDYDTKDGSCIRDYIHVVDLCDAHLLAWKWLQANPKVRFESFNLGSGQGFSNWEVINTAQAIFAPEQLQLKIESRRAGDPPVLVVDCTKAKRLLNFQPTRSLHKMLSDETIFYRDFYNRL

SEQ ID NO: 86 - DNA, 114 bp, Artificial Sequence
  Deletion template MPN483
aatgaacaagctaattagcattctggttccttgttaccaataccgttcgtataatgtatgctatacgaagttatctaacaagctaaagcttcgagtagctttttaaaaagttgt

SEQ ID NO: 87 - DNA, 1026 bp, Mycoplasma pneumoniae
atgaacaagctaattagcattctggttccttgttaccaatcccagccctttctcgatcgctttttcaaatcacttttaaagcaagactggaacggtgttaaagtaatcttctttaatgataacaagccagatccaacatacgagatcctaaaacaatttcaacaagcacatccacaactagcgattgaagtacattgtggtgagaaaaatgtcggtgtaggcggttcacgtgatcaactaataaattatgtagacacaccctacttttactttgttgatcctgatgatgaatttagtgatcccaactgttttaaggcaatagtggaaacaattcaaggagaaaactttgatattgcagtgttaaacagtattgtttacctccaaatgttgaagaatgactttttaattaaacacattccgctcaaaaacatctttcagggcaaggtaaaactcaatcctgataacaccgtaaatcatttgcactatatccagaataacgaccagtacatttggaacattgtcatcaacaccgcctttttcaaagcattggatttgcagtttgttaaccgctttattgaagacattgcggtgtggtttccgattatgtttaaagcccaaaaagtgctgtgaattgatgtaaatggggttaactattacttgcgaccgaacagtgcttccacccagaaaaacagcattaaattactctcctttattgaggcttacgaacggttgtatttccacttaaagaaggtgggtaagctagctgattttattgaccctaacaataagattgaaagtcgtttttgacggcgtcaagccttcatttggtttagttttattaatgtttcatggatgaaagcggaatttgaacagaccaaaagtgtgttgcaaaaactgtttgacttcatggaagccaacggcatctacgacagggtatttactaataagcaccacggtatttacttattgtgagttaaccgcttaaaacactttaagaagttagtgcaagcacaaccacatctataa

SEQ ID NO: 88 - protein, 341 aa, Mycoplasma pneumoniae
MNKLISILVPCYQSQPFLDRFFKSLLKQDWNGVKVIFFNDNKPDPTYEILKQFQQAHPQLAIEVHCGEKNVGVGGSRDQLINYVDTPYFYFVDPDDEFSDPNCFKAIVETIQGENFDIAVLNSIVYLQMLKNDFLIKHIPLKNIFQGKVKLNPDNTVNHLHYIQNNDQYIWNIVINTAFFKALDLQFVNRFIEDIAVWFPIMFKAQKVLWIDVNGVNYYLRPNSASTQKNSIKLLSFIEAYERLYFHLKKVGKLADFIDPNNKIESRFWRRQAFIWFSFINVSWMKAEFEQTKSVLQKLFDFMEANGIYDRVFTNKHHGIYLLWVNRLKHFKKLVQAQPHL

SEQ ID NO: 89 - DNA, 1026 bp, Mycoplasma genitalium
atggataaacttgttagtatattagttccttgttacaaatcaaaaccttttttaaaacgtttttttaattcacttttaaagcaagatcttaatcaagctaaaattatttttttcaatgacaatgttgctgatgaaacctatgaagttttgcaaaaattcaaaaaagaacacaataacttagcaattgaagtctattgtgacaaacagaatgaaggtattggaaaagtgcgtgataagctagtgaatctagtaacaacaccttatttttattttatcgatcctgatgattgttttaacaacaaaaatgtcataaaagagattgttgaatcaattaaaaaagaagattttgatcttggtgtattaaaaagtatggtctatttatgcttcttaaaacatgatttcattattaaatttttgcctttaaaaggtatttttcaaggcagagtaaaattaattaataataacaatgttaataaattaaattacatcaaaaataatgatcaatatatttgaaatattgttataaacacagatttttttaggaagctaaatttaacttttgaatcaaggttatttgaagatataccaatctgatatccgatgtttttttcatcacaaaaaattgtttttattgatgtgataggaacaaattattttattcgtaatgatagtttatcaactactattagtgctccacgctacttaaatttaatccaatgttatgaaaagctatatgtaaatctcagccaaaatggttctcttgcaagttttattgatccaaatcataagattgaagctaggttttgaagaaggcaaatgtttgtttgatttgcacttttcagctttgaatactttaagaaaaatttttctgaatcaaaaaaaattcttgaaaaactatttgtttttttggaaaaaaatggagtttatgaacgtgtttttcaaacaaaaaatcaaggtatttactatatttgggtacagcgactaaaatattttaaacatgttttggaatctaaatcagataactaa

SEQ ID NO: 90 - protein, 341 aa, Mycoplasma genitalium
MDKLVSILVPCYKSKPFLKRFFNSLLKQDLNQAKIIFFNDNVADETYEVLQKFKKEHNNLAIEVYCDKQNEGIGKVRDKLVNLVTTPYFYFIDPDDCFNNKNVIKEIVESIKKEDFDLGVLKSMVYLCFLKHDFIIKFLPLKGIFQGRVKLINNNNVNKLNYIKNNDQYIWNIVINTDFFRKLNLTFESRLFEDIPIWYPMFFSSQKIVFIDVIGTNYFIRNDSLSTTISAPRYLNLIQCYEKLYVNLSQNGSLASFIDPNHKIEARFWRRQMFVWFALFSFEYFKKNFSESKKILEKLFVFLEKNGVYERVFQTKNQGIYYIWVQRLKYFKHVLESKSDN

SEQ ID NO: 91 - DNA, 1032 bp, Mycoplasma agalactiae
atgaataagagcaataagcataaatcgagaaaaaacaataacgctaatcctaaaattagcgtattaattccttgccataatatagaaaacaactcttatttttccttagaaagtgttttaaaaagctcgtataaaaatttagaaattgtattgttaaatgactactcaacagataacacactgcaaataattaacgaatatgctgttaaagattcaagaattaaagtctatgatttaaaagattaccacgaacatgtaggtataggttttaacagacactttctgattgagaaggcgactggcaaatattttatttttgttgatgacgatgacaaattacacagaaatgctataaaaacttttgtggacaatttggatgatgactatgacctattagttgctgaagctttatataattatgaatatgcaaaaaacttaagaatttcggtgcctgaggtgcctcactttcttaattatgatattaactctcccatcgagcactactataataatgtttcagtttgttgaggaaagctaattaaaagggatttttactttcaaattgttaaaaaatataataaaaagttttctgatggaatatatgaagatattcgctttatgcatatgcttttctttacaaatgttaagtttaaaagaataaaaaagaaagtttacacataccaaattcgtaaaaattcgtcagctactaatttaagcgcttgatctgataagctcaaattacttttttcctcataccaaagcgtttttgatgatttttctaacctaaagttagttgaagatgacaagttattgaaatcattaaaaaagggcttgtttgtgcagttgttatcgctttgttacttgctttgcatgattcaaaaacgcgaatgtaggacaaaaatgcagcaaatttgttctttaatgcttaaagagtttataaaaaacaatgatattaatattgctgtgccttcaaatttatcattatggtcactaatgatttataagttgtcaataaagcactttaagttgtaa

SEQ ID NO: 92 - protein, 343 aa, Mycoplasma agalactiae
MNKSNKHKSRKNNNANPKISVLIPCHNIENNSYFSLESVLKSSYKNLEIVLLNDYSTDNTLQIINEYAVKDSRIKVYDLKDYHEHVGIGFNRHFLIEKATGKYFIFVDDDDKLHRNAIKTFVDNLDDDYDLLVAEALYNYEYAKNLRISVPEVPHFLNYDINSPIEHYYNNVSVCWGKLIKRDFYFQIVKKYNKKFSDGIYEDIRFMHMLFFTNVKFKRIKKKVYTYQIRKNSSATNLSAWSDKLKLLFSSYQSVFDDFSNLKLVEDDKLLKSLKKGLFVQLLSLCYLLCMIQKRECRTKMQQICSLMLKEFIKNNDINIAVPSNLSLWSLMIYKLSIKHFKL

SEQ ID NO: 93 - DNA, 1149 bp, Bacillus subtilis
ttgaataccaataaaagagtattaattttgactgcaaattacggaaatggacatgtgcaggtagccaaaacactttatgaacaatgtgtacggctcggctttcagcatgtaacagtttctaatttgtaccaagagtcaaatccgattgtttcagaggtaactcaatacctttatttaaaaagcttctcaatcgggaaacagttttatcgtttgttttattacggagttgacaaaatctataataaacgtaaattcaatatttactttaaaatgggtaataaaagattgggcgaacttgtcgatgaacatcagcccgatattattattaatacatttccgatgatcgtcgtgccggaatacagacgccgaactggaagagtcattcctaccttcaacgttatgactgatttttgtcttcataaaatttgggttcacgaaaacgtggataaatattatgtggcgacagattacgtgaaggaaaaactgctggagatcggcactcatccaagcaatgtaaaaatcacaggaattccaatcaggccgcaatttgaagaatccatgcctgttggcccgatatataaaaagtacaatctttcaccaaacaaaaaagtgcttctgatcatggcaggtgctcacggtgtattaaagaacgtaaaagagctgtgcgaaaaccttgtcaaggatgaccaagtgcaagtagttgtcgtgtgcgggaaaaatacggctttaaaagaatctttgagtgcgcttgaagcggaaaatggtgacaaattaaaagttctgggctatgtggagcgcattgatgagctatttcggatcacagattgcatgattaccaagcccggcggcattactttgacagaagccacagccattggagtgcctgtcattctgtacaaacccgtgcctggccaggaaaaagaaaatgcaaacttctttgaagaccgcggagctgccatcgttgtgaaccgtcatgaagagattctcgagtcagtcacttcccttcttgcagatgaagataccttgcatcgcatgaagaaaaacattaaggaccttcatttagcaaactcctctgaagtgattttagaggatatcctgaaggaatcagaaatgatgaccgccaaacaaaaagccaaagtgctatcgtaa

SEQ ID NO: 94 - protein, 382 aa, Bacillus subtilis
LNTNKRVLILTANYGNGHVQVAKTLYEQCVRLGFQHVTVSNLYQESNPIVSEVTQYLYLKSFSIGKQFYRLFYYGVDKIYNKRKFNIYFKMGNKRLGELVDEHQPDIIINTFPMIVVPEYRRRTGRVIPTFNVMTDFCLHKIWVHENVDKYYVATDYVKEKLLEIGTHPSNVKITGIPIRPQFEESMPVGPIYKKYNLSPNKKVLLIMAGAHGVLKNVKELCENLVKDDQVQVVVVCGKNTALKESLSALEAENGDKLKVLGYVERIDELFRITDCMITKPGGITLTEATAIGVPVILYKPVPGQEKENANFFEDRGAAIVVNRHEEILESVTSLLADEDTLHRMKKNIKDLHLANSSEVILEDILKESEMMTAKQKAKVLS

SEQ ID NO: 95 - DNA, 426 bp, Mycoplasma pneumoniae
atggacaaaaagtacgacatcactgctgttttaaacgaagacagctccatgactgccatcagtgatcagtttcaaattacactcgatgcccgtcccaagcacaccgctaagggttttggtcctttagctgctctgctctctggtttagctgcttgtgaattggcaaccgctaacttgatggcaccagctaaaatgattaccatcaataaactgttgatgaatgtcactggttcacgctcaaccaaccccaccgatggttattttggtttaagggaaattaacctgcactgggaaatccactcccctaacagtgaaacggaaatcaaggagttcattgactttgtttccaaacgctgcccagcgcacaatacgctccaaggtgtgagtcaactcaagattaacgttaatgtaactttagtgcattaa

SEQ ID NO: 96 - DNA, 134 bp, Mycoplasma pneumoniae
ttgggtaaaaaacgagcgaaaaatgctgaaaaaatacctgagtctttcaggtatttttttatgcaaaatttgccctaaaaaaagagtaaatttgagaaattgatccgttgttcattccgggataattttcccgt

SEQ ID NO: 97 - DNA, 96 bp, Mycoplasma pneumoniae
atgaaactacaaatttttaatacaaagctaggattactagcagctaagatttactgaaagtcctggagacacctaggactgtctgaagatgaaatt

SEQ ID NO: 98 - DNA, 34 bp, Artificial Sequence
  Lox LE2272
taccgttcgtataaagtatcctatacgaagttat

SEQ ID NO: 99 - DNA, 22 bp, Mycoplasma genitalium
tagtatttagaattaataaagt

SEQ ID NO: 100 - DNA, 1920 bp, Streptococcus suis
atgaaaattattaatattggagttttagctcatgttgatgcaggaaaaactaccttaacagaaagcttattatataacagtggagcgattacagaattaggaagcgtggacaaaggtacaacgaggacggataatacgcttttagaacgtcagagaggaattacaattcagacaggaataacctcttttcagtgggaaaatacgaaggtgaacatcatagacacgccaggacatatggatttcttagcagaagtatatcgttcattatcagttttagatggggcaattctactgatttctgcaaaagatggcgtacaagcacaaactcgtatattatttcatgcacttaggaaaatggggattcccacaatcttttttatcaataagattgaccaaaatggaattgatttatcaacggtttatcaggatattaaagagaaactttctgccgaaattgtaatcaaacagaaggtagaactgtatcctaatgtgtgtgtgacgaactttaccgaatctgaacaatgggatacggtaatagagggaaacgatgaccttttagagaaatatatgtccggtaaatcattagaagcattggaactcgaacaagaggaaagcataagatttcagaattgttctctgttccctctttatcatggaagtgcaaaaagtaatatagggattgataaccttatagaagttattactaataaattttattcatcaacacatcgaggtccgtctgaactttgcggaaatgttttcaaaattgaatatacaaaaaaaagacaacgtcttgcatatatacgcctttatagtggagtactacatttacgagattcggttagagtatcagaaaaagaaaaaataaaagttacagaaatgtatacttcaataaatggtgaattatgtaagattgatagagcttattctggagaaattgttattttgcaaaatgagtttttgaagttaaatagtgttcttggagatacaaaactattgccacagagaaaaaagattgaaaatccgcaccctctactacaaacaactgttgaaccgagtaaacctgaacagagagaaatgttgcttgatgcccttttggaaatctcagatagtgatccgcttctacgatattacgtggattctacgacacatgaaattatactttctttcttagggaaagtacaaatggaagtgattagtgcactgttgcaagaaaagtatcatgtggagatagaactaaaagagcctacagtcatttatatggagagaccgttaaaaaatgcagaatataccattcacatcgaagtgccgccaaatcctttctgggcttccattggtttatctgtatcaccgcttccgttgggaagtggaatgcagtatgagagctcggtttctcttggatacttaaatcaatcatttcaaaatgcagttatggaagggatacgctatggttgtgaacaaggattgtatggttggaatgtgacggactgtaaaatctgttttaagtatggcttatactatagccctgttagtaccccagcagattttcggatgcttgctcctattgtattggaacaagtcttaaaaaaagctggaacagaattgttagagccatatcttagttttaaaatttatgcgccacaggaatatctttcacgagcatacaacgatgctcctaaatattgtgcgaacatcgtagacactcaattgaaaaataatgaggtcattcttagtggagaaatccctgctcggtgtattcaagaatatcgtagtgatttaactttctttacaaatggacgtagtgtttgtttaacagagttaaaagggtaccatgttactaccggtgaacctgtttgccagccccgtcgtccaaatagtcggatagataaagtacgatatatgttcaataaaataacttag

SEQ ID NO: 101 - DNA, 34 bp, Artificial Sequence
  Lox R5171
ataacttcgtataatgtgtactatacgaacggta

SEQ ID NO: 102 - DNA, 1719 bp, Mycoplasma pneumoniae
ctagatgaagatatccttcttcttgaagaatttttccactagcttttccacctccttggcgttcgctaaggttaatgctttttgtgctaatgtctgacactctgtgtaattgagcttagcgataatcatccgcgccttaaacatggaactagcactcatggaaaactctttgagacccaaacctaaaagtaggggaatcgctaacggttcaccggccatctcaccacacattcccgttcaaatatcattcactttacccccttcaatggtgaggtaaatgagttttaataaagctgggtttaaaggttggtagagataactaacattcttattcatccggtcagccgcaaagctgtactggatcaggtcattggtaccaatggagaagaaatccacgtggtgacttaaggtgttagccgctaaaacagcagcggggatttcgatcataatcccgagcttaaactgtttattgccacccaactcctggtgcacctggtttaacagttgcttggcttgcaacagttcatccaaggtagcgaccatcgggaacataatgcccaactgaccgtgatcagcagcacgtagtaaagcccgcaattgtgttttaaagatgtcctgtttgtctaaggttaaacgcaaagcgcggtagcccaagaaggggttatcttcgtgaggaaactcatagtaattaagcttcttgtccccaccaatgtctagggtgcggataataactaaatcaccgtgcgcttgttctaacactgctttgtaagcctggtactgcgtttcttcatcgggtcattcttgactgttcatgtagaggaactcagtgcggaataaaccaaccccgttggtattgtactgacatgccaattccatgtctttcacattaccaatgttagcagccaccacaaccgcatgaccatctaaagtcacggtttccttgttggtgtactgcttgagttcttgttggtatttttcttccaacgctttttgtttcttccactcggtagtgtccttggagtttaggtctaagccagctattcctttgcgcccatcaaccccaactatttgaccgtctttgaccttagtagtaatgcccttaatgcccaccactgcgggaatttccagtgaacgcgccataatggcagcgtgactggtacgaccacctatttcggttaaaaaacccttgacgtattttttgttaagggtagcagtttgactcggcgttaagtcatgggccacaataatgacatcactcttaattttgaccaggtcatgtggtttttgtccggttaaataagctagtaagcgttggcgcaagtccaaaatgtctgcagcacgttcctggaaatacttgtcctgcatttggctaaacatggtagccgtttggctaaagacagtgtccacggctataacaggatgaacattttgcgctaactgttgttctagttgctcggtgatcgtgggatcgttgagcatttgaatgtgcgcatcaaagatcatgcccgcttcagtgttaatgttgttaacggtaatttccttaatttcctctaagtccttaacggcttttttgaaagctgagtccaaaagtcttttggcctgcgccttcgttaattggtgcttgaggtatttgttaacagcgaattccggtgttttaacaaggaaggctttggctagggccatgccatcagatacaccgataccagatagttttttcat

SEQ ID NO: 103 - DNA, 771 bp, Mycoplasma pneumoniae
ttgggcctccagttaagtggcctggatgccagtgacagcgaccaaagggagttaatttgggccaagcggccctgagcggcatttcgtggcagttgggtcaaccggttgggacgggtcgagagtgtgtgggatttgaagggggtgtgggcggatcaagctcacagcgcggtgtccgagtcgcaagcagctacaagtagtaccaccaccaccgcaacaggggacaccttaccggagcacccgaatgccctcgcgtaccaaattagctccaccgacaaggattcgtacaaggcttccactcaaggctccggtcaaaccaattcccaaaacacctccccctacctgcatttgattaaacctaaaaaagttactgcttccgacaagttagacgacgatcttaaaaacctgttggaccccaacgaggttcgggtgaagctgcgccaaagctttggtacagaccattccacccaaccccaaccccaacccctcaaaacaacgacaccggtgtttgggacgaatagtggtaacctcggtagtgtgcttagtggtgggggtaccacgcaggactcaagcaccaccaatcaactgtcacccgttcaacgggtgagtgggtggcttgtggggcagttaccaagcacgagtgacggaaacacctcctccaccaacaacctcgcgcctaatactaatacggggaatgaggtggtgggggtgggagatttgtctaagcgagcttccattgaatcaagtcgtttgtgaatagcattaaaaccttag

SEQ ID NO: 104 - DNA, 457 bp, Mycoplasma pneumoniae
ctaacgctaactttgggcgatttaatccacttagttttgaactccttaaatttgtgtttttcttgactttgctaaaaaaacaattcaaagaggcttgcaatgaacaggtcagcactgctaaattggtgagtgcaagaactaaataaaagtaattattttgtaatttttcggcggtcttgccatacatcaattaaatgcttgatcaaaaccaaggtgtcaaaatttagtgcacaattgctaagagcgcctgtatatttagagtaaaactacttaaattgctaaaaattaaccgtttttgaattagttttagggtcaaaattaggccaatttcccgctttctttttttaaaaaatctctactttttaccccctaaataacctaacaactacacaattaattcccaattaatgtaaatttatctaaattgttcaccgtagttagttcaatttaggtaaag

SEQ ID NO: 105 - DNA, 34 bp, Artificial Sequence
  Lox71
taccgttcgtataatgtatgctatacgaagttat

SEQ ID NO: 106 - DNA, 22 bp, Mycoplasma pneumoniae
tagtatttagaattaataaagt

SEQ ID NO: 107 - DNA, 661 bp, Muromegalovirus
atggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaattccgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtctgtgatggcttccatgtcggcagaatgcttaatgaattacaacagtactgcgatgagtggcagggcggggcgtaac

SEQ ID NO: 108 - DNA, 34 bp, Artificial Sequence
  LoxM2/66
ataacttcgtatatggtttcttatacgaacggta

SEQ ID NO: 109 - DNA, 23 bp, Mycoplasma pneumoniae
tgaaagcagtttaaaccagcaat

SEQ ID NO: 110 - DNA, 1761 bp, Mycoplasma pneumoniae
atggaaaagatacaagcggaaaaatcccaaagtgcaattgagtttaagaatattgtagtcgactttggtgagtcaatcgcaattgataacattaacctcacggttaaaaagaaggagttagttaccttactaggtccttctgggtgtgggaaaacaacttcgctttccgtgattgcgggtcttatcgcccctactagtggtcaggttttatttaatggttatgatgtcacaaaaaagccaccacaacaacgcaaactgggattagtgttccaaaactatgccttgtatccgcacatgagtgtgtttgaaaacattgtcttcccactctacagtgatacgtcttgacgcgaagccatctttgaaaaaaatacctgagcgcaacacgacattaattgcttgatcttaaaggctaatggtgctactagtgaggagctggcagagctcaatcgtttaatgcaacaacgaatcgatgagcccaaacgaatggcttaccagattaatgatttgatggtaagtgtgtttcaaaaacagagtgaactggaagctaacctaaagttaattccccgcaaaaaacagtttgccattatttcactcagcaaggaaacactaagtcaaataagggatgttgaaactaaagccaaggctgcattggaaaccgctgatagtgctgaagtggagcagaccattaaaagtgaacttaagcaaaaactaagtgaaattaaggctaattaccacgatgaaaaggccaacattaaagcttactgatgggaaatgttggccaatattaagaccgaattaaaaaccgaaaaaactgccattaagcaaactaatgactatgccaaattgaaggaattgaagtgaaagatccactttgaacccttgaatttaaagaagcagtaccgcagttactttaaacaactaaaagccaaatacagcttaaaagatggcaatttaactgaaagcgaattaagtcaaattgaggagttgcaaaaacggattgtttcgctcaaagatttcattaaccgcactgctaaagaagtagctgaaaaactggaaattactaagattctccacaagcgccccgctaatatttctgggggtcaacaacaacgggtagctattgctagagcaattgttagaagacctaaggttctacttatggacgaaccgttgtccaacttagatgcgaagttgcgggtacaaacacggcaatggatccgtaagttccagcaagacctgcaaattactactgtctttgtgactcacgatcaggaagaggcaatgagtattagcgacacgattgtctgcatgtcaactggtaaggtacagcaaataggctccccttccgagttgtacttaaaaccggccaatgagttcgtcgctacctttttgggtagcccggaaatgaatatcgtcaacgctacggttaaagctggacagctgttgtgaaacgaaaatcccctagttaaaacaaagtttgatttacctgatggtgccattcgggttggtttccgttatgatgaggtaacggctcccaaaaacgatggtagtcccgtatttagcggtacccttataagtgtggaaaacctagggaaacacatggttggggttgtggagagcaatggggtacagctcaatgtgcgcttagaattgagccaccagtttgaagtaggtaatgcagttaagtttacgattaagcctaatggcttacacttctttgatccccaaacaacgcaacgggtggaggtaaaacatgtttaa

SEQ ID NO: 111 - DNA, 360 bp, Mycoplasma pneumoniae
atgaagttaccacctcgaagaaagcttaaactgtttttattagcctgaatgctagtgctgttcagcgctttaatagtgcttgcaaccttaattttggtacagcacaacaataccgaactgacagaagttaagagtgaattgagtccccttaacgttgttttacacgcagaagaggatacagtacaaattcagggcaagccgattactgagcaagcatggtttattcctacagttgctggttgctttggttttagtgccctagccatcatcttgggccttgctataggactgccaattgtgaagcgcaaggaaaaacgcttattggaggaaaaggaacgccaagaacagttagcggaacag

(19) United States
(12) Patent Application Publication
(10) Pub. No.: US 2023/0310564 A1
(43) Pub. Date: Oct. 5, 2023

(54) Attenuated Mycoplasma Bacteria

(71) Applicants: FUNDACIÓ CENTRE DE REGULACIÓ GENÒMICA, Barcelona (ES); INSTITUCIÓ CATALANA DE RECERCA I ESTUDIS AVANÇATS, Barcelona (ES)

(72) Inventors: Carlos Piñero Lambea, Madrid (ES); Maria Lluch Senar, Barcelona (ES); Luis Serrano Pubul, Barcelona (ES)

(21) Appl. No.: 17/912,331

(22) PCT Filed: Mar. 19, 2021
(86) PCT No.: PCT/EP2021/057122

§ 371 Date: Sep. 16, 2022

(30) Foreign Application Priority Data
Mar. 19, 2020 (EP) 20382207.7

(51) Int. Cl. A61K 39/02; C12N 1/20; C12N 1/36; C12N 15/74; C12N 9/04; C12N 9/90; C12N 9/10; A61P 31/04

(52) U.S. Cl. CPC A61K 39/0241; A61P 31/04; C12N 1/20; C12N 1/36; C12N 9/0006; C12N 9/1051; C12N 9/90; C12N 15/74; C12Y 101/05003; C12Y 501/03002; A61K 2039/522; C12N 2800/101

(57) ABSTRACT

The present invention concerns genetically modified Mycoplasma bacteria. Also intended are methods of generating attenuated Mycoplasma bacteria and their use to produce heterologous gene products. Further intended are pharmaceutical compositions comprising the attenuated Mycoplasma bacteria described herein.

### CROSS-REFERENCE TO RELATED APPLICATIONS

This application is a national phase entry under 35 U.S.C. § 371 of International Patent Application PCT/EP2021/057122, filed Mar. 19, 2021, designating the United States of America and published in English as International Patent Publication WO 2021/186052 on Sep. 23, 2021, which claims the benefit under Article 8 of the Patent Cooperation Treaty to European Patent Application Serial No. 20382207.7, filed Mar. 19, 2020, the entireties of which are hereby incorporated by reference.

### FIELD OF THE INVENTION

The invention relates to the field of molecular biology, more specifically the field of genome engineering and synthetic biology. Aspects of the invention relate to attenuated Mycoplasma bacteria.

### BACKGROUND OF THE INVENTION

Recently, interest has (re)sparked in using live organisms, especially bacteria, as delivery vehicles to introduce all sorts of cargo into a host organism (Akin et al., Bacteria-mediated delivery of nanoparticles and cargo into cells, Nature Nanotechnology, 2007). Several organisms have been proposed to function as such a delivery vehicle including Escherichia coli or Saccharomyces cerevisiae. These organisms have been traditionally preferred due to their ease of growth in laboratory conditions and high recombination efficiencies. High recombination efficiency is often a key determining factor to select a suitable delivery bacterium since it is crucial that these bacteria are not pathogenic for the host organism, and usually genetic perturbations have to be introduced to said bacteria to reduce their toxicity or immunogenicity.

Due to the advances in the field of genome engineering, more bacterial species can now efficiently be engineered to serve as such a delivery vehicle. One genus of bacteria that particularly benefits from these advancements is the Mycoplasma genus. The Mycoplasma genus comprises a group of bacteria that has several peculiar traits, including the lack of a cell wall, a streamlined genome, and a variant genetic code in which the codon UGA is translated into tryptophan instead of a stop codon. (Razin et al., Molecular biology and pathogenicity of Mycoplasmas, Microbiology and Molecular Biology Reviews, 1998). These traits contribute to increased biosafety and limited horizontal gene transfer capabilities, both of which are commonly raised concerns in the technical field. Furthermore, in theory the small genome size of Mycoplasma bacteria is convenient for devising an optimal genetic framework that constitutes a reduced or minimal set of genes while still maintaining specific desired functions of Mycoplasma when used as delivery vehicle (Lluch-Senar et al., Defining a minimal cell: essentiality of small ORFs and ncRNAs in a genome-reduced bacterium, Molecular Systems Biology, 2015). However, to date no attenuated Mycoplasma strains have been described that show improved properties for this purpose. A thorough characterization of attenuated Mycoplasma strains is thus lacking. Nevertheless, such a characterization would enable further research to harness Mycoplasma bacteria for a multitude of medical applications, such as using the Mycoplasma as a production means for heterologous proteins inside the host organism at desired locations.

One Mycoplasma strain of particular interest is Mycoplasma pneumoniae. In addition to the traits stated above for the Mycoplasma genus in general, M. pneumoniae has a small genome of 816 kb, and has reduced metabolic and genetic networks, which reduce the risk of unwanted interference of any hypothetical engineered circuits. Additionally, M. pneumoniae is a mild pathogen that can be eliminated with available antibiotics and is one of the most thorough characterized bacteria. However, despite this deep understanding little is known about the physiology of M. pneumoniae. M. pneumoniae is the causative agent of atypical pneumonia and other extra-pulmonary pathologies in humans. The number of antimicrobial resistant infections is steadily rising (Beeton et al., Mycoplasma pneumoniae infections, 11 countries in Europe and Israel, 2011 to 2016, Eurosurveillance, 2020). An increasing number of studies have reported that the immune response of an infected organism acts as a double-edged sword, not only playing an antibacterial role in the early stages of infection but also causing tissue damage as a persistent effect in many types of bacterial infection. M. pneumoniae mice models have been established and studies directed to the correlation between immune response and lung injury revealed that immunosuppression reduces lung injury in these models (Shi et al., Immunosuppression reduces lung injury cause by Mycoplasma pneumoniae infection, Scientific Reports, 2019).

Nevertheless, an additional challenge when developing attenuated Mycoplasma strains, and in fact bacteria in general, lays in the observation that several genes encoding unwanted gene products (e.g. pathogenic determinants) are essential genes which cannot be readily removed (Lluch-Senar et al., Defining a minimal cell: essentiality of small ORFs and ncRNAs in a genome-reduced bacterium, Molecular Systems Biology, 2015). Hence, attenuated strains are often based on multiple genetic modifications that in combination achieve the desired properties. However, the outcome of combining several genetic modifications in Mycoplasma is unpredictable.

There is thus a clear yet unmet need for attenuated Mycoplasma (pneumoniae) strains that can be used as research tools and/or delivery vehicles of molecular cargo.

### SUMMARY OF THE INVENTION

Through extensive experimental optimization, the inventors have identified a set of genes, which can be removed or inactivated to obtain attenuated Mycoplasma strains that are still viable and capable of secreting active biological compounds when introducing in a host organism. As evidenced by the examples, which are illustrating certain representative embodiments of the invention, the present invention hence relates to attenuated Mycoplasma strains that comprise a combination of genetic mutations leading to a desired phenotype. Furthermore, the inventors describe several particularly interesting attenuated Mycoplasma strains for use as a medicine or a vaccine. The invention therefore caters to the unmet need for less pathogenic yet viable Mycoplasma strains.

The invention therefore provides the following aspects:

Aspect 1. A genetically modified Mycoplasma bacterium, wherein said Mycoplasma bacterium comprises in its genome a deletion, substitution, and/or insertion of one or more nucleotides in the gene or operon of Ca2+ dependent cytotoxic nuclease gene (MPN133) or an equivalent thereof and/or ADP-ribosyltransferase CARDS gene (MPN372) or an equivalent thereof, that reduces the pathogenicity and/or immunogenicity of said Mycoplasma bacterium compared to a reference Mycoplasma bacterium having an identical genomic sequence with the proviso that said reference Mycoplasma bacterium does not comprise said deletion, substitution, and/or insertion in one or more nucleotides of said operons.

Aspect 2. The genetically modified Mycoplasma bacterium according to aspect 1, wherein said bacterium further comprises a deletion, substitution, and/or insertion of one or more nucleotides in one or more genes or operons encoding a protein capable of eliciting Guillain-Barre in a host organism, preferably encoding UDP-glucose 4-epimerase (MPN257) or an equivalent thereof and/or glycosyltransferase (MPN483) or an equivalent thereof.

Aspect 3. A genetically modified Mycoplasma bacterium, wherein said bacterium comprises a functional modification in one or more genes or operons encoding a protein capable of eliciting Guillain-Barre, preferably wherein said protein is capable of eliciting Guillain-Barre by producing or assisting in producing immunogenic lipids in a host organism.

Aspect 4. The genetically modified Mycoplasma bacterium according to aspect 3, wherein said Mycoplasma bacterium comprises a deletion, substitution, and/or insertion of one or more nucleotides in an operon or gene encoding UDP-glucose 4-epimerase (MPN257) or an equivalent thereof and/or in a gene or operon encoding glycosyltransferase (MPN483) or an equivalent thereof.

Aspect 5. The genetically modified Mycoplasma bacterium according to any one of aspects 2 to 4, wherein said Mycoplasma bacterium comprises a deletion, substitution, and/or insertion of one or more nucleotides in an operon or gene encoding UDP-glucose 4-epimerase (MPN257) or an equivalent thereof and a substitution in an operon or gene encoding glycosyltransferase (MPN483) or equivalent thereof, preferably wherein the Mycoplasma bacterium comprises a deletion, substitution, and/or insertion of one or more nucleotides in an operon or gene encoding UDP-glucose 4-epimerase (MPN257) or an equivalent thereof and a substitution of a complete operon or complete gene encoding glycosyltransferase (MPN483) a functional fragment of said operon or said gene or said equivalent.

Aspect 6. The genetically modified Mycoplasma bacterium according to aspect 5, wherein the substitution of MPN483 or equivalent thereof is M. genitalium MG_517, M. agalactiae MAGA_RS00300, and/or B. subtilis ugtP.

Aspect 7. The genetically modified Mycoplasma bacterium according to any one of the preceding aspects, wherein said bacterium further comprises a deletion, substitution, and/or insertion in a gene or operon encoding a peroxide producing protein, preferably in a gene or operon encoding glycerol 3-phosphate oxidase, more preferably encoding glycerol-3-phospate dehydrogenase (MPN051) or an equivalent thereof.

Aspect 8. The genetically modified Mycoplasma bacterium according to any one of the preceding aspects, wherein said Mycoplasma bacterium is selected from the group consisting of: M. adleri, M. agalactiae, M. agassizii, M. alkalescens, M. alligatoris, M. alvi, M. amphoriforme, M. anatis, M. anseris, M. arginine, M. arthritidis, M. auris, M. bovigenitalium, M. bovirhinis, M. bovis, M. bovoculi, M. buccale, M. buteonis, M. californicum, M. canadense, M. canis, M. capricolum, M. capricolum subsp. capricolum, M. capricolum subsp. capripneumoniae, M. caviae, M. cavipharyngis, M. ciconiae, M. citelli, M. cloacale, M. collis, M. columbinasale, M. columbinum, M. columborale, M. conjunctivae, M. corogypsi, M. cottewii, M. cricetuli, M. crocodyli, M. cynos, M. dispar, M. edwardii, M. elephantis, M. equigenitalium, M. equirhinis, M. falconis, M. fastidiosum, M. faucium, M. felifaucium, M. feliminutum, M. felis, M. feriruminatoris, M. fermentans, M. flocculare, M. gallinaceum, M. gallinarum, M. gallisepticum, M. gallopavonis, M. gateae, M. genitalium, M. glycophilum, M. gypis, M. haemocanis, M. haemofelis, M. haemomuris, M. hominis, M. hyopharyngis, M. hyopneumoniae, M. hyorhinis, M. hyosynoviae, M. iguana, M. imitans, M. indiense, M. iners, M. iowae, M. lagogenitalium, M. leachii, M. leonicaptivi, M. leopharyngis, M. lipofaciens, M. lipophilum, M. maculosum, M. meleagridis, M. microti, M. moatsii, M. mobile, M. molare, M. mucosicanis, M. muris, M. mustelae, M. mycoides, M. mycoides subsp. capri, M. mycoides subsp. mycoides, M. neophronis, M. neurolyticum, M. opalescens, M. orale, M. ovipneumoniae, M. ovis, M. oxoniensis, M. penetrans, M. phocicerebrale, M. phocidae, M. phocirhinis, M. pirum, M. pneumoniae, M. primatum, M. pullorum, M. pulmonis, M. putrefaciens, M. salivarium, M. simbae, M. spermatophilum, M. spumans, M. sturni, M. sualvi, M. subdolum, M. suis, M. synoviae, M. testudineum, M. testudinis, M. tullyi, M. verecundum, M. wenyonii, M. yeatsii, and M. coccoides, preferably wherein said Mycoplasma bacterium is selected from the group consisting of: M. pneumoniae, M. genitalium, M. hyorhinis, M. bovis, M. agalactiae, M. gallisepticum, and M. feriruminatoris.

Aspect 9. The genetically modified Mycoplasma bacterium according to any one of the preceding claims, wherein said Mycoplasma bacterium is a M. pneumoniae bacterium, preferably M. pneumoniae M129-B7.

Aspect 10. The genetically modified Mycoplasma bacterium according to any one of the preceding aspects, wherein said bacterium further comprises a deletion, substitution, and/or insertion of one or more nucleotides in a gene or operon encoding a second (surface) nuclease, preferably encoding membrane nuclease A (MPN491) or an equivalent thereof.

Aspect 11. The genetically modified Mycoplasma bacterium according to any one of the preceding aspects, wherein said bacterium comprises a deletion, substitution, and/or insertion of one or more nucleotides in one or more genes or operons encoding for a cytoadherence protein, preferably selected from the group consisting of: MPN141, MPN142, MPN453, MPN447, MPN309, MPN310, and MPN452, or an equivalent of any one of MPN 141, MPN142, MPN453, MPN447, MPN309, MPN310, and MPN452.

Aspect 12. The genetically modified Mycoplasma bacterium according any one of the preceding aspects, wherein said bacterium further comprises a deletion, substitution, and/or insertion of one or more nucleotides in a gene or operon encoding an immunogenic protein that is capable of eliciting an immune response in a host organism, preferably in a gene or operon encoding conserved hypothetical protein MPN_400 (MPN400) or an equivalent thereof.

Aspect 13. The genetically modified Mycoplasma bacterium according to any one of the preceding aspects, wherein said bacterium further comprises a deletion, substitution, and/or insertion of one or more nucleotides in a gene or operon encoding a protein that inhibits growth of said bacterium in a bioreactor, preferably encoding chaperone protein YajL (MPN294) or an equivalent thereof.

Aspect 14. The genetically modified Mycoplasma bacterium according to any one of the preceding aspects, wherein said bacterium further comprises a deletion, substitution, and/or insertion of one or more nucleotides in one or more genes or operons encoding a lipoprotein, preferably selected from the group consisting of: MPN141, MPN142, MPN152, MPN162, MPN199, MPN200, MPN224, MPN233, MPN271, MPN284, MPN288, MPN293, MPN333, MPN372, MPN415, MPN447, MPN592, MPN597, MPN602, MPN611, MPN011, MPN052, MPN054, MPN058, MPN083, MPN084, MPN097, MPN098, MPN363, MPN369, MPN408, MPN411, MPN436, MPN439, MPN442, MPN444, MPN456, MPN467, MPN489, MPN506, MPN523, MPN582, MPN585, MPN586, MPN587, MPN588, MPN590, MPN591, MPN592, MPN639, MPN640, MPN641, MPN642, MPN643, MPN644, MPN645, MPN646, MPN647, MPN648, MPN649, MPN650, MPN654, or their equivalents.

Aspect 15. The genetically modified Mycoplasma bacterium according to any one of the preceding aspects, wherein said bacterium comprises a deletion, substitution, and/or insertion of one or more nucleotides in a prolipoprotein diacylglyceryl transferase and a prolipoprotein signal peptidase, preferably in prolipoprotein diacylglyceryl transferase gene MPN224 and lipoprotein signal peptidase gene MPN293 or their operons or equivalents of MPN224 and MPN293.

Aspect 16. The genetically modified Mycoplasma bacterium according to any one of the preceding aspects, wherein said bacterium further comprises a deletion, substitution, and/or insertion of one or more nucleotides in a gene or operon encoding an oncogenic protein, preferably encoding high affinity transport system protein p37 (MPN415) or an equivalent thereof.

Aspect 17. The genetically modified Mycoplasma bacterium according to any one of the preceding aspects, wherein said bacterium further comprises a deletion, substitution, and/or insertion of one or more nucleotides in a gene or operon encoding an RNA polymerase factor, preferably encoding probable RNA polymerase sigma-D factor (MPN626) or an equivalent thereof.

Aspect 18. The genetically modified Mycoplasma bacterium according to any one of the preceding aspects, wherein said bacterium further comprises a deletion, substitution, and/or insertion of one or more nucleotides in one or more genes or operons encoding a secreted Mycoplasma gene product, preferably selected from the group consisting of: MPN400, MPN036, MPN592, MPN509, MPN647, MPN084, MPN625, MPN213, MPN489, MPN142, MPN444, MPN642, MPN398, MPN491, MPN083, MPN141, or their equivalents.

Aspect 19. The genetically modified Mycoplasma bacterium according to any one of the preceding aspects, wherein the reduced pathogenicity and/or immunogenicity is characterized by a reduction of toxicity by at least 30%, preferably at least 50%, more preferably at least 75%, most preferably at least 90%, when said bacterium is introduced to a host organism, preferably introduced in the respiratory system of said host organism, when compared to a reference Mycoplasma bacterium, wherein said reference Mycoplasma bacterium has an identical genomic sequence with the proviso that said reference Mycoplasma bacterium does not comprise said deletions, substitutions, and/or insertions of one or more nucleotides in its genomic sequence, preferably wherein the reference Mycoplasma bacterium is M. pneumoniae M129-B7.

Aspect 20. The genetically modified Mycoplasma bacterium according to aspect 19, wherein the reduction of toxicity is assessed by measuring the inflammatory response in the lung, preferably by measuring inflammatory cytokines, measuring pulmonary lesions, and/or measuring hemorrhagic lesions in the mammary gland and/or lung.

Aspect 21. The genetically modified Mycoplasma bacterium according to any one of the preceding aspects, wherein said Mycoplasma bacterium comprises a nucleotide sequence encoding an exogenous gene product or a functional fragment thereof, preferably wherein said nucleotide sequence is comprised in the genomic sequence of said Mycoplasma bacterium.

Aspect 22. The genetically modified Mycoplasma bacterium according to aspect 21, wherein said exogenous gene product or functional fragment thereof is a protein, preferably a therapeutic protein, a protein involved in specific attachment to a host protein, an enzyme, immunogenic protein, or DNA-binding protein, more preferably wherein said (therapeutic or immunogenic protein) is expressed on the surface of said Mycoplasma bacterium and/or is secreted by said Mycoplasma bacterium.

Aspect 23. The genetically modified Mycoplasma bacterium according to any one of the preceding aspects, wherein said bacterium is obtained by introducing said deletions, substitutions, and/or insertions of one or more nucleotides in one or more genes or operons of a Mycoplasma bacterium genome by random transposon insertion or recombinant DNA technology, preferably by a genome engineering method, more preferably by a recombinase and/or nuclease-based genome engineering method.

Aspect 24. The genetically modified Mycoplasma bacterium according to any one of the preceding aspects, wherein said genome comprising one or more deletions, substitutions, and/or insertions of one or more nucleotides is partially, preferably completely obtained by chemical synthesis.

Aspect 25. A Mycoplasma bacterium according to any one of aspects 1 to 24, for use as a medicament.

Aspect 26. A Mycoplasma bacterium according to any one of aspects 1 to 24, for use as a vaccine.

Aspect 27. The Mycoplasma bacterium for use according to aspect 26, wherein said Mycoplasma bacterium displays at least one, preferably at least two, more preferably at least three distinct exogenous proteogenic sequences on its surface.

Aspect 28. The Mycoplasma bacterium for use according to aspect 27, wherein the at least one exogenous proteogenic sequence is an exogenous antigenic sequence.

Aspect 29. A Mycoplasma bacterium according to any one of aspects 1 to 24, for use to modulate the composition of a lung microbiome in a subject.

Aspect 30. A method of producing an attenuated Mycoplasma bacterium, wherein the method comprises introducing a deletion, substitution, and/or insertion of one or more nucleotides in at least two genes or operons encoding a gene product independently selected from the group consisting of: cytoadherence proteins, lipid synthesis enzymes producing immunogenic products, oxidoreductases, nucleases, toxins, lipoproteins, inflammatory regulating proteins, immunogenic proteins, or cancer inducing proteins.

Aspect 31. The method according to aspect 30, wherein said functional modifications are introduced in a live Mycoplasma bacterium, via random transposon insertion, or with a site-directed recombinase and/or a site-directed nuclease.

Aspect 32. The method according to aspect 30 or 31, wherein the method comprises a step of providing a synthetic genome, or a portion thereof, and transferring said (portion of) the synthetic genome to a naturally occurring Mycoplasma bacterium.

Aspect 33. The method according to aspect 32, wherein the method further comprises a step of inactivating, preferably degrading, and/or removing the original genome of said live Mycoplasma bacterium.

Aspect 34. Use of an attenuated Mycoplasma bacterium of any of aspects 1 to 24 for the production of at least one exogenous gene product or fragment thereof.

Aspect 35. A pharmaceutical composition comprising the genetically modified Mycoplasma bacterium according to any one of aspects 1 to 24.

Aspect 36. The genetically modified Mycoplasma bacterium according to any one of aspects 1 to 24, further comprising a deletion, substitution, and/or insertion of one or more nucleotides in at least one gene or operon, preferably at least two operons or genes selected from the group consisting of Table 1.

Aspect 37. A kit of part comprising the genetically modified Mycoplasma according to any one of aspects 1 to 24.

Aspect 38. A method of treating a disease using a genetically modified Mycoplasma bacterium as according to any one of aspects 1 to 24.

Aspect 39. Use of a genetically modified Mycoplasma bacterium according to any one of aspects 1 to 24, for the manufacture of a medicament.

Aspect 40. A genetically modified Mycoplasma bacterium comprising the P30 exposure sequence as disclosed herein, preferably wherein the P30 exposure sequence is comprised as part of a heterologous gene inserted in the Mycoplasma bacterium.

### BRIEF DESCRIPTION OF THE FIGURES

FIG. 1. Quantitative analysis of attachment by qPCR. Percentage of cells attached and detached to the plastic after 24 h of incubation was calculated after genomic DNA extraction and quantification by qPCR. 99% of CV3 and CV19 cells are detached from the plastic surface. Thus, these strains display a non-adherent phenotype.

FIG. 2. Western Blot to detect protein exposition in different variants of the chassis. Isolated membrane and cytoplasm fractions of the total protein extract served as input material. The heterologous protein (here a nanobody against P. aeruginosa labelled with a Flag-tag) was detected by using specific antibody against the tag sequence. Top panel: Ponceau staining, lower panel: anti-flag detection. Left portion indicates membrane fraction, right portion cytoplasmic fraction. Lane 1: Wild-type (WT), lane 2: WT + nanobody-Flag; lane 3: CV19, lane 4: CV19 + nanobody-Flag. In lane 4, which corresponds to the membrane fraction of the CV19 strain transformed with the Nanobody, the anti-flag signal shows a higher intensity than WT Mycoplasma transformed with the same platform (lane 2). RL7: Ribosomal protein.

FIG. 3. Western Blot to detect the expression of the SH3 chimera, SH3 and Nanobody in the CV2 strain. Lane 1 and 2: Mycoplasma not expressing any heterologous protein(s). Lanes 3, 4, 5: expression of SH3 chimera, SH3, and Nanobody in the CV2 strain.

FIG. 4. Western blot showing P97 expression in different Mycoplasma strains. Lane 1: WT Mycoplasma, lane 2: WT_P97-lyso-disp, lane 3: CV2_Lyso-Disp-P97, lane 4: CV3deltaCm-P97, lane 5: CV3_Lyso-Disp, lane 6: CV3_Lyso-Disp-Vsa, lane 7: CV3deltaCm-P97-Lyso-Disp, lane 8: positive control: L-D: Disp-TEV-Lyso Fusion, lane 9: positive control: CV2-Ag18.2#3.

FIG. 5. Part of M. pneumoniae metabolism showing the production of peroxide by the GlpD enzyme (as described in detail by Lluch et al., Comparative “-omics” in Mycoplasma pneumoniae Clinical Isolates Reveals Key Virulence Factors, PLOS one, 2015).

FIG. 6. Peroxide production of the CV2 strain (ΔCARDS, ΔMPN133) compared to the CV8 chassis (CV2 ΔGlpD, ΔCARDS, ΔMPN133). Methods as indicated in Hames et al., Glycerol metabolism is important for cytotoxicity of Mycoplasma pneumoniae, Journal of Bacteriology, 2009.

FIGS. 7A and 7B. (FIG. 7A) Study of haemorrhagic lesions in mammary gland tissue after 4 days of infection with different mycoplasma strains. (FIG. 7B) Number of Mycoplasma bacteria present in the tissue after 4 days of infection by quantification of colonies formed on agar plates. 6 animals were infected for each condition with 10⁸ colony forming units (CFU).

FIG. 8. Lipoprotein processing in M. pneumoniae.

FIG. 9. Galactose metabolism in M. pneumoniae (Adapted from Klement et al., A processive lipid glycosyltransferase in the small human pathogen Mycoplasma pneumoniae: involvement in host immune response, Molecular Microbiology, 2007). Metabolic pathways for the synthesis of glycolipids and phospholipids in M. pneumoniae. GP, glycerol-phosphate; 1aG3P 1 acyl glycerolphosphate; 1,2 aG3P, 1,2 dyacilglycerol phosphate; DAG, diacylglycerol; MH, monohexose; DH, dihexose; TH, trihexose; PG, phosphatidylglycerol; APG, acyl-phosphatidylglycerol; CL, cardiolipin; Cer, ceramide. G6P glucose-6-phosphate; G1P glucose -1-phosphate; UDP-Glc uracyldiphosphate-glucose; UDP-Gal uracyldiphosphate-galactose. Unlabelled arrows denote non-identified/annotated enzymes.

FIG. 10. Macroscopic lesions of the CD1 mice mammary glands infected by different Mycoplasma pneumoniae chassis. CV2 complemented with nuclease ofM. genitalium (CV7), WTΔp30 (CV16), and WT ΔMPN051 is a transposon mutant (CV30).

FIG. 11. Analysis of the persistence of the different strains used to infect the CD1 mice mammary glands. The CV2GM strain corresponds to CV7, and the WTKO MPN133 strain to CV31. Similar persistence can be observed for each strain.

FIG. 12. Analysis of the persistence of the different strains used to infect the CD1 mice mammary glands. The CV2Δglp::gpsA strains corresponds to CV8, WTΔp30 strain to CV16. Similar persistence can be observed for each strain.

FIG. 13. Infection in CD1 mice with WT and CV2 strains. lungs from CD1 mice infected with WT and CV2 chassis at day 2 and day 4. At two days no macroscopic lesions are visible. At day 4 some pneumonia regions of diffuse character are more abundant in the WT strains than in the CV2 strain.

FIG. 14. Histopathology sections of the CD1, lung mice infected with Mycoplasma WT, CV2 and CV8 chassis. From top to bottom, Control, CV8, CV2 and WT and from left to right 2 days post infection and 4 days post infection.

FIG. 15. Evolution of mice histopathology lesions at 2 (top panel) and 4 (bottom panel) days post infection in MR28->Control, MR27->CV8, MR22 (2)->CV2, MR2(1)->WT. Higher values indicate more lesions. It can be observed from the graph that the CV8 strain behaves similarly to the control (i.e. inoculation of mice lung with PBS).

FIG. 16. CFUs of the WT, CV2 and CV8 chassis 2 and 4 days post infection of CD1 mice lungs.

FIG. 17. CFUs of Mycoplasma cells obtained from lung tissue 2 days and 4 days post infection with the WT, CV2, CV30 (WT_tn051) and CV16 (WTDp30) strains in mice CD1. Inoculation rates: WT: 3.9×10⁷ CFU, CV2: 0.3×10⁷ CFU, WT Tn::glpD: 3.9×10⁷ CFU, WT Δp30: 0.15×10⁷ CFU.

FIG. 18. Comparison between CD1 mouse model and BALBc mouse model. In CD1 mice the Mycoplasma strains show a decrease of two orders of log10 after two days of infection whilst in BALBc model the decrease is less than 1 order for certain strains. WT_Tn (CV30), WTDp30 (CV16). Inoculation rates CD1 model: WT: 1.3×10⁷ CFU, WT Tn::glpD: 0.87 ×10⁷ CFU, WT Δp30: 0.5×10⁷CFU. Inoculation rates BALBc model: WT: 1.3×10⁷ CFU, CV2: 0.31×10⁷ CFU, CV8: 0.58×10⁷ CFU.

FIGS. 19A-19K. Nucleotide and amino acid sequences to which reference is made throughout the present disclosure.

FIG. 20. Representation of the results of a colorimetric assay sensitive to pH changes in the medium due to lactate and acetate production of growing M. pneumoniae. Filled upper line: WT; middle line (not filled, circular); MPN257 KO; bottom line (not filled, rectangular symbols): MPN 483 KO.

FIGS. 21A-21C. Growth curves of the strains wherein MPN483 is replaced by other glycosiltransferases. FIG. 21A) WT M. pneumoniae (empty symbols), MG_517 (filled symbols). FIG. 21B) WT M. pneumoniae (empty symbols), MAGA_RS00300 (filled symbols). FIG. 21C) WT M. pneumoniae (empty symbols), ugtP (filled symbols).

FIGS. 22A-22J. Mass spectroscopy analysis of different M. pneumoniae strains. Δ483 is the deletion of MPN483. Δ 257 is the deletion of MPN257; Δ 483 9T is the deletion of MPN483 and the replacement by MG_517; Δ MPN483 AG is the deletion of MPN483 and the replacement by MAGA RS00300; Δ MPN483 SUB is the replacement of MPN483 by the UgtP from B. subtilis. For all the strains two independent lipid extractions were analyzed (A and B) Cer’s: ceramides. SM’s: sphingomyelin. Cer monohexoides (CMH’s): glucoceramide (GluCer’s) and galactoceramide (GalCer’s). LacCer’s: Ceramide dihexosides (CDH’s). Samples A and B indicate technical replicates. Certain strains were analysed twice (FIGS. 22A and 22B) as biological replicates.

FIG. 23. Dot blot Western to assess galactocerebrosides and glycosylceramide presence in extracts of distinct M. pneumoniae strains.

FIGS. 24A-24C. Impact of the individual gene knock outs on the lesions that can be observed after infection. It can be observed that CV2 (i.e. the double MPN133 and MPN372 knockout) shows little to no lesions (FIG. 24A) despite being present at similar CFUs (FIG. 24C). FIG. 24B: typical lesion morphology induced by a Mycoplasma pneumoniae infection.

FIG. 25. Time course experiment indicating in vivo replication and clearance of WT Mycoplasma pneumoniae (left panel) and CV (right panel).

### DETAILED DESCRIPTION OF THE INVENTION

As used herein, the singular forms “a”, “an”, and “the” include both singular and plural referents unless the context clearly dictates otherwise.

The terms “comprising”, “comprises” and “comprised of” as used herein are synonymous with “including”, “includes” or “containing”, “contains”, and are inclusive or open-ended and do not exclude additional, non-recited members, elements or method steps. The terms also encompass “consisting of” and “consisting essentially of”, which enjoy well-established meanings in patent terminology.

The recitation of numerical ranges by endpoints includes all numbers and fractions subsumed within the respective ranges, as well as the recited endpoints. This applies to numerical ranges irrespective of whether they are introduced by the expression “from... to...” or the expression “between... and...” or another expression.

The terms “about” or “approximately” as used herein when referring to a measurable value such as a parameter, an amount, a temporal duration, and the like, are meant to encompass variations of and from the specified value, such as variations of +/-10% or less, preferably +/-5% or less, more preferably +/-1% or less, and still more preferably +/-0.1% or less of and from the specified value, insofar such variations are appropriate to perform in the disclosed invention. It is to be understood that the value to which the modifier “about” or “approximately” refers is itself also specifically, and preferably, disclosed.

Whereas the terms “one or more” or “at least one”, such as one or more members or at least one member of a group of members, is clear per se, by means of further exemplification, the term encompasses inter alia a reference to any one of said members, or to any two or more of said members, such as, e.g., any ≥3, ≥4, ≥5, ≥6 or ≥7 etc. of said members, and up to all said members. In another example, “one or more” or “at least one” may refer to 1, 2, 3, 4, 5, 6, 7 or more.

The discussion of the background to the invention herein is included to explain the context of the invention. This is not to be taken as an admission that any of the material referred to was published, known, or part of the common general knowledge in any country as of the priority date of any of the claims.

Throughout this disclosure, various publications, patents and published patent specifications are referenced by an identifying citation. All documents cited in the present specification are hereby incorporated by reference in their entirety. In particular, the teachings or sections of such documents herein specifically referred to are incorporated by reference.

Unless otherwise defined, all terms used in disclosing the invention, including technical and scientific terms, have the meaning as commonly understood by one of ordinary skill in the art to which this invention belongs. By means of further guidance, term definitions are included to better appreciate the teaching of the invention. When specific terms are defined in connection with a particular aspect of the invention or a particular embodiment of the invention, such connotation or meaning is meant to apply throughout this specification, i.e., also in the context of other aspects or embodiments of the invention, unless otherwise defined. For example, embodiments directed to products are also applicable to corresponding features of methods and uses.

In the following passages, different aspects or embodiments of the invention are defined in more detail. Each aspect or embodiment so defined may be combined with any other aspect(s) or embodiment(s) unless clearly indicated to the contrary. In particular, any feature indicated as being preferred or advantageous may be combined with any other feature or features indicated as being preferred or advantageous.

Reference throughout this specification to “one embodiment”, “an embodiment” means that a particular feature, structure or characteristic described in connection with the embodiment is included in at least one embodiment of the present invention. Thus, appearances of the phrases “in one embodiment” or “in an embodiment” in various places throughout this specification are not necessarily all referring to the same embodiment, but may. Furthermore, the particular features, structures or characteristics may be combined in any suitable manner, as would be apparent to a person skilled in the art from this disclosure, in one or more embodiments. Furthermore, while some embodiments described herein include some but not other features included in other embodiments, combinations of features of different embodiments are meant to be within the scope of the invention, and form different embodiments, as would be understood by those in the art. For example, in the appended claims, alternative combinations of claimed embodiments are encompassed, as would be understood by those in the art.

Amino acids are referred to herein with their full name, their three-letter abbreviation or their one letter abbreviation.

“Mycoplasma”, “Mycoplasma bacteria”, or “Mycoplasmas” as used interchangeably herein refers to the mollicute genus Mycoplasma which is characterized by lack of a cell wall around their cell membranes. Therefore, the plasma membrane forms the outer boundary of the Mycoplasma bacterial cell. Due to the absence of a cell wall, Mycoplasma has been found to have versatile shapes ranging from round to oblong, and display pleomorphism. “Pleomorphism” as used herein is a term used in histology and cytopathology to describe cells and/or their nuclei that may contain variable sizes, shape and staining. Culturable Mycoplasma species typically form small umbonate colonies on agar. The exact shape of the Mycoplasmas may depend on numerous parameters including osmotic pressure, nutritional quality of the culture medium, and growth phase. Certain Mycoplasma bacteria may be filamentous in their early and exponential growth phases or when attached to surfaces or other cells. The filamentous form may be transitory, and in certain conditions the filaments may branch or fragment into chains of cocci or individual vegetative cells. Alternative species are typically coccoid and do not develop a filamentous phase. Certain species develop specialized attachment tip structures involved in the process of colonization and/or contribute to virulence. Mycoplasma bacteria comprise 16S and 70S type ribosomes and a replicating disc to assist the replication process, and isolation of the genetic material. Mycoplasma bacteria may either live as saprophytes or more commonly as parasites. The term “saprophytes” refers to the chemoheterotrophic extracellular digestion that takes place in the processing of decayed organic matter. Mycoplasma bacteria are commonly described as one of the smallest and simplest self-replicating organisms known to date. Naturally occurring Mycoplasma genomes vary from about 500 kilobases (kb) to 1500 kb and GC contents between 23-41 mole percent (mol%) have been described.

Techniques for enrichment and/or isolation of Mycoplasma bacteria from humans, various species of animals, and cell cultures have been extensively described in the art and are well-known to a skilled person (Tully and Razin, Molecular and diagnostic procedures in Mycoplasmology, Vol. 2, 1996). A skilled person is also aware that minimal standards for descriptions of new species have been outlined (Brown et al., Revise standards for description of new species of the class Mollicutes (division Tenericutes), International Journal of Systematic and Evolutionary Microbiology, 2007).

A substantial amount of Mycoplasma species have been described and include the non-exhaustive list of M. adleri, M. agalactiae, M. agassizii, M. alkalescens, M. alligatoris, M. alvi, M. amphoriforme, M. anatis, M. anseris, M. arginine, M. arthritidis, M. auris, M. bovigenitalium, M. bovirhinis, M. bovis, M. bovoculi, M. buccale, M. buteonis, M. californicum, M. canadense, M. canis, M. capricolum, M. capricolum subsp. capricolum, M. capricolum subsp. capripneumoniae, M. caviae, M. cavipharyngis, M. ciconiae, M. citelli, M. cloacale, M. collis, M. columbinasale, M. columbinum, M. columborale, M. conjunctivae, M. corogypsi, M. cottewii, M. cricetuli, M. crocodyli, M. cynos, M. dispar, M. edwardii, M. elephantis, M. equigenitalium, M. equirhinis, M. falconis, M. fastidiosum, M. faucium, M. felifaucium, M. feliminutum, M. felis, M. feriruminatoris, M. fermentans, M. flocculare, M. gallinaceum, M. gallinarum, M. gallisepticum, M. gallopavonis, M. gateae, M. genitalium, M. glycophilum, M. gypis, M. haemocanis, M. haemofelis, M. haemomuris, M. hominis, M. hyopharyngis, M. hyopneumoniae, M. hyorhinis, M. hyosynoviae, M. iguana, M. imitans, M. indiense, M. iners, M. iowae, M. lagogenitalium, M. leachii, M. leonicaptivi, M. leopharyngis, M. lipofaciens, M. lipophilum, M. maculosum, M. meleagridis, M. microti, M. moatsii, M. mobile, M. molare, M. mucosicanis, M. muris, M. mustelae, M. mycoides, M. mycoides subsp. capri, M. mycoides subsp. mycoides, M. neophronis, M. neurolyticum, M. opalescens, M. orale, M. ovipneumoniae, M. ovis, M. oxoniensis, M. penetrans, M. phocicerebrale, M. phocidae, M. phocirhinis, M. pirum, M. pneumoniae, M. primatum, M. pullorum, M. pulmonis, M. putrefaciens, M. salivarium, M. simbae, M. spermatophilum, M. spumans, M. sturni, M. sualvi, M. subdolum, M. suis, M. synoviae, M. testudineum, M. testudinis, M. tullyi, M. verecundum, M. wenyonii, M. yeatsii, M. coccoides. When the term Mycoplasma is used herein, this includes the non-limiting list of candidate species Moeniiplasma glomeromycotorum, M. aoti, M. corallicola, M. erythrocervae, M. girerdii, M. haematoparvum, M. haemobos, M. haemocervae, M. haemodidelphidis, M. haemohominis, M. haemolamae, M. haemomacaque, M. haemomeles, M. haemominutum, M. haemomuris subsp. musculi, M. haemomuris subsp. ratti, M. haemovis, M. haemozalophi, M. kahaneii, M. ravipulmonis, M. struthiolus, M. turicensis, M. haemotarandirangiferis, M. preputii and others such as M. insons, M. sphenisci, M. vulturis, and M. zalophi. Furthermore, it is evident to a skilled person that the term Mycoplasma additionally includes any Mycoplasma strain or species that is generated by genetic or chemical synthesis, or any sort of rational design and/or the reorganization of a naturally occurring Mycoplasma genomic sequence and that the term therefore also covers those Mycoplasma strains and species that are termed “synthetic Mycoplasma”, alternatively “Mycoplasma laboratorium”, “Mycoplasma synthia”, or even short “Synthia” in the art (Gibson et al., Creation of a bacterial cell controlled by a chemically synthesized genome, Science, 2010). Hence, in certain embodiments described throughout this specification, the Mycoplasma species subject of the invention have as genomic sequence a sequences comprising at least 30%, at least 35%, at least 40%, at least 45%, at least 50%, at least 55%, at least 60%, at least 65%, at least 70%, at least 75%, at least 80%, at least 85%, at least 90%, at least 95%, at least 97% global sequence identity to a naturally occurring Mycoplasma bacterium.

Preferred Mycoplasma bacteria in the context of the present invention include Mycoplasma selected from the group consisting of: M. pneumoniae, M. genitalium, M. hyorhinis, M. bovis, M. agalactiae, M. gallisepticum, and M. feriruminatoris.

Throughout the present disclosure “reference Mycoplasma bacteria” are mentioned. A skilled person readily appreciates that in the context of the present invention, such references and synonyms thereof intend to specify a Mycoplasma bacterium identical to the genetically modified Mycoplasma bacteria that is described in the relevant embodiment, which is identical or is considered identical by a skilled person based to the described Mycoplasma bacterium, with the proviso that the reference Mycoplasma bacterium does not comprise the specific genomic modifications described herein. Hence, it is further evident that a suitable reference Mycoplasma bacterium is of the same species as the genetically modified bacterium. For example, an isolated (not-genetically modified) M. pneumoniae is considered a suitable reference bacterium for a genetically modified M. pneumoniae bacterium according to the invention; an isolated (not-genetically modified) M. bovis bacterium is a suitable reference bacterium for a genetically modified M. bovis bacterium according to the invention; an isolated (not-genetically modified) M. genitalium bacterium is a suitable reference bacterium for a genetically modified M. genitalium bacterium according to the invention; etc. Such reference bacteria may be a subculture of the Mycoplasma culture that is separated from the actual culture that will serve as a starting population for the genome engineering process. Alternatively, suitable reference Mycoplasma bacteria may be obtained from commercial providers, such as the American Tissue Culture Collection (ATCC). Particularly in the context of M. pneumoniae, a preferred reference M. pneumoniae strain is the ATCC M129-B7 strain (ATCC identifier 29342).

Methods and tools to verify sequence homology or sequence identity between different sequences of amino acids or nucleic acids are well known to a person skilled in the art and include non-limiting tools such as Protein BLAST, ClustalW2, SIM alignment tool, TranslatorX and T-COFFEE. The percentage of identity between two sequences may show minor variability depending on the algorithm choice and parameters. The term “sequence identity” refers to the relationship between sequences at the nucleotide (or amino acid) level. The expression “% identical” is determined by comparing optimally aligned sequences, e.g. two or more, over a comparison window wherein the portion of the sequence in the comparison window may comprise insertions or deletions as compared to the reference sequence for optimal alignment of the sequences. The reference sequence does not comprise insertions or deletions.

A reference window is chosen and the “% identity” is then calculated by determining the number of nucleotides (or amino acids) that are identical between the sequences in the window, dividing the number of identical nucleotides (or amino acids) by the number of nucleotides (or amino acids) in the window and multiplying by 100. Unless indicated otherwise, the sequence identity is calculated over the whole length of the reference sequence.

The intended Mycoplasma genes are indicated throughout this specification by their MPN (M. pneumoniae) number. A skilled person is aware that this MPN nomenclature is a standard manner of gene annotation in the technical field and that gene and/or protein names are readily derivable from publicly available resources such as the M. pneumoniae database http://mympn.crg.eu/essentiality.php or (academic) publications (including but not limited to Lluch-Senar et al., Defining a minimal cell: essentiality of small ORFs and ncRNAs in a genome-reduced bacterium, Molecular Systems Biology, 2015). It is evident that the MPN numbers are intended to cover Mycoplasma genes from different strains, including the non-limited exemplary strains indicated above. Albeit the preferred method of identifying the position of the targeted genomic perturbations throughout the present disclosure is by means of the above-mentioned MPN references, it is evident for a skilled person that alternative annotations and classifications may be used to specify the same, or essentially the same genes. For example, a commonly used yet non-limiting system to annotate certain gene product is the IUBMB enzyme nomenclature. By means of illustration and not limitation, MPN483 would classify as an enzyme falling under IUBMB EC 2.4.1.47, since MPN483 can be considered a glycosyltransferase. Similarly, MPN257 would classify as an enzyme falling under IUBMB EC 5.1.3.2, since MPN257 can be considered a epimerase. Reference works and tools to link certain enzymatic activities to specific IUBMB EC numbers are readily available in the art (e.g. McDonald et al., ExplorEnz: the primary source of the IUBMB enzyme list, Nucleic Acids Research, 2009). Hence, when reference herein is made to a certain MPN number, such references also encompass the corresponding enzymes in orthologue Mycoplasma bacteria categorized under the same IUBMB EC number. Furthermore, it is evident that Mycoplasma bacteria not comprising the MPN gene as such are also envisaged. In these embodiments, functional modification of an equivalent gene for said species is envisaged.

Additionally, means and methods to express proteins, for inhibiting protein expression, the construction of suitable expression vectors, and methods contributing to the establishment of any biological framework comprising cells that mediate expression of one or more genes encoded in such expression vectors, any many additional techniques routinely used in molecular biology are known to a skilled person and have been described in the art on numerous occasions (e.g. Green and Sambrook et al., Molecular Cloning: A Laboratory Manual, 4th Ed., Cold Spring Harbor Laboratory Press, 2012). Methods to transform Mycoplasma bacteria, albeit historically a more difficult to transform genus of bacteria, have also been described in the art (Minion and Kapke, Transformation of Mycoplasmas, Mycoplasma protocols, 1998).

Methods and protocols to introduce nucleotide arrangements into bacteria, i.e. methods of bacterial transformation, are known to a person skilled in the art (Johnston et al., Bacterial transformation: distribution, shared mechanisms and divergent control, Nature reviews Microbiology, 2014). The term “transformation” is indicative for a genetic alteration of a cell resulting from the direct uptake and incorporation of exogenous genetic material. Transformation is a horizontal gene transfer process and is commonly used in context of introducing foreign DNA to a bacterial, yeast, plant, animal, or human cell. Cells capable of taking up foreign DNA are named competent cells. In other embodiments, transformation may be indicative for the insertion of new genetic material into animal and human cells, albeit the term “transfection” is more common for these cells.

Non-limiting examples of suitable transformation methods that can be applied to bacteria include heat-shock transformation and electroporation. In heat shock transformation, artificial competence is typically induced by making the cell permeable to DNA by subjecting them to non-physiological conditions. In such a typical transformation experiment, the cells are incubated in a solution containing divalent cations often in cold conditions, before the cells are exposed to a heat shock. It is theorized that exposure of the cells to divalent cations are responsible for a weakening of the cell surface structure, rendering it (more) permeable to DNA. The heat shock generates a thermal imbalance across the membrane, forcing entry of DNA through cell pores (i.e. adhesion zones or Bayer junctions) or through the damaged cell wall. An alternative method to induce transformation is by means of electroporation, which is hypothesized to create pores in the cellular membrane. In electroporation the bacterial cells are briefly exposed to an electric field of 10-20 kV/cm. After the shock, cellular membrane repair mechanisms remove the pores.

Unless explicitly stated otherwise, reference herein to any peptide, polypeptide, protein, or nucleic acid, or fragment thereof may generally also encompass modified forms of said peptide, polypeptide, protein, or nucleic acid, or fragment thereof, such as bearing post-expression modifications including the following non-limiting examples: phosphorylation, glycosylation, lipidation, methylation, cysteinylation, sulphonation, glutathionylation, acetylation, oxidation of methionine to methionine sulphoxide or methionine sulphone, combinations thereof.

The term “attenuated” as described herein can be used interchangeably with terms such as “weakened” and “diminished”. The wording “attenuated strain” is commonly used in the art and refers to weakened disease agents, i.e. attenuated pathogens. An attenuated bacterium is a weakened, less vigorous, less virulent bacterium when compared to the traditionally occurring counterpart. Multiple vaccines against different diseases are based on inclusion of an attenuated strain of a bacterium or virus that is still capable of inducing an immune response and creating immunity but not causing illness. An attenuated Mycoplasma bacterium according to embodiments of the invention is indicative for a genomically modified Mycoplasma bacterium wherein expression of genes whereof the gene product is responsible for a certain degree of virulence or toxicity have been modified in order to diminish or nullify the adverse effect of said gene on or in an infected subject.

Certain Mycoplasma (pneumoniae) genes such as Ca²⁺ dependent cytotoxic nuclease gene (MPN133) and ADP-ribosyltransferase CARDS gene (MPN372) have been described in the art to be involved in the toxicity of one or more Mycoplasma strains. Hence, the genetically modified Mycoplasma bacterium may comprise a functional modification in the MPN133 gene and/or operon. The genetically modified Mycoplasma bacterium may comprise a deletion, substitution, and/or insertion of one or more nucleotides in the MPN133 gene and/or operon. Alternatively, the genetically modified Mycoplasma bacterium may comprise a functional modification in the MPN372 gene and/or operon. The genetically modified Mycoplasma bacterium may comprise a deletion, substitution, and/or insertion of one or more nucleotides in the MPN372 gene and/or operon. However, the inventors have identified that when interfering with the function of both genes (i.e. when generating a single Mycoplasma strain comprising a mutation in at least MPN133 and MPN372), an unexpected synergistic effect can be observed while nevertheless maintaining viability in vivo of the resulting Mycoplasma bacterium as observed by the inventors.

Accordingly, a first aspect of the invention is directed to a genetically modified Mycoplasma bacterium comprising in its genome functional modifications in operons encoding Ca²⁺ dependent cytotoxic nuclease gene (MPN133) and ADP-ribosyltransferase CARDS gene (MPN372), wherein said functional modifications attenuate said Mycoplasma bacteria, i.e. that reduce the pathogenicity and/or immunogenicity of said Mycoplasma bacterium compared to a reference Mycoplasma bacterium, said reference Mycoplasma bacterium having an identical genomic sequence with the proviso that said reference Mycoplasma bacterium does not comprise a deletion, substitution, and/or insertion in one or more nucleotides of said operons. In an alternative yet complementing aspect, the invention is directed to a genetically modified Mycoplasma bacterium comprising in its genome a deletion, substitution, and/or insertion of one or more nucleotides in the operons encoding Ca²⁺ dependent cytotoxic nuclease gene (MPN133) and ADP-ribosyltransferase CARDS gene (MPN372) that reduce the pathogenicity and/or immunogenicity of said Mycoplasma bacterium compared to a reference Mycoplasma bacterium, said reference Mycoplasma bacterium having an identical genomic sequence with the proviso that said reference Mycoplasma bacterium does not comprise a deletion, substitution, and/or insertion in one or more nucleotides of said operons. In certain embodiments of the above aspects, the functional modifications reduce the expression level of MPN133 and/or MPN372 by 50%, preferably by 60%, preferably by 75%, preferably by 90%, preferably by 95%, preferably by 100%. In certain embodiments, the expression of MPN133and/or MPN372 is eliminated by the functional modification. Hence, a genetically modified Mycoplasma bacterium may be intended that does not transcribes and/or translates the MPN133 and/or MPN372 gene.

The terms “functional modification”, or “functional mutation” as used interchangeably herein indicates any kind of nucleotide mutation which functionally modifies the resulting sequence or its gene product and therefore functional modifications encompass any insertions, deletions, or substitutions of one or more nucleotides at one or more defined genomic positions. Alternatively worded, the functional modification be either be a deletion, insertion, and/or substitution, or may be caused by a deletion, insertion, and/or substitution. Therefore in the context of functional modifications, the wording “such as a deletion, insertion, and/or substitution” may be interchangeably used with “caused by a deletion, insertion, and/or substitution”. It is evident to a skilled person that the functional modification is caused by one or more deletions, insertions, and/or substitutions. Said defined genomic positions may be annotated by specification of the gene, or the operon comprising said gene. Alternatively, the genomic position of the functional modification may be indicated by any other suitable annotation means, such as but not limited to the IUBMB enzyme nomenclature. A skilled person is aware that any of these modifications or combinations of modifications is envisaged in this specification. Preferred functional modifications in a gene or operon are modifications that inactivate (i.e. inactivating mutations) and/or substitute a gene (i.e. gene substitutions, or substitutions of functional gene fragments), alternatively even substitute a complete operon. Further specific non-limiting examples of modifications are insertions or deletions that induce a frameshift in the reading frame of the gene or insertions or deletions that interrupt or remove a start codon or a Shine-Dalgarno sequence from a gene, or operably linked operon. Shine-Dalgarno sequences and methods for detecting them have been described in detail in the art (inter alia in Godbey, An introduction to biotechnology; the science, technology and medical applications, 2015). Frameshift mutations are generated for example by insertion or deletion of any integer number of nucleotides that is not a multiple of “3” or equal to “0”.

In certain embodiments where the functional modification such as a deletion, insertion, and/or substitution is in a gene or operon deemed non-essential for the survival and/or propagation of a Mycoplasma bacteria, a skilled person will appreciate that genetic ablation of said original gene or operon is envisaged in the context of the invention. In alternative embodiments where a functional modification such as a deletion, insertion, and/or substitution is introduced in an essential gene, said essential gene or operon may be partially or completely replaced with alternative genes or fragments of genes comprising an identical or similar function. Preferably the alternative genes or fragments of genes have an identical or similar function in or for the Mycoplasma bacterium but is/are less harmful for a host organism infected with the Mycoplasma bacterium. It is evident to a skilled person that numerous functional modifications such as deletions, insertions, and/or substitutions can be regarded as combinations of the above mentioned mutations and therefore fall within what is disclosed in this specification. A further envisaged modification is an inversion, or partial inversion of a sequence occurring in the genome of the Mycoplasma bacterium prior to the start of the process used to introduce the one or more functional modifications. In certain embodiments, the functional modification such as a deletion, insertion, and/or substitution may comprise insertion of a coding or non-coding barcode into one or more gene or operon beneficial for identifying, detecting, or authenticating the modified Mycoplasma bacterium, or aids in screening a population of Mycoplasma bacteria subjected to the process used to introduce the one or more functional mutations such as deletions, insertions, and/or substitutions to identify correctly modified Mycoplasma bacteria, or Mycoplasma bacteria that are suspected of being modified in a desired manner. In certain embodiments, the inserted or substituted nucleotides encode one or more selection markers.

“Selection markers”, “selectable markers”, or “phenotype markers” as used herein refer to genes that confer a trait suitable for artificial selection by a person skilled in the art. Commonly used selection markers are prokaryotic or eukaryotic antibiotic resistance genes not limited to ampicillin, chloramphenicol, tetracycline, kanamycin, blasticidin, neomycin, or puromycin. Alternatively, fluorescent markers are envisaged such as (enhanced)GFP, YFP, CFP, or mCherry. A skilled person appreciates that combinations of selection markers within a single modified Mycoplasma bacterium or even within a single genetic modification locus are envisaged.

In alternative embodiments the functional modification such as a deletion, insertion, and/or substitution alters the expression level of one or more genes. In further embodiments the expression level of one or more genes by said functional modification is downregulated by 1.5-fold, 2-fold, 2.5-fold, 5-fold, 10-fold, or more than 10-fold. In certain embodiments the functional modification such as a deletion, insertion, and/or substitution alters the expression level of the targeted gene or operon conditionally, i.e. when one or more criteria are met. For example, in an exemplary embodiment the functional modification such as a deletion, insertion, and/or substitution incorporates dependency of the expression level of one or more genes to compounds. In certain embodiments, the targeted gene or operon is provided with an inducible promoter, preferably a Tet-on or Tet-off promoter and transcription of the targeted gene or operon is subject to tetracycline-controlled transcriptional activation. Inducible promoters and expression systems have been described, as well as suitable tetracycline derivatives (inter alia in Krueger et al., Tetracycline derivatives: alternative effectors for Tet transregulators, BioTechniques, 2018). In certain embodiments wherein nucleotides are inserted or substituted, the newly introduced nucleotides are artificial nucleotides, commonly known in the art as Xeno nucleic acids (XNA) (Pinheiro and Holliger, Towards XNA biotechnology: new materials from synthetic genetic polymers, Trends in biotechnology, 2014). In certain embodiments, the functional modification such as a deletion, insertion, and/or substitution is present in a regulatory sequence, or comprises introduction or removal of a regulatory sequence.

“Operon” as used herein refers to a functional unit of DNA containing a cluster of genes in which all genes are controlled by a single promotor. It is evident to a skilled person that genes from an operon are co-transcribed. Transcribed genes from an operon are transcribed to a single mRNA strand and may be either translated together in the cytoplasm or spliced to generate monocistronic mRNAs that may be translated separately. A skilled person understands that multiple genetic elements in an operon are operably linked. The term “operably linked” refers to a multitude of genetic elements that are joined as part of the same nucleic acid molecule, suitably positioned and oriented for transcription to be initiated from the promoter. DNA operably linked to a promoter is under transcriptional initiation regulation of the promoter or in functional combination therewith.

The term “promoter” as defined herein is a region of DNA that initiates transcription of a particular gene and hence enables a gene to be transcribed. A promoter is recognized by RNA polymerase, which then initiates transcription. Thus, a promoter contains a DNA sequence that is either bound directly by, or is involved in the recruitment, of RNA polymerase. A promoter sequence can also include “enhancer regions”, which are one or more regions of DNA that can be bound with proteins (namely the transacting factors) to enhance transcription levels of genes in a gene-cluster. The enhancer, while typically at the 5′ end of a coding region, can also be separate from a promoter sequence, e.g., can be within an intronic region of a gene or 3′ to the coding region of the gene. Promoters may be located in close proximity of the start codon of genes, in preferred embodiments on the same strand and typically upstream (5′) of the gene. Promoters may vary in size, and are preferably from about 100 to 1000 nucleotides long. In certain embodiments, the promoter may be a constitutive promoter. A constitutive promoter is understood by a skilled person to be a promoter, whose expression is constant under the standard culturing conditions, i.e. a promoter which expresses a gene product at a constant expression level. In alternative embodiments, the promoter may be an inducible (conditional) promoter.

In certain embodiments, the genetically modified Mycoplasma bacterium as described herein comprises a functional modification such as a deletion, insertion, and/or substitution in at least one gene or operon comprising a gene selected from the group consisting of Table 1. Thus, the modified Mycoplasma bacterium as described herein may comprise a functional modification such as a deletion, insertion, and/or substitution in MPN133, MPN372 and at least one additional gene or operon, preferably at least two additional genes or operons, preferably at least four additional genes or operons, preferably at least five additional genes or operons, preferably at least six additional genes or operons selected from the group consisting of Table 1.

TABLE 1

Protein coding genes are termed MPN

| ORF | ID (Data Base) | ORF | ID (Data Base) | ORF | ID (Data Base) |
|---|---|---|---|---|---|
| MPN001 | id00013 | MPN006 | id00133 | MPN013 | id00227 |
| MPN002 | id00032 | MPN007 | id00143 | MPN014 | id00237 |
| MPN003 | id00049 | MPN008 | id00153 | MPN015 | id00254 |
| MPN004 | id00081 | MPN009 | id00174 | MPN016 | id00266 |
| MPN005 | id00114 | MPN010 | id00188 | MPN017 | id00275 |
| MPN020 | id00334 | MPN011 | id00207 | MPN018 | id00281 |
| MPN021 | id00375 | MPN012 | id00216 | MPN019 | id00306 |
| MPN022 | id00390 | MPN061 | id01193 | MPN106 | id02091 |
| MPN023 | id00406 | MPN062 | id01194 | MPN107 | id02124 |
| MPN024 | id00433 | MPN063 | id01207 | MPN108 | id02134 |
| MPN025 | id00443 | MPN064 | id01214 | MPN109 | id02159 |
| MPN026 | id00456 | MPN065 | id01230 | MPN110 | id02170 |
| MPN027 | id00493 | MPN066 | id01237 | MPN111 | id02214 |
| MPN028 | id00496 | MPN067 | id01282 | MPN112 | id02245 |
| MPN029 | id00513 | MPN068 | id01288 | MPN113 | id02252 |
| MPN030 | id00524 | MPN069 | id01291 | MPN114 | id02273 |
| MPN031 | id00534 | MPN070 | id01296 | MPN115 | id02306 |
| MPN032 | id00555 | MPN071 | id01315 | MPN116 | id02314 |
| MPN033 | id00563 | MPN072 | id01319 | MPN117 | id02319 |
| MPN034 | id00638 | MPN073 | id01336 | MPN118 | id02337 |
| MPN035 | id00642 | MPN074 | id01340 | MPN119 | id02338 |
| MPN036 | id00674 | MPN075 | id01341 | MPN120 | id02379 |
| MPN037 | id00711 | MPN076 | id01388 | MPN121 | id02386 |
| MPN037a | id00715 | MPN077 | id01412 | MPN122 | id02395 |
| MPN038 | id00726 | MPN078 | id01418 | MPN123 | id02422 |
| MPN039 | id00735 | MPN079 | id01451 | MPN124 | id02467 |
| MPN040 | id00754 | MPN080 | id01464 | MPN125 | id02468 |
| MPN041 | id00759 | MPN081 | id01511 | MPN126 | id02498 |
| MPN042 | id00767 | MPN082 | id01539 | MPN127 | id02514 |
| MPN043 | id00796 | MPN083 | id01566 | MPN128 | id02540 |
| MPN044 | id00818 | MPN084 | id01595 | MPN129 | id02562 |
| MPN044a | id00810 | MPN085 | id01627 | MPN130 | id02580 |
| MPN045 | id00819 | MPN086 | id01646 | MPN131 | id02598 |
| MPN046 | id00838 | MPN087 | id01652 | MPN132 | id02616 |
| MPN047 | id00859 | MPN088 | id01672 | MPN133 | id02636 |
| MPN047a& | id00867 | MPN089 | id01684 | MPN134 | id02653 |
| MPN048 | id00888 | MPN090 | id01695 | MPN135 | id02678 |
| MPN048a | id00876 | MPN091 | id01708 | MPN135a | id02687 |
| MPN049 | id00917 | MPN092 | id01722 | MPN136 | id02694 |
| MPN050 | id00971 | MPN093 | id01725 | MPN137 | id02728 |
| MPN051 | id00989 | MPN094 | id01748 | MPN138 | id02737 |
| MPN052 | id00993 | MPN095 | id01887 | MPN139 | id02747 |
| MPN053 | id01022 | MPN096 | id01899 | MPN140 | id02752 |
| MPN054 | id01044 | MPN097 | id01912 | MPN141 | id02773 |
| MPN055 | id01069 | MPN098 | id01938 | MPN142 | id02838 |
| MPN056 | id01096 | MPN099 | id01956 | MPN143 | id02888 |
| MPN057 | id01109 | MPN100 | id01977 | MPN144 | id02898 |
| MPN058 | id01119 | MPN101 | id01995 | MPN145 | id02925 |
| MPN059 | id01142 | MPN102 | id02026 | MPN146 | id02942 |
| MPN060 | id01157 | MPN103 | id02054 | MPN147 | id02956 |
| MPN060a | id01176 | MPN104 | id02053 | MPN148 | id02983 |
| MPN151 | id03032 | MPN104a | id02064 | MPN149 | id03001 |
| MPN152 | id03047 | MPN105 | id02077 | MPN150 | id03019 |
| MPN153 | id03087 | MPN194 | id03558 | MPN234 | id04292 |
| MPN153a | id03131 | MPN194a | id03569 | MPN235 | id04322 |
| MPN154 | id03137 | MPN195 | id03572 | MPN236 | id04337 |
| MPN155 | id03163 | MPN196 | id03592 | MPN237 | id04359 |
| MPN155a | id03160 | MPN197 | id03609 | MPN238 | id04386 |
| MPN156 | id03189 | MPN198 | id03635 | MPN239 | id04405 |
| MPN157 | id03196 | MPN199 | id03651 | MPN240 | id04421 |
| MPN158 | id03211 | MPN199a | id03672 | MPN241 | id04440 |
| MPN159 | id03223 | MPN200 | id03686 | MPN242 | id04452 |
| MPN160 | id03238 | MPN200a | id03681 | MPN243 | id04457 |
| MPN161 | id03259 | MPN201 | id03725 | MPN244 | id04492 |
| MPN162 | id03281 | MPN202 | id03743 | MPN245 | id04515 |
| MPN163 | id03301 | MPN203 | id03755 | MPN246 | id04510 |
| MPN164 | id03308 | MPN204 | id03769 | MPN247 | id04520 |
| MPN165 | id03313 | MPN205 | id03786 | MPN248 | id04531 |
| MPN166 | id03329 | MPN206 | id03808 | MPN249 | id04548 |
| MPN167 | id03336 | MPN207 | id03807 | MPN250 | id04560 |
| MPN168 | id03341 | MPN207a | id03803 | MPN251 | id04578 |
| MPN169 | id03357 | MPN208 | id03848 | MPN252 | id04587 |
| MPN170 | id03359 | MPN209 | id03861 | MPN253 | id04602 |
| MPN171 | id03367 | MPN210 | id03897 | MPN253a | id04611 |
| MPN172 | id03383 | MPN211 | id03931 | MPN254 | id04614 |
| MPN173 | id03391 | MPN212 | id03955 | MPN255 | id04625 |
| MPN174 | id03394 | MPN213 | id03965 | MPN256 | id04633 |
| MPN175 | id03398 | MPN214 | id04011 | MPN256a | id04643 |
| MPN176 | id03403 | MPN215 | id04015 | MPN257 | id04646 |
| MPN177 | id03409 | MPN216 | id04032 | MPN258 | id04660 |
| MPN177a | id03416 | MPN217 | id04052 | MPN259 | id04687 |
| MPN178 | id03417 | MPN218 | id04070 | MPN260 | id04710 |
| MPN179 | id03422 | MPN218a | id04108 | MPN261 | id04728 |
| MPN180 | id03427 | MPN219 | id04113 | MPN262 | id04765 |
| MPN181 | id03434 | MPN220 | id04118 | MPN263 | id04786 |
| MPN182 | id03441 | MPN221 | id04129 | MPN264 | id04794 |
| MPN183 | id03452 | MPN222 | id04135 | MPN265 | id04831 |
| MPN184 | id03458 | MPN223 | id04151 | MPN266 | id04833 |
| MPN185 | id03477 | MPN224 | id04163 | MPN267 | id04837 |
| MPN186 | id03490 | MPN225 | id04178 | MPN268 | id04855 |
| MPN187 | id03504 | MPN225a | id04184 | MPN269 | id04861 |
| MPN188 | id03507 | MPN226 | id04187 | MPN269a | id04882 |
| MPN189 | id03510 | MPN227 | id04194 | MPN270 | id04883 |
| MPN190 | id03518 | MPN228 | id04222 | MPN271 | id04891 |
| MPN191 | id03522 | MPN229 | id04235 | MPN271a | id04898 |
| MPN192 | id03539 | MPN230 | id04243 | MPN272 | id04936 |
| MPN193 | id03545 | MPN231 | id04249 | MPN273 | id04946 |
| MPN276 | id04978 | MPN232 | id04254 | MPN274 | id04956 |
| MPN277 | id04977 | MPN233 | id04274 | MPN275 | id04957 |
| MPN278 | id05004 | MPN319 | id05775 | MPN362 | id06562 |
| MPN278a | id05017 | MPN320 | id05805 | MPN363 | id06581 |
| MPN279 | id05046 | MPN321 | id05824 | MPN364 | id06586 |
| MPN280 | id05051 | MPN322 | id05833 | MPN365 | id06624 |
| MPN281 | id05076 | MPN323 | id05849 | MPN366 | id06642 |
| MPN282 | id05094 | MPN324 | id05859 | MPN367 | id06652 |
| MPN283 | id05125 | MPN325 | id05896 | MPN368 | id06679 |
| MPN284 | id05146 | MPN326 | id05902 | MPN369 | id06694 |
| MPN285 | id05191 | MPN327 | id05906 | MPN370 | id06706 |
| MPN286 | id05208 | MPN328 | id05915 | MPN371 | id06746 |
| MPN287 | id05241 | MPN329 | id05925 | MPN372 | id06748 |
| MPN288 | id05259 | MPN330 | id05934 | MPN373 | id06812 |
| MPN288a | id05267 | MPN331 | id05949 | MPN374 | id06826 |
| MPN289 | id05304 | MPN332 | id05968 | MPN375 | id06840 |
| MPN290 | id05312 | MPN333 | id05996 | MPN376 | id06896 |
| MPN291 | id05326 | MPN334 | id06034 | MPN376a | id06888 |
| MPN292 | id05335 | MPN335 | id06053 | MPN377 | id06907 |
| MPN293 | id05340 | MPN336 | id06098 | MPN378 | id06912 |
| MPN294 | id05341 | MPN337 | id06100 | MPN379 | id06950 |
| MPN295 | id05356 | MPN338 | id06125 | MPN380 | id06960 |
| MPN296 | id05366 | MPN339 | id06153 | MPN381 | id06976 |
| MPN297 | id05369 | MPN340 | id06165 | MPN382 | id06995 |
| MPN298 | id05376 | MPN341 | id06195 | MPN383 | id07023 |
| MPN299 | id05382 | MPN342 | id06224 | MPN384 | id07056 |
| MPN300 | id05392 | MPN343 | id06248 | MPN385 | id07061 |
| MPN301 | id05415 | MPN344 | id06266 | MPN386 | id07070 |
| MPN302 | id05426 | MPN345 | id06281 | MPN387 | id07090 |
| MPN303 | id05444 | MPN346 | id06303 | MPN388 | id07098 |
| MPN304 | id05492 | MPN347 | id06311 | MPN389 | id07110 |
| MPN305 | id05500 | MPN347a | id06328 | MPN390 | id07133 |
| MPN306 | id05514 | MPN348 | id06339 | MPN391 | id07155 |
| MPN307 | id05530 | MPN348a | id06334 | MPN391a | id07161 |
| MPN308 | id05544 | MPN349 | id06346 | MPN392 | id07174 |
| MPN309 | id05570 | MPN350 | id06366 | MPN393 | id07190 |
| MPN310 | id05589 | MPN351 | id06371 | MPN393a& | id07183 |
| MPN311 | id05659 | MPN352 | id06391 | MPN394 | id07211 |
| MPN312 | id05674 | MPN353 | id06420 | MPN395 | id07224 |
| MPN313 | id05687 | MPN354 | id06442 | MPN396 | id07270 |
| MPN314 | id05694 | MPN355 | id06452 | MPN397 | id07271 |
| MPN315 | id05699 | MPN356 | id06477 | MPN398 | id07313 |
| MPN316 | id05714 | MPN357 | id06502 | MPN399 | id07322 |
| MPN316a | id05730 | MPN357a | id06484 | MPN400 | id07348 |
| MPN317 | id05732 | MPN358 | id06503 | MPN401 | id07373 |
| MPN318 | id05748 | MPN359 | id06529 | MPN402 | id07379 |
| MPN405 | id07428 | MPN360 | id06542 | MPN403 | id07406 |
| MPN405a | id07438 | MPN361 | id06546 | MPN404 | id07411 |
| MPN406 | id07440 | MPN447 | id08329 | MPN489 | id09007 |
| MPN407 | id07490 | MPN448 | id08342 | MPN489a | id09017 |
| MPN408 | id07497 | MPN449 | id08362 | MPN490 | id09086 |
| MPN408a | id07495 | MPN450 | id08373 | MPN491 | id09089 |
| MPN409 | id07535 | MPN451 | id08386 | MPN491a& | id09102 |
| MPN410 | id07561 | MPN452 | id08410 | MPN492 | id09129 |
| MPN410a | id07566 | MPN453 | id08418 | MPN493 | id09138 |
| MPN411 | id07579 | MPN454 | id08427 | MPN494 | id09145 |
| MPN412 | id07593 | MPN455 | id08439 | MPN495 | id09150 |
| MPN413 | id07604 | MPN456 | id08490 | MPN496 | id09178 |
| MPN414 | id07614 | MPN457 | id08518 | MPN497 | id09195 |
| MPN415 | id07642 | MPN458 | id08528 | MPN498 | id09202 |
| MPN416 | id07662 | MPN459 | id08552 | MPN499 | id09212 |
| MPN417 | id07672 | MPN460 | id08590 | MPN500 | id09219 |
| MPN418 | id07702 | MPN461 | id08591 | MPN501 | id09244 |
| MPN419 | id07745 | MPN462 | id08601 | MPN502 | id09262 |
| MPN420 | id07756 | MPN463 | id08610 | MPN503 | id09287 |
| MPN421 | id07776 | MPN464 | id08611 | MPN504 | id09316 |
| MPN422 | id07797 | MPN464a | id08650 | MPN505 | id09333 |
| MPN423 | id07804 | MPN465 | id08661 | MPN506 | id09348 |
| MPN424 | id07807 | MPN466 | id08668 | MPN507 | id09390 |
| MPN425 | id07821 | MPN467 | id08672 | MPN508 | id09427 |
| MPN426 | id07858 | MPN468 | id08687 | MPN509 | id09463 |
| MPN427 | id07862 | MPN469 | id08722 | MPN509a | id09457 |
| MPN428 | id07899 | MPN470 | id08741 | MPN510 | id09482 |
| MPN429 | id07923 | MPN471 | id08743 | MPN511 | id09505 |
| MPN430 | id07935 | MPN472 | id08754 | MPN512 | id09521 |
| MPN431 | id07951 | MPN473 | id08766 | MPN513 | id09530 |
| MPN432 | id07968 | MPN474 | id08809 | MPN514 | id09550 |
| MPN433 | id07978 | MPN474a | id08811 | MPN515 | id09608 |
| MPN434 | id08000 | MPN475 | id08827 | MPN516 | id09681 |
| MPN435 | id08018 | MPN476 | id08830 | MPN517 | id09691 |
| MPN436 | id08071 | MPN477 | id08842 | MPN518 | id09718 |
| MPN436a | id08061 | MPN478 | id08852 | MPN519 | id09731 |
| MPN437 | id08106 | MPN479 | id08864 | MPN520 | id09735 |
| MPN438 | id08123 | MPN480 | id08906 | MPN521 | id09778 |
| MPN439 | id08134 | MPN481 | id08917 | MPN522 | id09788 |
| MPN440 | id08168 | MPN482 | id08921 | MPN523 | id09823 |
| MPN441 | id08177 | MPN482a | id08920 | MPN524 | id09841 |
| MPN442 | id08193 | MPN483 | id08922 | MPN525 | id09852 |
| MPN443 | id08212 | MPN484 | id08952 | MPN526 | id09868 |
| MPN444 | id08268 | MPN485 | id08978 | MPN527 | id09902 |
| MPN445 | id08265 | MPN486 | id08973 | MPN528 | id09903 |
| MPN446 | id08288 | MPN487 | id08982 | MPN528a | id09920 |
| MPN531 | id09961 | MPN488 | id09001 | MPN529 | id09928 |
| MPN532 | id09982 | MPN488a& | id09007 | MPN530 | id09930 |
| MPN533 | id09999 | MPN575 | id10650 | MPN619 | id11361 |
| MPN534 | id10005 | MPN576 | id10669 | MPN620 | id11389 |
| MPN534a | id10008 | MPN577 | id10688 | MPN621 | id11413 |
| MPN535 | id10013 | MPN578 | id10693 | MPN622 | id11416 |
| MPN536 | id10021 | MPN579 | id10709 | MPN623 | id11420 |
| MPN537 | id10060 | MPN580 | id10717 | MPN624 | id11447 |
| MPN538 | id10063 | MPN581 | id10734 | MPN625 | id11453 |
| MPN539 | id10071 | MPN582 | id10758 | MPN626 | id11466 |
| MPN539a | id10077 | MPN583 | id10780 | MPN627 | id11500 |
| MPN540 | id10078 | MPN583a | id10786 | MPN628 | id11524 |
| MPN541 | id10086 | MPN584 | id10795 | MPN629 | id11538 |
| MPN542 | id10088 | MPN585 | id10809 | MPN630 | id11562 |
| MPN543 | id10099 | MPN586 | id10823 | MPN631 | id11564 |
| MPN544 | id10113 | MPN587 | id10829 | MPN632 | id11578 |
| MPN545 | id10155 | MPN588 | id10855 | MPN633 | id11590 |
| MPN546 | id10168 | MPN589 | id10870 | MPN634 | id11603 |
| MPN547 | id10196 | MPN590 | id10876 | MPN634a | id11613 |
| MPN548 | id10210 | MPN591 | id10891 | MPN635 | id11625 |
| MPN549 | id10225 | MPN592 | id10918 | MPN636 | id11646 |
| MPN550 | id10242 | MPN592a | id10906 | MPN637 | id11657 |
| MPN551 | id10257 | MPN593 | id10923 | MPN638 | id11672 |
| MPN552 | id10275 | MPN594 | id10933 | MPN639 | id11692 |
| MPN553 | id10301 | MPN595 | id10936 | MPN640 | id11707 |
| MPN554 | id10307 | MPN596 | id10956 | MPN641 | id11718 |
| MPN555 | id10317 | MPN597 | id10962 | MPN642 | id11731 |
| MPN556 | id10339 | MPN598 | id10986 | MPN643 | id11737 |
| MPN557 | id10341 | MPN599 | id11002 | MPN644 | id11746 |
| MPN558 | id10362 | MPN600 | id11028 | MPN645 | id11756 |
| MPN559 | id10369 | MPN601 | id11038 | MPN646 | id11768 |
| MPN560 | id10398 | MPN602 | id11048 | MPN647 | id11780 |
| MPN561 | id10399 | MPN603 | id11054 | MPN648 | id11787 |
| MPN562 | id10423 | MPN604 | id11071 | MPN649 | id11806 |
| MPN563 | id10445 | MPN605 | id11075 | MPN650 | id11811 |
| MPN564 | id10468 | MPN606 | id11095 | MPN651 | id11826 |
| MPN565 | id10479 | MPN607 | id11097 | MPN652 | id11845 |
| MPN566 | id10487 | MPN608 | id11111 | MPN653 | id11853 |
| MPN567 | id10537 | MPN609 | id11125 | MPN654 | id11871 |
| MPN568 | id10552 | MPN610 | id11148 | MPN655 | id11877 |
| MPN569 | id10559 | MPN611 | id11171 | MPN655a | id11894 |
| MPN570 | id10564 | MPN612 | id11220 | MPN656 | id11900 |
| MPN571 | id10594 | MPN613 | id11239 | MPN657 | id11919 |
| MPN572 | id10620 | MPN614 | id11250 | MPN658 | id11925 |
| MPN573 | id10636 | MPN615 | id11268 | MPN659 | id11936 |
| MPN574 | id10642 | MPN616 | id11274 | MPN660 | id11941 |
| MPN663 | id11986 | MPN617 | id11283 | MPN661 | id11968 |
| MPN664 | id12002 | MPN618 | id11316 | MPN662 | id11977 |
| MPN665 | id12024 | MPN672 | id12145 | MPN681 | id12276 |
| MPN666 | id12025 | MPN672a | id12135 | MPN682 | id12279 |
| MPN667 | id12043 | MPN673 | id12151 | MPN683 | id12291 |
| MPN668 | id12065 | MPN674 | id12153 | MPN684 | id12369 |
| MPN669 | id12066 | MPN675 | id12181 | MPN685 | id12385 |
| MPN670 | id12096 | MPN676 | id12185 | MPN686 | id12405 |
| MPN671 | id12132 | MPN677 | id12223 | MPN687 | id12414 |
| MPN671a | id12104 | MPN678 | id12248 | MPN688 | id12426 |
| | | MPN679 | id12259 | | |
| | | MPN680 | id12272 | | |

The modified Mycoplasma bacterium as described herein may comprise a functional modification such as a deletion, insertion, and/or substitution in MPN133, MPN372 and at least one gene or operon comprising a gene classified in the art as gene involved in contributing to virulence, i.e. a virulence gene. In certain aspects the modified Mycoplasma bacterium as described herein comprises a functional modification such as a deletion, insertion, and/or substitution in MPN133, MPN372 and at least one gene, preferably at least two genes, preferably at least three genes, preferably at least four genes, preferably at least five genes, preferably at least six genes, that are known to be involved in Mycoplasma (pneumoniae) virulence. Mycoplasma virulence genes have been described in the art by Lluch-Senar and colleagues (Comparative “-omics” in Mycoplasma pneumoniae Clinical Isolates Reveals Key Virulence Factors, PLOS one, 2015). In further embodiments, the genetically modified Mycoplasma bacterium comprises a modified locus normally encoding Ca²⁺ dependent cytotoxic nuclease, wherein the modified locus comprises genetic elements that allow efficient insertion of exogenous genes.

In certain embodiments, the genetically modified Mycoplasma bacterium as described herein comprises a functional modification such as a deletion, insertion, and/or substitution in a gene or operon encoding a peroxide producing protein, preferably in a gene encoding glycerol 3-phosphate oxidase. In certain embodiments, the peroxide producing protein is glycerol 3-phosphate dehydrogenase (MPN051). In certain embodiments, the modified Mycoplasma bacterium comprises a functional modification such as a deletion, insertion, and/or substitution in MPN133, MPN372 and MPN051. In alternative embodiments, the modified Mycoplasma bacterium comprises a functional modification such as a deletion, insertion, and/or substitution in MPN051. In certain aspects wherein the modified Mycoplasma bacterium comprises a functional modification such as a deletion, insertion, and/or substitution in MPN051, said MPN051 gene or operon may be substituted, or at least partially substituted by a genetic element encoding a glyceraldehyde dehydrogenase capable of producing DHAP from G3P without producing peroxide, for example the gpsA enzyme or a functional fragment thereof. In preferred embodiments, the gpsA is the Mycoplasma penetrans gpsA protein, as characterized by Uniprot entry Q8EWH5. In further additional aspects, a genetically modified Mycoplasma bacterium is envisaged that comprises a functional modification such as a deletion, insertion, and/or substitution, leading to a truncation of the MPN051 gene product.

“Peroxide producing protein” as used herein refers to any protein that is involved in a direct or indirect manner in the production of peroxides in Mycoplasma. It has been disclosed in the art that for example M. pneumoniae adheres to and colonizes the surface of ciliated airway cells. During this process, the bacterium produces a large amount of hydrogen peroxide as product of the glycerol metabolism, which plays a crucial role in host cell cytotoxicity (Schmidl et al., A trigger enzyme in Mycoplasma pneumoniae: impact of the glycerophosphodiesterase GlpQ on virulence and gene expression, PLOS Pathogens, 2011). A skilled person appreciates that peroxide production is an unwanted characteristic for Mycoplasma that is attenuated, and/or has a reduced toxicity when introduced in a host organism. The genetically modified Mycoplasma bacterium may therefore comprise a functional modification in a nuclease gene or operon encoding a nuclease gene and a gene or operon encoding a toxin and a gene or operon encoding a protein involved in peroxide production.

In certain embodiments, the genetically modified Mycoplasma as described herein comprises a functional modification such as a deletion, insertion, and/or substitution in a gene or operon encoding a second nuclease gene. In certain embodiments, the functional modification such as a deletion, insertion, and/or substitution is in a surface nuclease gene. In further embodiments, the further functional modification such as a deletion, insertion, and/or substitution is in membrane nuclease A (MPN491). Therefore, the genetically modified Mycoplasma bacterium may comprise a function modification such as a deletion, insertion, and/or substitution in MPN133, MPN372, and MPN491.

The term “nucleases” as used herein, also known as “nucleodepolymerases” and “polynucleotidases” are a group of enzymes that effectuate cleavage of phosphodiester bonds between the nucleotides of nucleic acids (i.e. molecular scissors). Both nucleases that are capable of inducing single stranded breaks and double stranded breaks have been described in the art. It is known that there is a large diversity in terms of structure and function among different nucleases. Nucleases can be either endonucleases that lead to the generation of oligonucleotides as a consequence of their activity, or exonucleases that have single nucleotides as cleavage products.

In certain embodiments, the genetically modified Mycoplasma as described herein comprises a functional modification such as a deletion, insertion, and/or substitution in one or more genes or operons encoding a cytoadherence protein. In preferred embodiments, the gene or operon encoding a cytoadherence gene is selected from the group consisting of MPN141, MPN 142, MPN453, MPN447, MPN310, MPN452, MPN309. In certain embodiments, the modified Mycoplasma bacteria comprises a functional modification such as a deletion, insertion, and/or substitution in MPN133 and MPN372 genes or operons and in one or more genes or operons selected from the group consisting of MPN141, MPN 142, MPN453, MPN447, MPN310, MPN452, MPN309. In certain embodiments, the modified Mycoplasma bacterium has a functional modification such as a deletion, insertion, and/or substitution in MPN133, MPN372, and MPN142. In certain aspects, a modified Mycoplasma bacterium may comprise a functional modification such as a deletion, insertion, and/or substitution in MPN133, MPN372, MPN051, and MPN142. In certain aspects a genetically modified Mycoplasma bacteria may comprise a functional modification such as a deletion, insertion, and/or substitution in MPN453. In further embodiments, the genetically modified Mycoplasma bacteria may comprise a functional modification such as a deletion, insertion, and/or substitution in MPN133, MPN372, MPN453, and optionally in MPN051. In further aspects, the genetically modified Mycoplasma bacteria may comprise a functional modification such as a deletion, insertion, and/or substitution in MPN133, MPN453, and MPN453.

The term “cytoadherence” which is used in the context of cytoadherent proteins in the context of this specification is indicative for any protein that is responsible for, or aids in the adhesion of Mycoplasma to tissue of the host organism. For example, when Mycoplasma pneumoniae is envisaged, a cytoadherence protein within the context as defined herein is any protein that is responsible for, or aids in adhesion of M. pneumoniae onto the respiratory epithelia of a host organism, preferably to sialoglycoproteins and/or sulphated glycolipids of epithelial cells. Preferably, the adhesion is effected by a specialized organelle comprising adhesins and accessory proteins as described in the art (Shimizu, Inflammation-inducing factors of Mycoplasma pneumoniae, Frontiers in microbiology, 2016). In certain embodiments, the genetically modified Mycoplasma bacteria comprising a functional modification in a gene or operon encoding a protein involved in cytoadherence are characterized by improved growth in a bio production vessel, such as the non-limiting example of a fermenter. In further embodiments, the genetically modified Mycoplasma bacteria are characterized by an ability to grow in suspension.

In certain embodiments, the genetically modified Mycoplasma bacterium as described herein comprises a functional modification such as a deletion, insertion, and/or substitution in one or more genes encoding an immunogenic protein that is capable of eliciting an immune response in a host organism. In further embodiments, the immunogenic protein is the conserved hypothetical protein MPN400. Therefore, the modified Mycoplasma bacterium may comprise a functional modification such as a deletion, insertion, and/or substitution in MPN133, MPN372, and MPN400. Alternatively, the modified Mycoplasma bacterium may comprise a functional modification in MPN133, MPN372, MPN400, and MPN051. Alternatively, the genetically modified Mycoplasma bacterium may comprise a functional modification such as a deletion, insertion, and/or substitution in MPN133, MPN372, MPN294, and MPN400. Also envisaged is a genetically modified Mycoplasma bacterium comprising a functional modification such as a deletion, insertion, and/or substitution in MPN372 and MPN294.

An immune response in the context of the current specification is a reaction which occurs within an organism for the purpose of counteracting a foreign invading organism by the host organism subject of exposure to said foreign organism. In certain prior art documents, an immune response is commonly described as repelling or inhibiting a foreign organism from proliferating or surviving in a host organism. Alternatively, the purpose of an immune response is known in the art to be the safeguarding of the host organism from the invading organism. A skilled person understands that in general, an immune response leads to an improved health state of the infected organism. However, in certain instances the improved health state of the infected organism is characterized by a prior temporary decrease in health state of the infected organism. Both genes encoding for gene products that may lead to an innate or an adaptive immune response are envisaged by the inventors. It is evident to a skilled person that “an immune response” can be a result of a reaction of the host organism against one or multiple distinct immunogenic factors. Exemplary examples of immunogenic proteins are proteins expressed on the surface of pathogens, proteins released by pathogens, or metabolites of foreign proteins.

In certain embodiments, the genetically modified Mycoplasma bacterium as described herein comprises a functional modification such as a deletion, insertion, and/or substitution in a gene or operon encoding a protein that contributes to, or is responsible for, the development of an autoimmune disease in a host organism. In further embodiments, the autoimmune disease is an autoimmune neuropathy. In yet a further embodiment, the autoimmune disease is the Guillain-Barre syndrome which presumably is caused by monogalactosylceramide. However, research of the inventors in fact evidences that patient antibodies in fact mainly recognize dihexo-ceramides and not monogalactosylceramide. This finding is important when aiming to develop Mycoplasma bacteria having a minimal chance to give rise to Guillain-Barre syndrome in the patient, or do not even lead to Guillain-Barre in any patient. Therefore in certain aspects, a genetically modified Mycoplasma bacterium may comprise a functional modification such as a deletion, insertion, and/or substitution in MPN257, which encodes UDP-glucose 4-epimerase. In other aspects, a genetically modified Mycoplasma bacterium may comprise a functional modification such as a deletion, insertion, and/or substitution in MPN483, which encodes a processive UDP-glycosyltransferase. In further aspects, a genetically modified Mycoplasma as described herein may comprise a functional modification such as a deletion, insertion, and/or substitution in MPN257 and MPN483. In further aspects, a genetically modified Mycoplasma may comprise a functional modification such as a deletion, insertion, and/or substitution in MPN133 and/or MPN 372 in addition to a functional modification such as a deletion, insertion, and/or substitution in MPN257 and/or MPN483, while optionally also comprising a functional modification such as a deletion, insertion, and/or substitution in MPN051. In certain embodiments, the genetically modified Mycoplasma bacterium as described herein comprises a functionally modification in MPN257 and/or MPN483, while also comprising a functional modification such as a deletion, insertion, and/or substitution in at least one gene, preferably at least two genes, preferably at least four genes, preferably at least five genes, preferably at least six genes, preferably at least three genes, selected from the group consisting of Table 1. In certain embodiments, the genetically modified Mycoplasma bacterium as described herein comprises a functionally modification such as a deletion, insertion, and/or substitution in MPN257 and/or MPN483, while also comprising a functional modification such as a deletion, insertion, and/or substitution in MPN051 and at least one gene, preferably at least two genes, preferably at least four genes, preferably at least five genes, preferably at least six genes, preferably at least three genes, selected from the group consisting of Table 1.

“Guillain-Barre syndrome” as indicated herein refers to an autoimmune disease affecting the peripheral nervous system. It has been characterized in the art as a rapid-onset polyradiculoneuropathy typically accompanied by sensory symptoms and weakness which often leads to quadriparesis (Donofrio, Guillain-Barre syndrome, Continuum: lifelong learning in neurology, 2017). On neurological examination, characteristic features are the reduced strength of muscles and reduced or absent tendon reflexes (hypo- or areflexia, respectively). However, some patients display normal reflexes in affected limbs before developing areflexia, and some may have exaggerated reflexes. A portion of the patients further develop weakness of the breathing muscles leading to respiratory failure which may be detrimental to patients characterized by additional medical conditions including but not limited to pneumonia. Additionally, a portion of the patients show perturbed autonomic nervous system. Although preceding respiratory tract infections with M. pneumoniae have been reported in some cases, the role of M. pneumoniae in the pathogenesis of GBS remains unclear. M. pneumoniae infection is associated with GBS, more frequently in children than adults, and elicits anti-GalactoCerebroside (GalC) antibodies, of which specifically anti- GalC IgG may contribute to the pathogenesis of GBS. Up to the present research performed by the inventors, it was assumed that these antibodies mainly recognize monogalactosylceramide. However, the inventors have obtained compelling evidence that the antibodies in fact recognize dihexose-ceramides. Antibodies against GalC concomitant to evidence of M. pneumoniae infection also have been associated to encephalitis and other nervous disorders. Hence, it is a further object of the invention to develop and characterize Mycoplasma (pneumoniae) strains that have a reduced risk on provoking Guillain-Barre syndrome in a subject after administration of the bacterium.

To an expert in the field, deletion of MPN257 will prevent the formation of Galatoceramides and galatodiacylglycerol, while deletion of MPN483 will result in prevention of formation of monohexose-ceramides and monohexoseodiacylglycerol if the two other glycosil transferases (MOPN028 and MPN075) cannot catalyze those reactions, and of dihexose-ceramide and dihexosediacylglycerol if they can (MPn483 is a processive glycosyltransferase). Thus if dihexoseceramieds containg galatose are responsible of causing autoimmunity, deletion of MPN257 and//or MPN483 removes the possibility of M. pneumoniae to trigger autoimmunity and develop Guillain-Barre Syndrome.

However, single or double knock-out of MPN483 in M. pneumoniae strains impacts the growth of said M. pneumoniae adversely. Without wishing to be bound by theory, this observation may stem from an accumulation of toxic ceramides in M. pneumoniae. Hence, in certain embodiments wherein the genetically modified Mycoplasma bacterium comprises a functional modification such as a deletion, insertion, and/or substitution in either MPN483, or both MPN483 and MPN257, said Mycoplasma bacterium may contain an exogenous gene sequence or operon that encodes a gene product from one or more different Mycoplasma species that are able to partially or completely negate the reduction in growth rates of the above mentioned MPN483 and/or MPN257 knock outs while preventing formation of galatosylceramides or galatosyldiacylglycerol. By means of example, suitable exogenous genes capable of (partially) reverting the consequences of MPN483 inactivation or deletion are glycosyltransferases from orthologue Mycoplasma species, such as but not limited to M. genitalium MG_517 (orthologue of MPN4893 that uses preferentially UDP-glucose to UDP-galatose), M. agalactiae MAGA_RS00300 (orthologue of MPN483 in Mycoplasma agalactiae that does not contain the orthologue of MPN257 and therefore does not produce UDP-galactose), B. subtilis utgP (processive glycosyltransferase that only uses UDP-glucose), or any combination thereof. Unexpectedly, the inventors have demonstrated that MG_517, MAGA RS00300, and to a lesser extent utgP are able to mitigate the suboptimal growth rate of M. pneumoniae strains lacking a functional MPN483 gene. The reduced growth rate is more pronounced in mutant strains lacking functional MPN483 when compared to mutants lacking functional MPN257, and hence a preferred genetically modified Mycoplasma bacterium is a M. pneumoniae bacterium that does not comprise neither a functional MPN483 gene nor MPN257 gene, wherein the enzymatic activity of MPN483 is partially or completely replaced by insertion of MG_517, MAGA RS00300, and/or utgP.

In certain embodiments, the genetically modified Mycoplasma bacterium as described herein comprises a functional modification such as a deletion, insertion, and/or substitution in a gene or operon encoding a protein that inhibits growth of said bacterium in a bioreactor. Thus, said functional modification such as a deletion, insertion, and/or substitution may ameliorate growth of the genetically modified Mycoplasma in a bioreactor. In further embodiments, the gene or operon encodes the chaperone protein YajL (MPN294). In certain embodiments, the modified Mycoplasma bacterium comprises a functional modification such as a deletion, insertion, and/or substitution in MPN372 and MPN 294. In further embodiments, the modified Mycoplasma bacterium comprises a functional modification such as a deletion, insertion, and/or substitution in MPN133, MPN372 and MPN294, and optionally comprises a functional modification such as a deletion, insertion, and/or substitution in MPN051. In certain embodiments, the growth rate of said modified bacterium in a bioreactor is elevated by introduction of the one or more functional modifications such as deletions, insertions, and/or substitutions. In certain embodiments, the one or more functional modifications such as deletions, insertions, and/or substitutions change the Mycoplasma bacterium growth manner from an adherent growth pattern to a suspension growth pattern. The modified Mycoplasma bacterium may comprise one or more functional modifications such as deletions, insertions, and/or substitutions that alter the morphology of the Mycoplasma bacterium. In certain embodiments, the genetically modified Mycoplasma bacterium has a doubling time that is reduced (i.e. shortened) by at least 20%, preferably by at least 25%, preferably by at least 40%, preferably by at least 50%, preferably by at least 60%, preferably by at least 75% when compared to a Mycoplasma bacterium not comprising said combination of functional modified genes. The genetically modified Mycoplasma bacterium may in certain aspects comprise one or more functional modifications such as deletions, insertions, and/or substitutions that reduce the doubling time of the modified bacteria as described above only when a certain nutrient is present in a culture medium or propagation location. A skilled person is aware that any comparative analysis between different strains should be performed in identical culture conditions. The genetically modified Mycoplasma bacteria may comprise a functional modification such as a deletion, insertion, and/or substitution in a genetic element such as but not limited to MPN294 that allows for inducible regulation of gene expression.

The terms “doubling time” and “generation time” are standard terms in the art and indicate the time taken by a bacterium to double in number during a specified time period known as the generation time. The generation time varies between different organisms. Non-limiting examples of physical factors include pH temperature, pressure, and moisture content. Nutritional factors include for example the amount of carbon, nitrogen, sulfur, and phosphor. When doubling time is assessed of the genetically modified bacterium in a host organism, it is evident that equivalent host organisms are required to assess doubling time. A skilled person is aware that numerous parameters can impact bacterial doubling time in a host organism, including but not limited to the immune status of the host organism, age, body weight, food intake, gender, etc. It has been described in detail in the art that the growth of bacteria can be characterized by different phases, of which the log phase (alternatively logarithmic phase or exponential phase) indicates the growth phase wherein bacteria are doubling. If growth is not limited, the doubling will continue at a constant rate so both the number of cells and the rate of population increase duplicates within a given time period. The slope of this line indicates the growth rate of the organism.

In certain embodiments, the genetically modified Mycoplasma bacterium as described herein comprises a functional modification such as a deletion, insertion, and/or substitution in one or more genes encoding a lipoprotein. In further embodiments, the gene or operon encoding a lipoprotein is selected from the group consisting of: MPN141, MPN142, MPN152, MPN162, MPN199, MPN200, MPN224, MPN233, MPN271, MPN284, MPN288, MPN293, MPN333, MPN372, MPN415, MPN447, MPN592, MPN597, MPN602, MPN611, MPN011, MPN052, MPN054, MPN058, MPN083, MPN084, MPN097, MPN098, MPN363, MPN369, MPN408, MPN411, MPN436, MPN439, MPN442, MPN444, MPN456, MPN467, MPN489, MPN506, MPN523, MPN582, MPN585, MPN586, MPN587, MPN588, MPN590, MPN591, MPN592, MPN639, MPN640, MPN641, MPN642, MPN643, MPN644, MPN645, MPN646, MPN647, MPN648, MPN649, MPN650, MPN654. In certain embodiments, the modified Mycoplasma bacterium comprises a functional modification such as a deletion, insertion, and/or substitution in MPN133, MPN372, and in a lipoprotein encoding gene or operon selected from the group consisting of: MPN141, MPN142, MPN152, MPN162, MPN199, MPN200, MPN224, MPN233, MPN271, MPN284, MPN288, MPN293, MPN333, MPN372, MPN415, MPN447, MPN592, MPN597, MPN602, MPN611, MPN011, MPN052, MPN054, MPN058, MPN083, MPN084, MPN097, MPN098, MPN363, MPN369, MPN408, MPN411, MPN436, MPN439, MPN442, MPN444, MPN456, MPN467, MPN489, MPN506, MPN523, MPN582, MPN585, MPN586, MPN587, MPN588, MPN590, MPN591, MPN592, MPN639, MPN640, MPN641, MPN642, MPN643, MPN644, MPN645, MPN646, MPN647, MPN648, MPN649, MPN650, MPN654, and optionally further comprising a functional modification such as a deletion, insertion, and/or substitution in MPN051. In further embodiments, the genetically modified Mycoplasma bacterium as described herein comprises a functional modification such as a deletion, insertion, and/or substitution in the prolipoprotein diacylglyceryl transferase gene (MPN224) or operon and/or the lipoprotein signal peptides gene MPN293 or operon. The genetically modified Mycoplasma may comprise a functional modification such as a deletion, insertion, and/or substitution in MPN133 and/or MPN372, in addition to a functional modification such as a deletion, insertion, and/or substitution MPN224 and/or MPN293, and optionally a functional modification such as a deletion, insertion, and/or substitution in MPN051.

In certain embodiments, the genetically modified Mycoplasma bacterium as described herein comprises a functional modification such as a deletion, insertion, and/or substitution in a gene or operon encoding an oncogenic protein. In further embodiments, the functional modification such as a deletion, insertion, and/or substitution is comprised in a gene or operon encoding the high affinity transport system protein p37 (MPN415). In certain embodiments, the modified Mycoplasma bacterium comprises a functional modification such as a deletion, insertion, and/or substitution in MPN133, MPN372 and MPN415, and optionally comprises a functional modification such as a deletion, insertion, and/or substitution in MPN051. In an aspect, a genetically modified Mycoplasma bacterium as envisaged herein is a Mycoplasma bacterium comprising a functional modification such as a deletion, insertion, and/or substitution in MPN415.

The term “oncogenic protein” as used herein refers to any protein that is present in a Mycoplasma bacteria that, when introduced or expressed in a host organism, preferably a mammal, significantly increases the risk of said organism to develop cancer, characterized by an increased and/or uncontrolled division of cells of one or more cell types present in the host organism.

In certain embodiments, the genetically modified Mycoplasma bacterium as described herein comprises a functional modification such as a deletion, insertion, and/or substitution in a gene or operon encoding an RNA polymerase factor. In further embodiments, the gene or operon encodes probable RNA polymerase sigma-D factor (MPN626). In certain embodiments, the modified Mycoplasma bacterium comprises a functional modification such as a deletion, insertion, and/or substitution in MPN133, MPN372, MPN626, and optionally in MPN051. The genetically modified Mycoplasma bacterium may comprise a functional modification such as a deletion, insertion, and/or substitution in a gene or operon encoding a protein involved in DNA replication. In an aspect, a genetically modified Mycoplasma bacterium as envisaged herein is a modified Mycoplasma bacterium comprising a functional modification such as a deletion, insertion, and/or substitution in MPN626. In a further aspect, a genetically modified Mycoplasma bacterium as described herein comprises a modified locus normally encoding the RNA polymerase sigma-D factor, wherein the modified locus comprises genetic elements that allow efficient insertion of exogenous genes.

In certain embodiments, the genetically modified Mycoplasma bacterium as described herein comprises a functional modification such as a deletion, insertion, and/or substitution in a gene or operon encoding a secreted Mycoplasma gene product, preferably a secreted Mycoplasma protein. In further embodiments, the functional modification such as a deletion, insertion, and/or substitution is in a gene or operon encoding a gene selected from the group consisting of MPN400, MPN036, MPN592, MPN509, MPN647, MPN084, MPN625, MPN213, MPN489, MPN142, MPN444, MPN642, MPN398, MPN491, MPN083 and MPN141. In further embodiments, the modified Mycoplasma bacterium comprises a functional modification such as a deletion, insertion, and/or substitution in MPN133and MPN372 and additionally one or more genes selected from the group consisting of MPN400, MPN036, MPN592, MPN509, MPN647, MPN084, MPN625, MPN213, MPN489, MPN142, MPN444, MPN642, MPN398, MPN491, MPN083, MPN141 and optionally comprises a functional modification such as a deletion, insertion, and/or substitution in MPN051. The genetically modified Mycoplasma bacteria may comprise a functional modification such as a deletion, insertion, and/or substitution in MPN133, MPN372, MPN400. Further, the genetically modified Mycoplasma bacteria may comprise a functional modification such as a deletion, insertion, and/or substitution in MPN133, MPN372, MPN400, and in MPN051.

“Secreted gene products” as used herein refers to any gene product that is secreted by a cell, in the context of this specification a bacterial cell, preferably a Mycoplasma bacterium. It is evident that a “secreted gene product” may in addition to full length gene products such as proteins also refer to (partially) degraded gene products such as metabolites.

In certain embodiments, the degree of attenuation of the genetically modified Mycoplasma as described herein is indicated by a reduction in toxicity of said Mycoplasma bacterium when introduced in a host organism when compared to a reference Mycoplasma bacterium, wherein said reference Mycoplasma bacterium is a naturally occurring (wild type) Mycoplasma bacterium not comprising a genetic functional modification such as a deletion, insertion, and/or substitution. A preferred reference Mycoplasma bacterium is M. pneumoniae M129-B7. In certain embodiments, the toxicity is reduced by 10%, preferably by 25%, preferably by 50%, preferably by 60%, preferably by 70%, preferably by 85%, preferably by 90%, preferably by 95%, when introduced in a host organism. In certain embodiments, the reduction of toxicity is indicative for the reduction of toxicity when the modified Mycoplasma is introduced to the respiratory system, preferably the lungs, of said host organism, when compared to the toxicity of a wild type Mycoplasma bacterium.

In certain aspects, a genetically modified Mycoplasma bacterium is envisaged comprising a deletion of one or more genomic regions comprising one or more genes disclosed herein. By means of guidance and not limitation, a genetically modified Mycoplasma bacterium may comprise a deletion from MPN490 up to and optionally including MPN505. Alternatively, as illustrative example a genetically modified Mycoplasma bacterium may comprise a deletion from MPN490 up to and optionally including MPN506.

In certain embodiments, the genetically modified Mycoplasma bacterium as described herein is a Mycoplasma pneumoniae bacterium. Transformation protocols specific for M. pneumoniae are disclosed in the art (Krishnakumar et al., Targeted chromosomal knockouts in Mycoplasma pneumoniae, Applied and environmental microbiology, 2010). In certain embodiments, the Mycoplasma pneumoniae bacterium comprises one or more artificial genetic element that facilitates genomic insertion of nucleotide sequences that encode for genes capable of producing exogenous gene products. In further embodiments, the genetically modified Mycoplasma bacterium is a Mycoplasma bacterium isolated from a subject diagnosed with, or suspected to have pneumonia. In further embodiments, the Mycoplasma bacterium is isolated from the respiratory system, for example the lungs or the trachea, of said subject.

In certain embodiments, the genetically modified Mycoplasma bacterium as described herein comprises a nucleotide sequence encoding an exogenous gene product or a functional fragment thereof. In preferred embodiments, said nucleotide sequence is comprised in the genomic sequence of said Mycoplasma bacterium. The exogenous gene product can be codon-optimized for expression in Mycoplasma. The exogenous gene product can be controlled by a naturally occurring Mycoplasma promoter. Alternatively, the nucleotide sequences encoding an exogenous gene product or functional fragment thereof are part of a non-genomically integrated expression vector. Non-limiting examples of expression vectors described in the art include plasmids, optionally non-replicative plasmids, phagemids, bacteriophages, bacteriophage-derived vectors, artificial chromosomes, minicircles, lentiviral vectors, retroviral vectors, adenoviral or adeno-associated viral vectors, piggyback vectors, or tol2 vectors. Furthermore, it is evident to a skilled person that plasmid DNA, or recombinant DNA is commonly referred to in the art as copy DNA, complement DNA, or even referred to by the abbreviation “cDNA”. Hence, the nucleotide arrangements may be part of a bicistronic or multicistronic expression construct. Optionally, the exogenous gene product is an artificially designed gene product. In certain embodiments, the exogenous gene product is a protein. In certain embodiments, the exogenous gene product is and/or replaces an original glycosyltransferase or an original UDP-glucose epimerase or a combination of a glycosyltransferase and a UDP-glucose epimerase, for instance the MPN483 gene may be replaced by M. genitalium MG_517, M. agalactiae MAGA_RS00300, and/or B. subtilis UgtP.

“Gene product” as used herein is indicative for any molecule directly derived from a gene or functional fragment of a gene. A skilled person is aware that the term “gene product” may also be indicative for the product derived from a non-naturally occurring operon comprised in a Mycoplasma bacterium, as indicated by the term “heterologous gene product” or “exogenous gene product”. The term may therefore cover any protein of biotechnological interest.

In certain embodiments, the gene product comprises one or more regulatory sequences. “Control sequences” or “regulatory sequences” as used interchangeably herein refer to any nucleotide sequence which capable of increasing or decreasing the expression of specific genes. This regulation may be imposed by either influencing transcription rates, translation rates, or by modification of the stability of the sequence. In further embodiments, the polynucleotide sequence comprises regulatory elements such as but not limited to the following: enhancers, selection markers, origins of replication, linker sequences, polyA sequences, terminator sequence, and degradation sequences. Non-limiting examples of particularly suited regulatory sequences are secretory signals that may be naturally occurring Mycoplasma secretion signals or non-naturally occurring Mycoplasma sequences, the latter being described in detail in International patent application WO2016/135281.

In certain aspects, a genetically modified Mycoplasma bacterium as described herein may comprise an insertion of one or more genes encoding proteins, said proteins selected from the group comprising: Mycoplasma pulmonis Vsa (such as Vsa with Uniprot database entry Q50279), Mycoplasma penetrans GpsA (Uniprot database entry Q8EWH5), and Mycoplasma hypopneumoniae P97 (Uniprot database entry Q49542).

In further embodiments, said exogenous gene product or functional fragment thereof is a protein. In preferred embodiments, the exogenous gene product is a therapeutic protein, a protein involved in specific attachment to a host protein, an enzyme, immunogenic protein, or DNA-binding protein. In further embodiments, the said therapeutic or immunogenic protein is expressed on the surface of said Mycoplasma bacterium and/or is secreted by said Mycoplasma bacterium. The heterologous nucleotide-encoded gene product may be any protein or peptide that has an advantageous effect for the Mycoplasma bacterium, infected host, or environment. In certain embodiments, the exogenous gene product is a fusion protein. In certain embodiments wherein the exogenous gene product or functional fragment thereof is a fusion protein, said protein comprises a protease site between two or more functional fragments of said protein. It is evident for a person skilled in the art that concatenation of multiple nucleotide sequences encoding a certain protein and afterwards separating the multiple copies of said protein by a protease is a suitable yet not limiting manner to increase the amount of protein that is produced. In certain embodiments, the exogenous gene product or functional fragment thereof binds to a protein expressed by the host organism. In further embodiments, the exogenous gene product or functional fragment thereof binds to a protein expressed by the host organism by a specific tissue and/or cell type. In yet further embodiments the exogenous gene product or functional fragment thereof binds to a protein expressed by the host organism on the membrane of a specific cell type. In alternative embodiments the exogenous gene product or functional fragment thereof binds to a protein expressed by a distinct pathogenic or non-pathogenic organism present in the host organism. In further embodiments the exogenous gene product or functional fragment thereof binds to a protein expressed by a distinct pathogenic or non-pathogenic organism present in the respiratory tract of the host organism. In yet further embodiments the exogenous gene product or functional fragment thereof is cytotoxic for said distinct pathogenic or non-pathogenic organism present in the respiratory tract of the host organism. In further embodiments the exogenous gene product or functional fragment thereof inhibits replication of said distinct pathogenic or non-pathogenic organism present in the host organism. In certain embodiments the exogenous gene product is a DNA-binding protein and specifically binds to a DNA sequence not occurring in the host organism. In further embodiments the exogenous gene product or functional fragment thereof is a designer nuclease.

The term “therapeutic protein” or “therapeutic peptide” is considered clear to a person skilled in the art and the skilled person understands that a wide range of therapeutic proteins have been described in the art. Therapeutic proteins can be stratified into five large groups: (a) replacing a protein that is deficient or abnormal; (b) augmenting an existing pathway; (c) providing a novel function or activity; (d) interfering with a molecule or organism; and (e) delivering other compounds or proteins, such as a radionuclide, cytotoxic drug, or effector proteins. Alternatively, therapeutic proteins may also be grouped based on their molecular types that include antibody-based drugs, Fc fusion proteins, anticoagulants, blood factors, bone morphogenetic proteins, engineered protein scaffolds, enzymes, growth factors, hormones, interferons, interleukins, and thrombolytics. Therapeutic proteins and therapeutic peptides can also be classified based on their molecular mechanism of activity as (a) binding non-covalently to target such as monoclonal antibodies; (b) affecting covalent bonds such as enzymes; and (c) exerting activity without specific interactions, e.g. serum albumin. The above mentioned classifications and the contribution of each of the groups to the state of the art have been described in scientific literature (Dimitrov, Therapeutic proteins, Methods in Molecular Biology, 2012). Non-limiting examples of classes of therapeutic proteins include cytokines, antibodies, nanobodies, (soluble) receptors, antibody-like protein scaffolds, and functional fragments hereof. Hence, in further embodiments where the gene product is a protein the gene product may further comprise a nucleotide-encoded peptide or protein tag sequence. Non-limiting examples of commonly used peptide tag sequences are the AviTag, C-tag, calmodulin-tag, polyglutamate tag, E-tag, Flag-tag, HA-tag, His-tag, Myc-tag, NE-tag, Rho1D4-tag, S-tag, SBP-tag, Softag 1, Softag 3, Spot-tag, Strep-tag, TC tag, Ty tag, V5 tag, VSV-tag, Xpress tag, isopeptag, SpyTag, SnoopTag, DogTag, and the SdyTag. In further embodiments, the gene product comprises at least two nucleotide-encoded peptide or protein tag sequences.

In alternative embodiments, the exogenous gene product or functional fragment thereof is an oligonucleotide sequence. In further embodiments, the exogenous gene product is an RNA molecule. In further embodiments, the exogenous gene product or functional fragment thereof is a ribozyme.

In certain embodiments wherein the genetically modified Mycoplasma bacterium comprises one or more exogenous gene products such as a protein, said protein is capable of interacting with a protein expressed by a distinct bacterium present in the host organism. In further embodiments, the distinct bacterium present in the host organism is a pathogenic bacterium, or considered as pathogenic in the art. In certain embodiments, the (pathogenic) distinct bacterium is present in the lung(s) of the host organism. In further embodiments, the protein capable of interacting with a distinct bacterium in the host organism is expressed on the surface of the Mycoplasma bacterium. In alternative embodiments, the protein capable of interacting with a distinct bacterium in the host organism is secreted by the Mycoplasma bacterium.

In certain embodiments, the genetically modified Mycoplasma bacterium is obtained by introducing said functional modification such as a deletion, insertion, and/or substitution in one or more genes of a Mycoplasma bacterium genome by recombinant DNA technology. In further embodiments, the recombinant DNA technology is a genome engineering method. In yet a further embodiment, the genome engineering method is a recombinase and/or nuclease-based genome engineering method. In certain embodiments, the genetically modified Mycoplasma bacterium is obtained by introducing said functional modification such as a deletion, insertion, and/or substitution in one or more genes of a Mycoplasma bacterium by template-mediated genome engineering. In further embodiments, the template-mediated genome engineering comprises a step of contacting the genome of a Mycoplasma bacterium with a nuclease and/or recombinase, and additionally providing an oligonucleotide sequence comprising homologous region flanking the desired genetic modification. In further embodiments, the oligonucleotide sequence is provided to the Mycoplasma bacteria simultaneously with a recombinase and/or nuclease. In certain embodiments, the genetically modified Mycoplasma bacterium is obtained by introducing said function modification such as a deletion, insertion, and/or substitution in one or more genes of a Mycoplasma bacterium genome by a designer nuclease. In further embodiments, the designer nuclease specifically targets the gene encoding, or the regulatory gene sequence regulating expression of a targeted gene product. Non-limiting examples of a suitable recombinase is the Bacillus subtilis gp35 recombinase as defined herein. Non-limiting examples of suitable designer nucleases are Zinc Finger Nucleases (ZFNs), Transcription Activator-Like Effector Nucleases (TALENs), mega nucleases, restriction enzymes, and Clustered Regularly Interspace Short Palindromic Repeats (CRISPR). Methods and suitable constructs using designer nucleases have been described in detail in the art (Gaj et al., ZFN, TALEN, CRISPR/Cas-based methods for genome engineering, Trends in Biotechnology, 2013). In yet further embodiments, the genetically modified Mycoplasma bacterium as described herein is obtained by a first step of integrating a desired genomic alteration by gp35-mediated recombination, followed by a counter selection step wherein a designer nuclease targeting the wild-type (unmodified) sequence of the targeted locus is expressed or introduced in the Mycoplasma bacterium. In such embodiments, any cleavage of unmodified yet targeted genomic regions in the Mycoplasma bacterium induces cell death of said Mycoplasma.

In certain embodiments, the genetically modified Mycoplasma bacterium described herein has a genomic sequence that is partially or completely obtained by chemical synthesis. Chemical synthesis of oligonucleotide sequences has been described in detail in the art on numerous occasions, for example by Laikhter and Linse, The chemical synthesis of oligonucleotides, Biosynthesis, 2014). A skilled person understands that a genome, a minimal genome, or a reorganized genome may be synthetically synthesized as has been described in the art (Gibson et al., Creation of a bacterial cell controlled by a chemically synthesized genome, Science, 2010). In certain embodiments, the genetically modified Mycoplasma bacterium as described herein is not able to propagate without a resource, such as a nutrient or anti-toxin that is not essential for a naturally occurring Mycoplasma bacterium. In certain embodiments, the genetically modified Mycoplasma bacterium depends on the provision of one or more non-naturally occurring amino acids and/or nucleotides in order to be able to propagate. In certain embodiments, genetically modified Mycoplasma bacterial strains have a rationally rearranged genome. Rearranged (bacterial) genomes have also been described in the art and the concept of rearranged genomes is therefore known to a skilled person (Bu et al., Rational construction of genome-reduced and high-efficient industrial Streptomyces chassis based on multiple comparative genomic approaches, Microbial cell factories, 2019).

In a further aspect, a kit of part comprising a live genetically modified Mycoplasma bacterium is envisaged. In further embodiments, said live genetically modified Mycoplasma bacterium comprises a genetic element facilitating genomic insertion of an exogenous nucleotide sequence. In further embodiments, the modified Mycoplasma bacterium comprises a genomic nucleotide arrangement encoding a recombinase and/or encoding a nuclease. In further embodiments, the modified Mycoplasma bacterium comprises a genomic nucleotide arrangement encoding a gp35 recombinase. In yet further embodiments the modified Mycoplasma bacterium comprises a genomic nucleotide arrangement encoding a protein with a sequence identity of at least 65%, at least 70%, at least 75%, at least 80%, preferably at least 85%, at least 90%, at least 95% to the amino acid sequence of the GP35 recombinase form Bacillus subtilis bacteriophage SPP1, annotated under NCBI reference sequence NP_690727.1. In alternative embodiments, the live genetically modified Mycoplasma bacterium comprises an extrachromosomal nucleotide arrangement encoding a recombinase, preferably a recombinase with a sequence identity of at least 65%, at least 70%, at least 75%, at least 80%, preferably at least 85%, at least 90%, at least 95% to the amino acid sequence of the GP35 recombinase from Bacillus subtilis bacteriophage SPP1, annotated under NCBI reference sequence NP_690727.1.

In a further aspect, a Mycoplasma bacterium as described herein for use as a medicament is intended. In certain embodiments, the Mycoplasma bacterium is a live Mycoplasma bacterium. In alternative embodiments, the Mycoplasma bacterium is not alive, or considered alive by a person skilled in the art, at the time when it is used as a medicament. In certain embodiments, the Mycoplasma bacterium is used as a medicament when lyophilized. In certain embodiments, two or more genetically modified Mycoplasma bacteria with a distinct genomic sequence are simultaneously used as a medicament. It is understood that a medicament as used in the context herein refers to a substance, or drug, that is used to diagnose, cure, treat, or prevent disease. In certain embodiments, a live genetically modified Mycoplasma bacterium that is attenuated by said modifications is used as a medicament, preferably wherein said Mycoplasma comprises a functional modification such as a deletion, insertion, and/or substitution in MPN133, and MPN372. In certain embodiments, a genetically modified Mycoplasma as described herein is used as an oral medicament. In further embodiments, a genetically modified Mycoplasma as described herein is used as a medicament configured for administration by inhalation.

“Lyophilized”, “freeze-dried”, or “cryodesiccated” can be used interchangeably herein and refer to a condition and/or state of a sample, formulation, or product obtained by means of lyophilisation. Lyophilisation is a dehydration process which involves freezing the product without destroying the physical structure of the matter. Lyophilisation comprises at least a freezing step and a sublimation step. The sublimation step may comprise two stages of drying: a primary drying step and a secondary drying step. Lyophilisation is commonly used in pharmaceutical manufacturing. In the freezing step, the material is cooled to a temperature wherein the solid, liquid, and gas phases of the material may exist. Pharmaceutical active ingredients or products may be lyophilized to achieve chemical stability enabling storage at room temperature. A method of lyophilisation differs from a conventional drying method that evaporates water using heat. Advantages of lyophilisation may be but are not limited to improved aseptic handling, enhanced stability of a dry powder, the removal of water without excessive heating of the product, and enhanced product stability in a dry state. In general, the quality of a rehydrated, lyophilized product is excellent and does not show inferior (therapeutic) characteristics to a non-lyophilized product.

In certain embodiments, the genetically modified Mycoplasma bacterium as described herein is used as a medicament to treat respiratory diseases. In further embodiments, the genetically modified Mycoplasma bacterium is used as a medicament to treat cystic fibrosis, chronic obstructive pulmonary disease, or ventilator-associated pneumoniae. In certain embodiments, a genetically modified Mycoplasma bacterium is used as a reservoir, container, or delivery vehicle of exogenous gene products, preferably therapeutic proteins. In certain embodiments where an alive genetically modified Mycoplasma bacterium is used, said bacterium is considered to be alive when it is able to at least locally produce or display a therapeutic or immunogenic protein, and preferably has the capacity to propagate in the host organism.

In certain aspects, a method of treatment of a disease using a genetically modified Mycoplasma bacterium as described herein is envisaged. In further aspects, a method of treating a subject diagnosed with a pulmonary disease or suspected to have a pulmonary disease is envisaged. In certain embodiments, the method comprises contacting the subject with a live genetically modified bacterium as described herein. In certain embodiments, the method comprises a step of orally or nasally administering a live or lyophilized genetically modified Mycoplasma bacterium to a subject. In further embodiments, the method comprises administration of a live or lyophilized genetically modified Mycoplasma bacterium to a subject by means of inhalation, for example by use of an inhalator. In certain embodiments, the method of treatment comprises a single administration of a genetically modified Mycoplasma bacterium as described herein to a subject. In alternative embodiments, the method of treatment comprises a periodical administration of a genetically modified Mycoplasma bacterium as described herein to a subject. In further embodiments, distinct genetically modified Mycoplasma are administered to a subject at different points in time. In certain embodiments, the live genetically modified Mycoplasma bacteria are administered to a subject diagnosed to have a respiratory infection. In further embodiments, a modified Mycoplasma bacterium as described herein is used as part of combination therapy in a subject diagnosed with a respiratory infection.

In yet a further aspect, a Mycoplasma bacterium as described herein for use as a vaccine is intended. In certain embodiments, a live genetically modified Mycoplasma bacterium as described herein is used as a vaccine. In certain embodiments, said modified Mycoplasma bacterium comprises an exogenous gene product on its surface. In alternative embodiments, said Mycoplasma bacterium secretes an exogenous gene product. In certain embodiments, a genetically modified Mycoplasma bacterium as described in any embodiment herein is used as a vaccine for respiratory infections. In certain embodiments, a genetically modified Mycoplasma bacterium as described herein is used as a vaccine, wherein said vaccine additionally comprises substances, live or inactivated pathogen, or immunogenic proteins suitable for eliciting an immune response in the subject undergoing vaccination for a distinct disease. In certain embodiments, said vaccine further comprises an immunologic adjuvant, such as but by no means limited to alum. Incorporation of adjuvants in vaccines is commonly used in the art and is therefore known to a person skilled in the art (e.g. Petrovsky, Comparative safety of vaccine adjuvants: a summary of current evidence and future needs, Drug Safety, 2015, and Del Giudice et al., Correlates of adjuvanticity: A review on adjuvants in licensed vaccines, Seminar in Immunology, 2018). In a further aspect, use of a genetically modified bacterium as described herein, for the manufacture of a medicament is envisaged. In certain embodiments, use of a genetically modified bacterium as described herein for the manufacture of a medicament for the prevention or treatment of respiratory infection is envisaged.

In certain embodiments wherein the genetically modified Mycoplasma bacterium is used as a vaccine, said Mycoplasma bacterium displays at least one exogenous proteogenic sequence on its surface. The length, sequence composition, or origin of the exogenous proteogenic sequence are not particularly restricted in the context of the invention. Preferably, said proteogenic sequence is capable of eliciting an immunogenic response in the subject acting as recipient of the genetically modified Mycoplasma bacterium. Such sequences are commonly indicated in the art as “antigenic sequences” or “antigens”. The term “exogenous” used herein may therefore be a peptide fragment of any protein or a further mutagenized peptide fragment thereof. The protein may be expressed by one or more Mycoplasma species, or any other organism. Hence, it is envisaged that the genetically modified Mycoplasma bacterium described herein may act as a means to present any antigenic peptides to a recipient. The recipient preferably is a mammal, such as but not limited to humans, cattle, and domestic animals. In certain embodiments, the genetically modified Mycoplasma described herein may display more than one, preferably at least 2, preferably at least 3, preferably at least 4, preferably at least 5, preferably more than 5 exogenous proteogenic sequences. In further embodiments, the more than one exogenous proteogenic sequence are displayed as a fusion peptide on the surface of the genetically modified Mycoplasma bacterium. Evidently, the number of exogenous proteogenic sequences is not particularly limited, and even do not need to stem from the same organism.

In a further aspect, a genetically modified Mycoplasma bacterium as described herein for use to modulate the composition of a lung microbiome in a subject is intended. In certain embodiments, the composition of the lung microbiome in a subject after use of said Mycoplasma is characterized by an increase in relative Mycoplasma concentration of at least 10%, preferably at least 20%, at least 30%, at least 40%, at least 50%, at least 60%, at least 70%, compared to the relative amount of Mycoplasma bacteria in the lung microbiome of said subject prior to treatment. A skilled person is aware that a change in lung microbiome composition may drastically affect the health condition of a subject, as has been described on several occasions in the art (inter alia in O’Dwyer et al., The Lung Microbiome, Immunity and the Pathogenesis of Chronic Lung Disease, Journal of immunology, 2016). In certain embodiments, a genetically modified Mycoplasma bacterium as described herein is envisaged to modulate the composition of a lung microbiome, wherein the change in composition reduces the relative amount of one or more pathogenic bacteria below a threshold needed to establish symptoms of pathology.

In another aspect, envisaged herein is a method of producing an attenuated Mycoplasma bacterium, wherein the method comprises introducing an inactivating modification in at least two genes or operons encoding a gene product independently selected from the group consisting of: cytoadherence proteins, lipid synthesis enzymes producing immunogenic products, oxidoreductases, nucleases, toxins, lipoproteins, inflammatory regulating proteins, immunogenic proteins, or cancer inducing proteins. A skilled person is aware that certain gene products described in the art may be classified under more than one category cited above. In further embodiments, the inactivating modifications described herein are introduced in a live Mycoplasma bacterium with a site-directed recombinase, via random transposon insertion, and/or a site-directed nuclease. In certain embodiments, the inactivating modification is introduced by a nuclease. In alternative embodiments, the inactivating modification is introduced by a recombinase. In yet alternative embodiments, the inactivating modification is introduced by a catalytically inactive nuclease fused to a catalytic protein or fragment of a protein comprising a nuclease function. In certain embodiments, the inactivating modification is introduced by contacting a Mycoplasma bacterium with a recombinase or nuclease, and in addition providing said Mycoplasma bacterium with a nucleotide sequence comprising a desired modification flanked by two nucleotide regions homologous to the regions flanking the targeted nucleotide sequence in the genomic sequence of the Mycoplasma bacterium. In certain embodiments, the method further comprises a step of selecting genetically modified Mycoplasma bacteria. In further embodiments, the selection step is based on a phenotypic trait displayed by Mycoplasma bacteria comprising the desired alteration. In certain further embodiments, the phenotypic trait confers (is) (increased) resistance to one or more antibiotics when compared to a Mycoplasma bacterium not comprising said alteration. In alternative further embodiments, the phenotypic trait is characterized by the expression of one or more fluorescent proteins. In yet alternative further embodiments, the phenotypic trait is a change in growth morphology.

In further embodiments, the method further comprises a step of providing a synthetic genome, or a portion thereof, and transferring said (portion of) the synthetic genome to a naturally occurring Mycoplasma bacterium. In certain embodiments, the complete synthetic genome is synthesized in vitro prior to transfer to the Mycoplasma bacterium. In alternative embodiments, the synthetic genome is synthesized in different segments or portions and are ligated after transfer to the Mycoplasma bacterium. In certain embodiments, the synthetic genome exists in parallel with the original genome of the Mycoplasma bacteria. In further embodiments, the method further comprises a step of inactivating and/or removing the original genome of said live Mycoplasma bacterium. In preferred embodiments, the inactivation of the original genome of said live Mycoplasma bacterium comprises degradation of said original genome. Exemplary methods for replacing complete genomes have been described in the art (Lartigue et al., Genome transplantation in bacteria: changing on species to another, Science, 2007).

In a further aspect, the current disclosure envisages the use of an attenuated Mycoplasma bacterium as described herein for the production of at least one exogenous gene product or fragment thereof. In certain embodiments, the exogenous gene product is produced by said Mycoplasma bacterium in a host organism. In alternative embodiments the Mycoplasma bacterium is used for the production of at least one exogenous gene product in a bio production vessel, including but not limited to a bioreactor or a fermenter. In certain embodiments, the exogenous gene product is a biological. As used herein, the meaning of the term “biological” is used according to the commonly accepted definition found in the art, i.e. a substance of biological origin used as a drug, vaccine, or pesticide. Emphasis is added that the genetically modified Mycoplasma bacteria may be used for use as a biological as such, or that the modified Mycoplasma bacteria may solely be used for the production of a biological, or as a combination thereof.

In a different aspect, a pharmaceutical composition comprising the genetically modified Mycoplasma bacterium as described herein is envisaged. In certain embodiments, the pharmaceutical composition comprises water for injection or a physiological saline solution. A person skilled in the art appreciates that the terms “pharmaceutical composition”, “pharmaceutical formulation”, and “pharmaceutical preparation” can be used interchangeably herein and are meant to describe compositions containing a genetically modified Mycoplasma bacterium as active pharmaceutical ingredient, formulated with a pharmaceutically acceptable excipient, and manufactured or sold with the approval of a governmental regulatory agency as part of a therapeutic regimen for the treatment of disease in a mammal. It is evident that pharmaceutical compositions are indicative for those compositions that comprise a therapeutically effective amount of genetically modified Mycoplasma bacteria, or at least an amount of genetically modified Mycoplasma bacteria that, when introduced into a host organism as live bacteria, can propagate to express or deliver a therapeutically effective amount of a desired gene product and/or bacterial cargo.

The term “therapeutically effective amount” as used herein, refers to an amount of active compound or pharmaceutical agent that elicits the biological or medicinal response in a subject that is being sought by a researcher, veterinarian, medical doctor or other clinician, which may include a reduction or complete removal of the symptoms associated with the disease or condition being treated. Methods to determine pharmaceutically effective amounts are known in the art and are therefore known to a skilled person. It is further evident that therapeutic effective amounts are determined in function of the specific subject in need of treatment. Further, a wording such as “a subject in need of treatment” includes any subject or group of subjects that would benefit from treatment of a given condition. Such subjects may include, without limitation, those that have been diagnosed with a condition susceptible to treatment with the genetically modified Mycoplasma bacterium, those prone to develop said condition and/or those in who said condition is to be prevented.

The terms “treat” or “treatment” encompass both the therapeutic treatment of an already developed disease or condition, such as the therapy of an already developed pulmonary disease, as well as prophylactic or preventive measures, wherein the aim is to prevent or lessen the chances of incidence of an undesired affliction, such as to prevent occurrence, development and progression of a pulmonary infection. Beneficial or desired clinical results may include, without limitation, alleviation of one or more symptoms or one or more biological markers, diminishment of extent of disease, stabilized (i.e., not worsening) state of disease, delay or slowing of disease progression, amelioration or palliation of the disease state, and the like. “Treatment” can also mean prolonging survival as compared to expected survival if not receiving treatment.

As used herein, the terms “therapeutic treatment” or “therapy” and the like, refer to treatments wherein the object is to bring a subjects body or an element thereof from an undesired physiological change or disorder, including but not limited to pulmonary infections, to a desired state, such as a less severe or unpleasant state (e.g., amelioration or palliation), or back to its normal, healthy state (e.g., restoring the health, the physical integrity and the physical well-being of a subject), to keep it (i.e., not worsening) at said undesired physiological change or disorder (e.g., stabilization), or to prevent or slow down progression to a more severe or worse state compared to said undesired physiological change or disorder.

In certain embodiments, the pharmaceutical formulation further comprises one or more further pharmaceutical active ingredients. In certain embodiments, the pharmaceutical formulation further comprises one or more non-active pharmaceutical ingredients or inactive ingredients, commonly referred to in the art as excipients. In further embodiments, the pharmaceutical composition may be a lyophilized pharmaceutical composition.

The term “excipient”, commonly termed “carrier” in the art may be indicative for all solvents, including but by no means limited to: diluents, buffers (e.g., neutral buffered saline, phosphate buffered saline, or optionally Tris-HCl, acetate or phosphate buffers), solubilisers (e.g., Tween 80, Polysorbate 80), colloids, dispersion media, vehicles, fillers, chelating agents (e.g., EDTA or glutathione), amino acids (e.g., glycine), proteins, disintegrants, binders, lubricants, wetting agents, stabilizers, emulsifiers, sweeteners, colorants, flavorings, aromatisers, thickeners, agents for achieving a depot effect, coatings, antifungal agents, preservatives (e.g., benzyl alcohol), antioxidants (such as, e.g., ascorbic acid, sodium metabisulfite), tonicity controlling agents, absorption delaying agents, adjuvants, bulking agents (e.g., lactose, mannitol) and the like. The use of such media and agents for formulating pharmaceutical compositions is well known in the art.

The term “subject”, “patient”, and “subject in need” may be used interchangeably herein and refer to animals, preferably warm-blooded animals, more preferably vertebrates, and even more preferably mammals specifically including humans and non-human mammals. The term “mammals”, or “mammalian subjects” refers to any animal classified as such and hence include, but are not limited to humans, domestic animals, commercial animals, farm animals, zoo animals, sport animals, pet and experimental animals such as dogs, cats, guinea pigs, rabbits, rats, mice, horses, cattle, cows; primates such as apes, monkeys, orangutans, and chimpanzees; canids such as dogs and wolves; felids such as cats, lions, and tigers; equids such as horses, donkeys, and zebras; food animals such as cows, pigs, and sheep; ungulates such as deer and giraffes; rodents such as mice, rats, hamsters and guinea pigs; and so on. Preferred patients are human subjects. Particularly preferred are human subjects, including both genders and all age categories thereof.

In certain embodiments, the pharmaceutical composition is a lyophilized composition that may need to be reconstituted prior to administration. In further embodiments, the pharmaceutical composition can be formulated into a unit dosage form, including but not limited to hard capsules, soft capsules, tablets, coated tablets such as lacquered tablets or sugar-coated tablets, granules, aqueous or oily solutions, syrups, emulsions, suspensions, ointments, pastes, lotions, gels, inhalants or suppositories, which may be provided in any suitable packaging means known in the art, non-limiting examples being troches, sachets, pouches, bottles, films, sprays, microcapsules, implants, rods or blister packs.

Additionally, the disclosure provides the following statements:

Statement 1. A genetically modified Mycoplasma bacterium, wherein said Mycoplasma bacterium comprises in its genome a functional modification in Ca2+ dependent cytotoxic nuclease gene (MPN133) and ADP-ribosyltransferase CARDS gene (MPN372), and wherein said functional modifications attenuate said Mycoplasma bacterium.

Statement 2. The genetically modified Mycoplasma bacterium according to statement 1, further comprising a functional modification in at least one gene, preferably at least two genes selected from the group consisting of Table 1.

Statement 3. The genetically modified Mycoplasma bacterium according to any one of the preceding statements, wherein said bacterium further comprises a functional modification in a gene encoding a peroxide producing protein, preferably in a gene encoding glycerol 3-phosphate oxidase, more preferably encoding glycerol-3-phospate dehydrogenase (MPN051).

Statement 4. The genetically modified Mycoplasma bacterium according to any one of the preceding statements, wherein said bacterium further comprises a functional modification in a gene encoding a second (surface) nuclease, preferably encoding membrane nuclease A (MPN491).

Statement 5. The genetically modified Mycoplasma bacterium according to any one of the preceding statements, wherein said bacterium comprises a functional modification in one or more genes encoding for a cytoadherence protein, preferably selected from the group consisting of: MPN 141, MPN142, MPN453, MPN447, MPN310, MPN452, MPN309.

Statement 6. The genetically modified Mycoplasma bacterium according any one of the preceding statements, wherein said bacterium further comprises a functional modification in a gene encoding an immunogenic protein that is capable of eliciting an immune response in a host organism, preferably in a gene encoding conserved hypothetical protein MPN_400 (MPN400).

Statement 7. The genetically modified Mycoplasma bacterium according to any one of the preceding statements, wherein said bacterium further comprises a functional modification in one or more genes encoding a protein capable of eliciting Guillain-Barre in a host organism, preferably encoding UDP-glucose 4-epimerase (MPN257) and/or glycosyltransferase (MPN483).

Statement 8. The genetically modified Mycoplasma bacterium according to any one of the preceding statements, wherein said bacterium further comprises a functional modification in a gene encoding a protein that inhibits growth of said bacterium in a bioreactor, preferably encoding a protein similar to intracellular protease ThiJ/PfpI (MPN294).

Statement 9. The genetically modified Mycoplasma bacterium according to any one of the preceding statements, wherein said bacterium further comprises a functional modification in one or more genes encoding a lipoprotein, preferably selected from the group consisting of: MPN141, MPN142, MPN152, MPN162, MPN199, MPN200, MPN224, MPN233, MPN271, MPN284, MPN288, MPN293, MPN333, MPN372, MPN415, MPN447, MPN592, MPN597, MPN602, MPN611, MPN011, MPN052, MPN054, MPN058, MPN083, MPN084, MPN097, MPN098, MPN363, MPN369, MPN408, MPN411, MPN436, MPN439, MPN442, MPN444, MPN456, MPN467, MPN489, MPN506, MPN523, MPN582, MPN585, MPN586, MPN587, MPN588, MPN590, MPN591, MPN592, MPN639, MPN640, MPN641, MPN642, MPN643, MPN644, MPN645, MPN646, MPN647, MPN648, MPN649, MPN650, MPN654.

Statement 10. The genetically modified Mycoplasma bacterium according to any one of the preceding statements, wherein said bacterium comprises a functional modification in prolipoprotein diacylglyceryl transferase gene MPN224 and lipoprotein signal peptidase gene MPN293.

Statement 11. The genetically modified Mycoplasma bacterium according to any one of the preceding statements, wherein said bacterium further comprises a functional modification in a gene encoding an oncogenic protein, preferably encoding high affinity transport system protein p37 (MPN415).

Statement 12. The genetically modified Mycoplasma bacterium according to any one of the preceding statements, wherein said bacterium further comprises a functional modification in a gene encoding an RNA polymerase factor, preferably encoding probable RNA polymerase sigma-D factor (MPN626).

Statement 13. The genetically modified Mycoplasma bacterium according to any one of the preceding statements, wherein said bacterium further comprises a functional modification in one or more genes encoding a secreted Mycoplasma gene product, preferably selected from the group consisting of: MPN400, MPN036, MPN592, MPN509, MPN647, MPN084, MPN625, MPN213, MPN489, MPN142, MPN444, MPN642, MPN398, MPN491, MPN083, MPN141.

Statement 14. The genetically modified Mycoplasma bacteria according to any one of the preceding statements, wherein said functional modification refers to insertion, deletion, substitution, or any combination thereof of one or more nucleotides in said genes.

Statement 15. The genetically modified Mycoplasma bacterium according to any one of the preceding statements, wherein attenuation indicates a reduction of toxicity by at least 30%, preferably at least 50%, more preferably at least 75%, most preferably at least 90%, when said bacterium is introduced to a host organism, preferably introduced in the respiratory system of said host organism, when compared to a reference Mycoplasma bacterium, wherein said reference Mycoplasma bacterium is a naturally occurring (wild type) Mycoplasma bacterium not comprising a genetic functional modification.

Statement 16. The genetically modified Mycoplasma bacterium according to any one of the preceding statements, wherein said Mycoplasma bacterium is a Mycoplasma pneumoniae bacterium.

Statement 17. The genetically modified Mycoplasma bacterium according to any one of the preceding statements, wherein said Mycoplasma bacterium comprises a nucleotide sequence encoding an exogenous gene product or a functional fragment thereof, preferably wherein said nucleotide sequence is comprised in the genomic sequence of said Mycoplasma bacterium.

Statement 18. The genetically modified Mycoplasma bacterium according to the previous statement, wherein said exogenous gene product or functional fragment thereof is a protein, preferably a therapeutic protein, a protein involved in specific attachment to a host protein, an enzyme, immunogenic protein, or DNA-binding protein, more preferably wherein said therapeutic or immunogenic protein is expressed on the surface of said Mycoplasma bacterium and/or is secreted by said Mycoplasma bacterium.

Statement 19. The genetically modified Mycoplasma bacterium according to any one of the preceding statements, wherein said bacterium is obtained by introducing said functional modification in one or more genes of a Mycoplasma bacterium genome by recombinant DNA technology, preferably by a genome engineering method, more preferably by a recombinase and/or nuclease-based genome engineering method.

Statement 20. The genetically modified Mycoplasma bacterium according to any one of the preceding statements, wherein said genome comprising one or more functional modifications is partially, preferably completely obtained by chemical synthesis.

Statement 21. A Mycoplasma bacterium according to any one of statements 1 to 20, for use as a medicament.

Statement 22. A Mycoplasma bacterium according to any one of statements 1 to 20, for use as a vaccine.

Statement 23. A Mycoplasma bacterium according to any one of statements 1 to 20, for use to modulate the composition of a lung microbiome in a subject.

Statement 24. A method of producing an attenuated Mycoplasma bacterium, wherein the method comprises introducing a functional modification in at least two genes encoding a gene product independently selected from the group consisting of: cytoadherence proteins, oxidoreductases, nucleases, toxins, lipoproteins, inflammatory regulating proteins, immunogenic proteins, or cancer inducing proteins.

Statement 25. The method according to statement 24, wherein said functional modifications are introduced in a live Mycoplasma bacterium with a site-directed recombinase and/or a site-directed nuclease.

Statement 26. The method according to statement 24 or 25, wherein the method comprises a step of providing a synthetic genome, or a portion thereof, and transferring said (portion of) the synthetic genome to a naturally occurring Mycoplasma bacterium.

Statement 27. The method according to statement 26, wherein the method further comprises a step of inactivating, preferably degrading, and/or removing the original genome of said live Mycoplasma bacterium.

Statement 28. Use of an attenuated Mycoplasma bacterium of any of statements 1 to 20 for the production of at least one exogenous gene product or fragment thereof.

Statement 29. A pharmaceutical composition comprising the genetically modified Mycoplasma bacterium according to any one of statements 1 to 20.

While the invention has been described in conjunction with specific embodiments thereof, it is evident that many alternatives, modifications, and variations will be apparent to those skilled in the art in light of the foregoing description. Accordingly, it is intended to embrace all such alternatives, modifications, and variations as follows in the spirit and broad scope of the appended claims. The herein disclosed aspects, embodiments, and statements of the invention are further supported by the following non-limiting examples.

### EXAMPLES

### 1. Method for Producing Modified Mycoplasma Strains

0.5 nmol of different editing oligo’s as described herein were co-transformed with pUC57PuroSelector plasmid into a M. pneumoniae strain expressing GP35 from a constitutive promoter and Cre recombinase from the inducible Ptet promoter. A mock transformation without oligo served as control condition to monitor non-specific plasmid integration. After transformation, cells were allowed to recover for at least 3 hours in Hayflick medium at 37° C.

Cre recombinase was transiently expressed to mediate the integration of pUC57PuroSelector plasmid which allows for selection of edited clones. Therefore, the complete amount of the oligo+ plasmid co-transformations was inoculated into T75 flasks containing 25 ml of Hayflick medium supplemented with 5 ng/ml of anhydrotetracycline and 3 µg/ ml puromycin. Cultures were allowed to grow in the presence of inducer for a period of time of minimum 12 hours and maximum 72 hours. Afterwards, cells were removed from the flasks in 500 µl of Hayflick medium, and half of this volume was spread onto Hayflick 0.8% bacto agar plates containing 3 µg/ml puromycin. Plates were incubated at 37° C. and 5% CO2 for a minimum period of 10 days before screening of the resulting colonies.

In an alternative approach, Cre expression was induced directly on the bacto agar plates. In this methodology, after a recovery phase following the co-transformations, half of the volume is spread onto Hayflick 0.8% bacto agar plates supplemented with 3 µg/ml puromycin and 1 ng/ml anhydrotetracycline. Plates were incubated at 37° C. and 5% CO2 for a minimum period of 10 days before screening of the resulting colonies.

Alternatively, this method allows carrying out gene complementation. For instance, MPN483 coding for the main glycosyltransferase found in M. pneumoniae genome was deleted with an editing oligo. Subsequently, the deletion was complemented with tailored versions of pUC57PuroSelector plasmid, in which genes coding for different glycosyltransferases activities were cloned.

Similarly, MPN489 coding for an essential lipoprotein can be deleted with an editing oligo and viability of the cell may be rescued by using tailored versions of pUC57PuroSelector plasmid carrying modified versions of MPN489 that would preclude the acylation of the protein. Specific genes are discussed in section 2.

### 2. Rational Design of Modified Mycoplasma Strains

Through extensive experimentation, the inventors have identified several genetically modified Mycoplasma strains that have improved characteristics. In principle, an optimal genetically modified Mycoplasma strain should address multiple issues that can be envisaged when using Mycoplasma as a delivery vehicle or chassis for locally producing exogenous gene products such as therapeutic proteins. These hurdles and solutions are described in detail in the following sections. Different genetically modified Mycoplasma strains are indicated herein with the annotation “Chassis Version”, abbreviated as “CV”, followed by an integer. Each CV is summarized in the overview table in section 2.9 (Table 7), which also summarizes the genetic background of each strain. Several chassis described herein express the heterologous gp35 recombinase, which is used to facilitate genetic modification as shown and discussed elaborately in a co-pending application. A skilled person understands that the gp35 recombinase or expression thereof is not a prerequisite for obtaining or using the modified Mycoplasma strains, and both chassis versions with or without gp35 are envisaged genetically modified Mycoplasma strains suitable for delivery or producing locally desired products.

### 2.1. Cytoadherence

The adhesion of Mycoplasma (pneumoniae) onto the respiratory epithelia involves cytadherence of the organism to sialoglycoproteins and sulphated glycolipids of the epithelial cells by a specialized organelle composed of adhesins and accessory proteins (Waites et al., New insights into the pathogenesis and detection of Mycoplasma pneumoniae infections, Future microbiology, 2008). The major proteins, that have been proven to be directly involved in receptor binding in the art, are the P1 (MPN142) and the P30 (MPN453) adhesins (Chourasia et al., Delineation of immunodominant and cytadherence segment(s) of Mycoplasma pneumoniae P1 gene, BMC microbiology, 2014). The P1-adhesin is a transmembrane protein, concentrated primarily at the tip of the attachment organelle of Mycoplasma (pneumoniae). Mutant strains lacking P1 fail to adhere to animal cells and are avirulent. The P1 adhesin needs of other auxiliary proteins: P30 (MPN453) adhesion factor-related protein A (72 kDa), B (85 kDa) and C (37 kDa), HMW 1-3 polypeptides (MPN447, MPN310, MPN452), P40 and P90 (MPN142) and P65 (MPN309). All these proteins are non-essential genes or genes contributing to bacterial fitness.

The inventors have generated several Mycoplasma strains comprising modified or inactivated MPN142 and/or MPN453 genes with the objective of improving three aspects in the chassis design:

1) Avoid attachment in order to improve chassis growth in suspension for production in fermenter. Also, removal of these accessory proteins decreases the overall metabolic load and the obtained chassis may grow faster than compared to Mycoplasma strains wherein the above genes are still expressed.

2) Reduction of pathogenicity by decreasing the attachment of the chassis to lung epithelial cell lines of the subject that is to be treated with a modified Mycoplasma bacterium subject of the present invention. However, a decrease in attachment potency could also imply that the chassis cells are cleared faster from the infected subject. Consequently, a reduction in the efficacy of the Mycoplasma product and/or a higher required minimum dose are hypothetical drawbacks hereof. Furthermore, an increase of the required dose would additionally imply an increased production cost. Thus, this aspect needs to be evaluated on a case-by-case basis and characterized for each envisaged application to design and arrive at an optimal chassis. For example, in a context of vaccination, using Mycoplasma strains unable to attach to lung epithelial cells of the subject could promote a better recognition by the immune system and enhance the response and the protection. On the other hand, for example in a context of treating other diseases like cancer where continuous and local delivery of therapeutic products is important, adherence to the tumour cells could be required. Hence, the potential merits of imparting changes to the adherence properties of the Mycoplasma chassis described herein needs to be assessed on a case-by-case basis.

3) Directing (i.e. targeting, homing) the chassis to specific cell types (e.g. to pathogenic bacteria or tumour cells residing in the host organism).

Three combinations of cytoadherence gene modifications are of particular interest, one that keeps the adhesion machinery intact which will ensure retention in the human lungs in order to secrete any active therapeutic products in the lung tissue (e.g. CV16). In a second strain, MPN142 or MPN453 genes have been inactivated or depleted (e.g. CV19). This strain could be used to generate further strains that expose antigens on the surface (for vaccination) or alternatively expose heterologous proteins that would bind to the pathogenic bacteria causing a lung infection. In this last scenario, the chassis could then be homed specifically to the focus of the infection where it causes local delivery in the site of action of therapeutic components (e.g. CV3). In addition, once the infection is resolved, the chassis will be washed away. Third, strains expressing a heterologous protein that will bind to a specific protein of a target human or animal cell in the lungs can be generated. This allows development of Mycoplasma infection models in animals normally not infected by this bacterium and could be used to study pathogenesis, drug delivery, etc. In accordance with the above, the merits of selecting an appropriate heterologous protein that allows an expansion or change in the host organisms that can be infected need to be carefully assessed on a case-by-case basis.

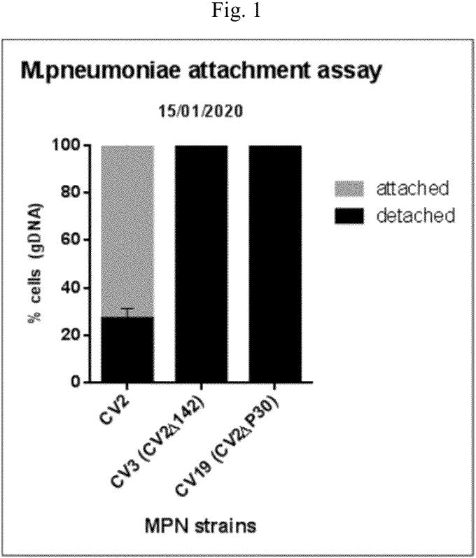

The inventors have developed and characterized the exemplary strains CV16 (CV0 strain (wild type with gp35 gene) with depletion of mpn453) CV19 (CV2 strain (attenuated strain, see below) with depletion of mpn453) and CV3 (CV2 strain with the depletion of mpn142). Results of a qPCR-based attachment assay for these strains are shown in FIG. 1.

Study of the proteome of the CV16 mutant (ΔP30 mpn453) vs the WT strains showed a significant decrease of the attachment p65 protein (MPN309), plus many other changes affecting ribosomal proteins and lipoproteins. Thus, depletion of MPN453 can have effects in the structure of the terminal organelle and affect other key proteins related with attachment.

Growth rates of different strains were measured by determining the number of bacteria at different time points by qPCR. Remarkably, CV19 and CV16 strains showed higher total number of bacteria and biomass than the respective reference strains. Thus, depletion of MPN453 also promotes growth and improves the efficiency of large-scale production in a bioreactor such as a fermenter. Optionally, expression of MPN453 can be repressed when growing in the fermenter and subsequently induced prior to inoculation in the lungs of a host organism such as a patient.

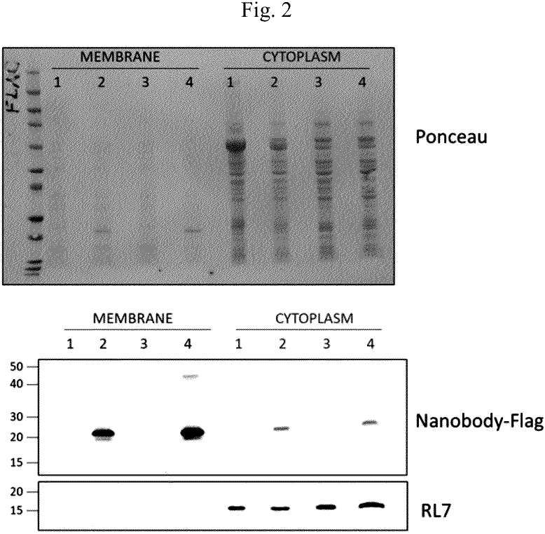

Thus, removal of endogenous membrane proteins allows for an increase in the production of heterologous secreted proteins. Western Blot experiments suggest that removal of P30 helps in having more heterologous protein on the membrane of the modified Mycoplasma bacterium (FIG. 2). These results are highly relevant for further improvement of modified Mycoplasma strains to deliver therapeutic proteins in the lungs or use as vaccine since this indicates a higher efficiency, which translates in practice to a lower amount of bacteria that are required to achieve a certain (clinical) effect, and consequently indicates that lower production volumes will be needed, ultimately leading to a reduction in manufacturing costs.

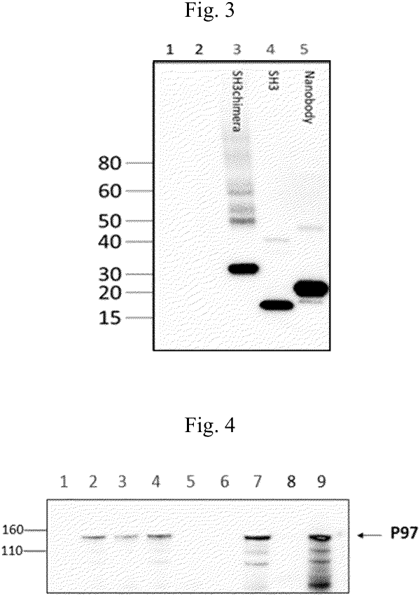

In addition, experimental data proves that heterologous proteins expressed at the surface of the modified Mycoplasma bacteria are able to recognize proteins in other bacteria. The first example is the SH3 domain of a phage bacteriolysin that specifically recognizes the surface of Staphylococcus aureus. The SH3 domain can be expressed as such, and as a chimeric protein that comprises two SH3 domains. In addition, to direct the chassis against Pseudomonas aeruginosa a nanobody can be expressed on the Mycoplasma surface which is directed against the flagellum of this bacterium (FIGS. 2 and 3).

Additionally, other adhesins from other Mycoplasma species have been expressed in the chassis to promote binding into the lungs of animal models for doing in vivo assays. For example, the Vsa protein from Mycoplasma pulmonis (which infects mice) and the P97 protein from Mycoplasma hyopneumoniae (that infects pigs, FIG. 4) can be expressed on the surface of the Mycoplasma strains to promote the binding of said strains to epithelial cell lines of mice and pigs, respectively.

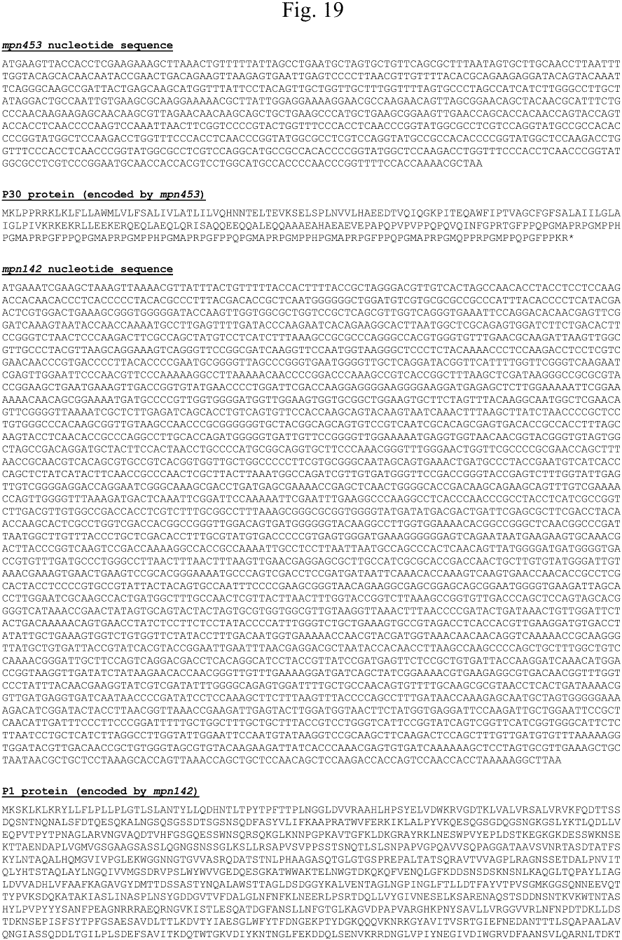

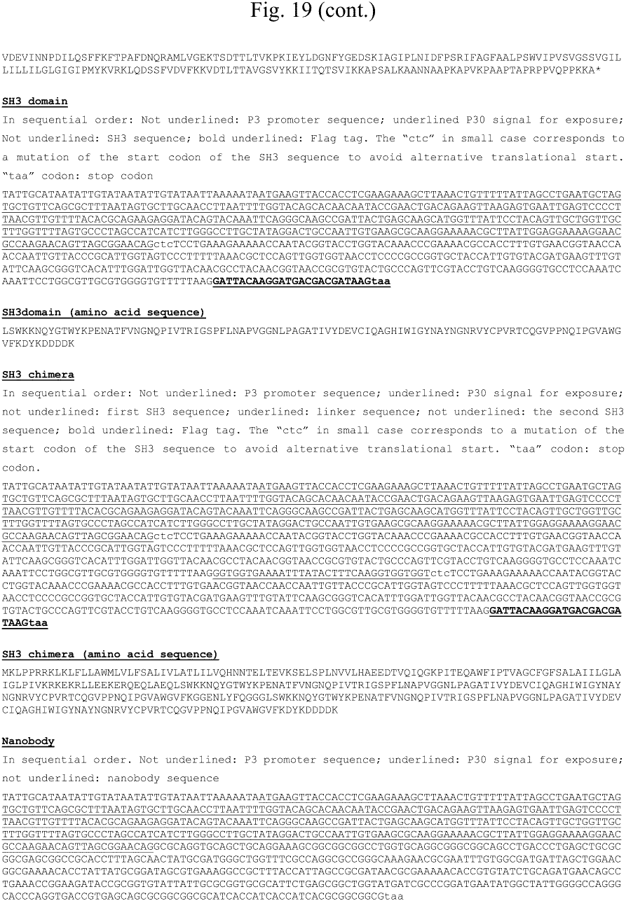

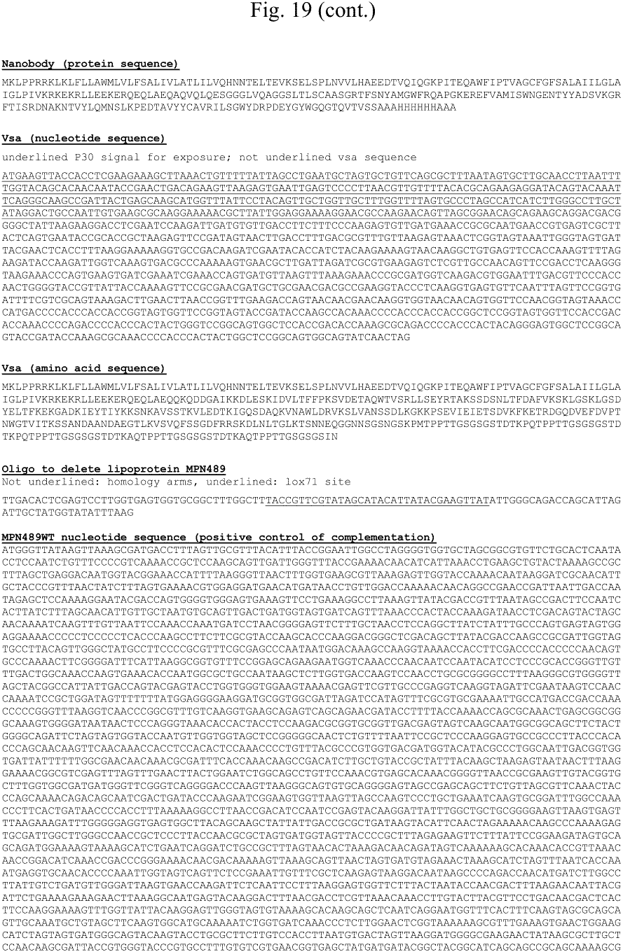

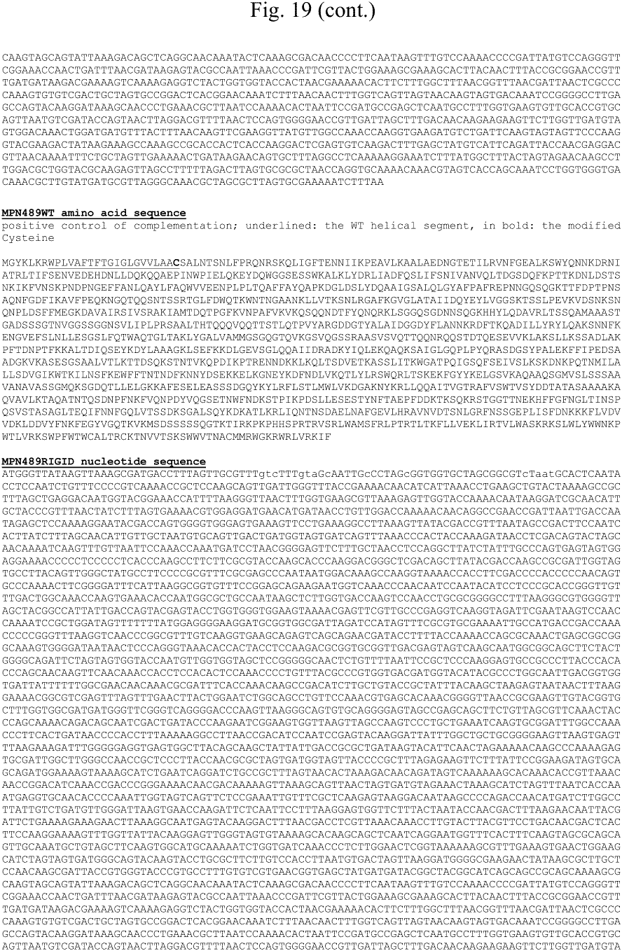

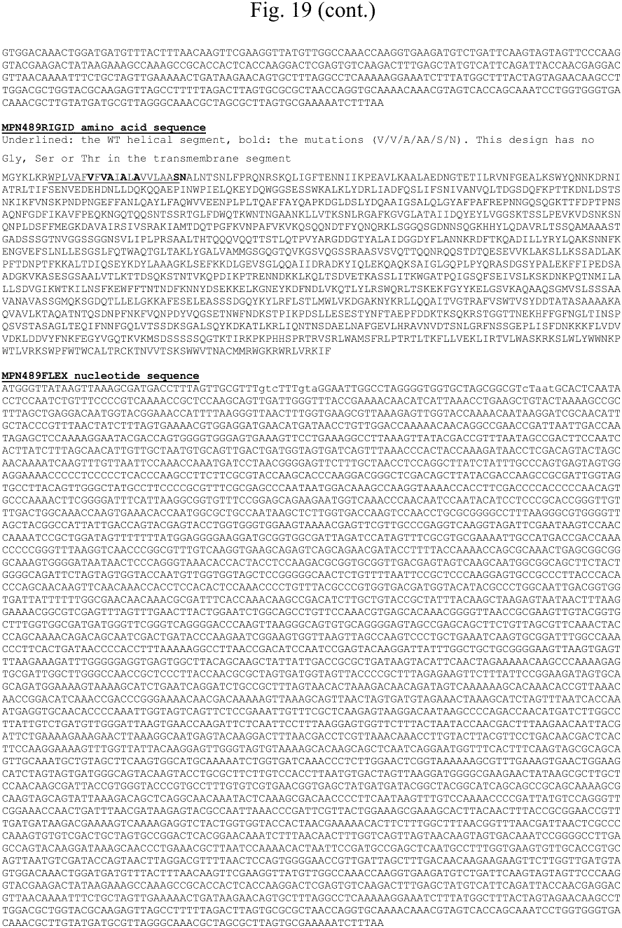

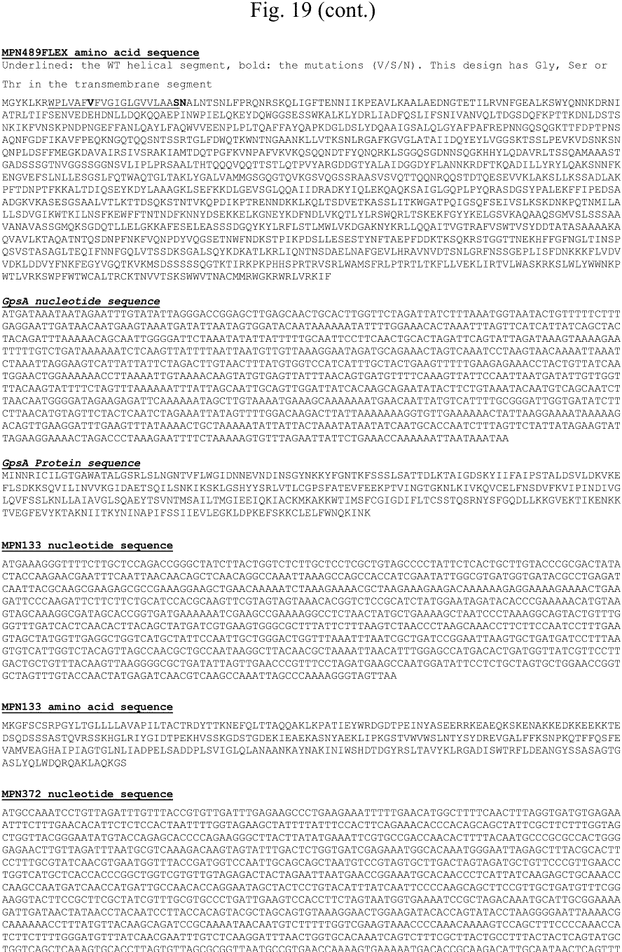

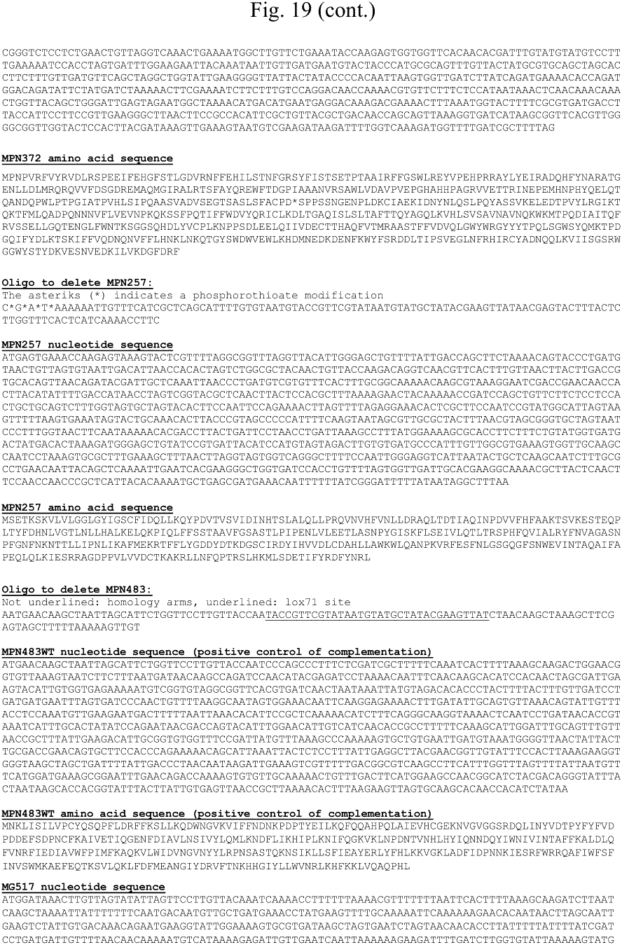

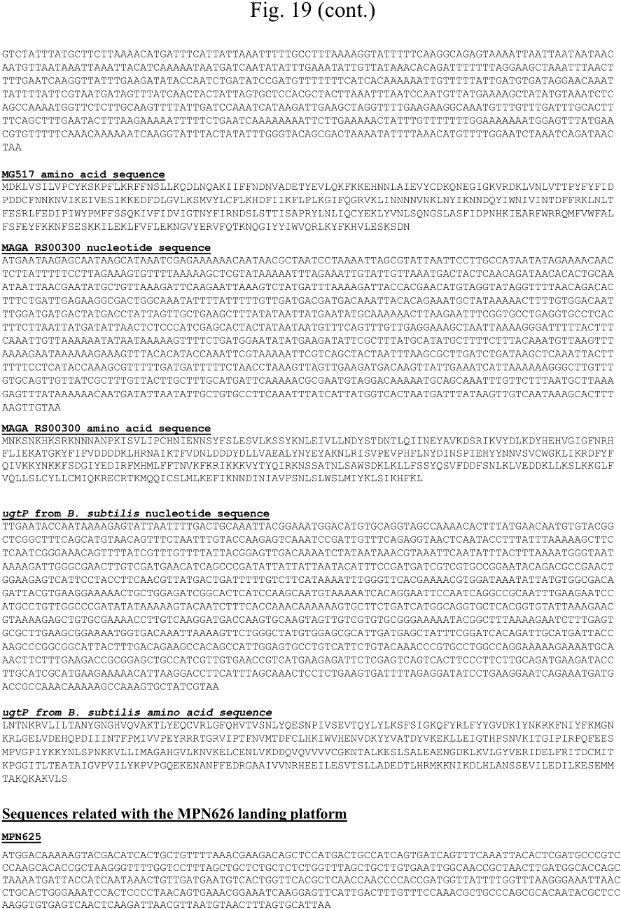

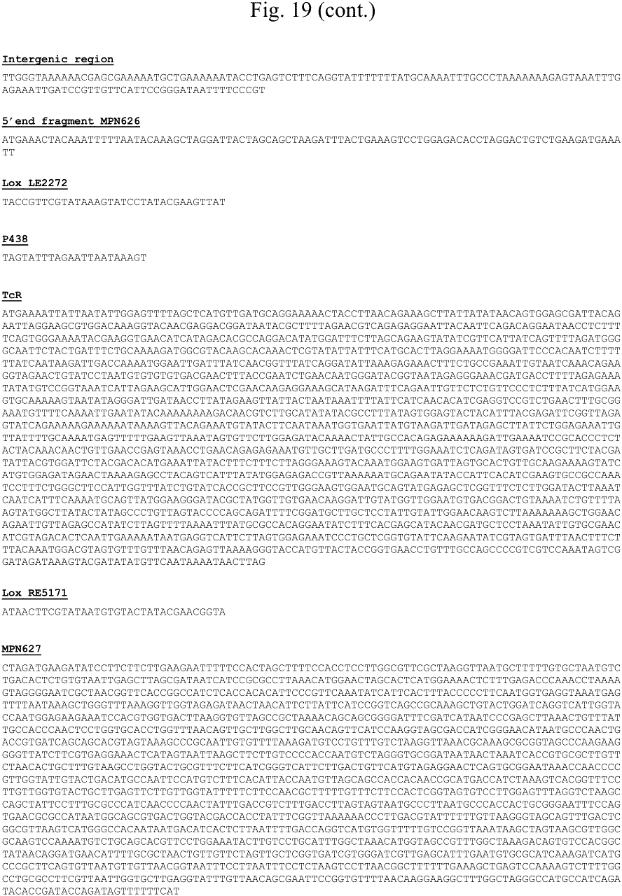

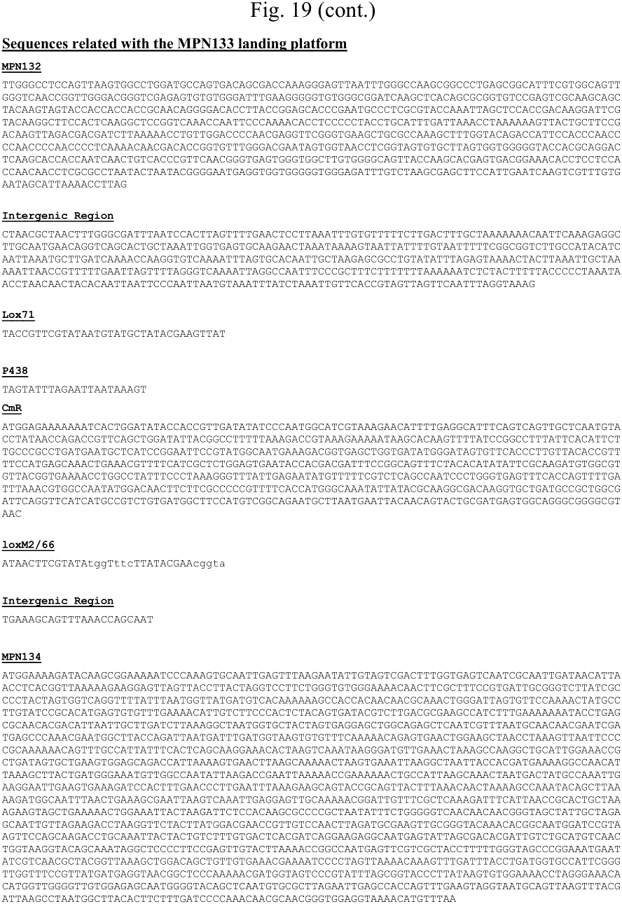

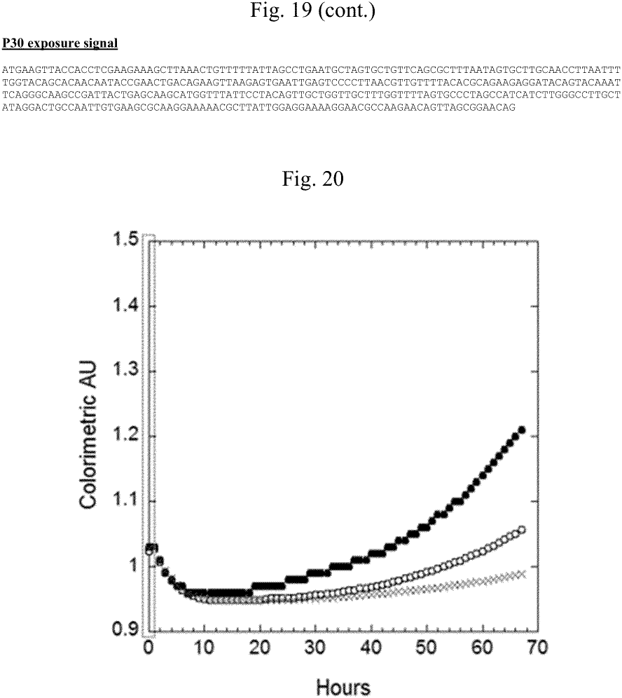

The nucleotide sequences and amino acid sequences of the heterologous expressed proteins are provided in the accompanying sequence listing and in FIGS. 19A-19K.

### 2.2. Peroxide Production and Tissue Lesions

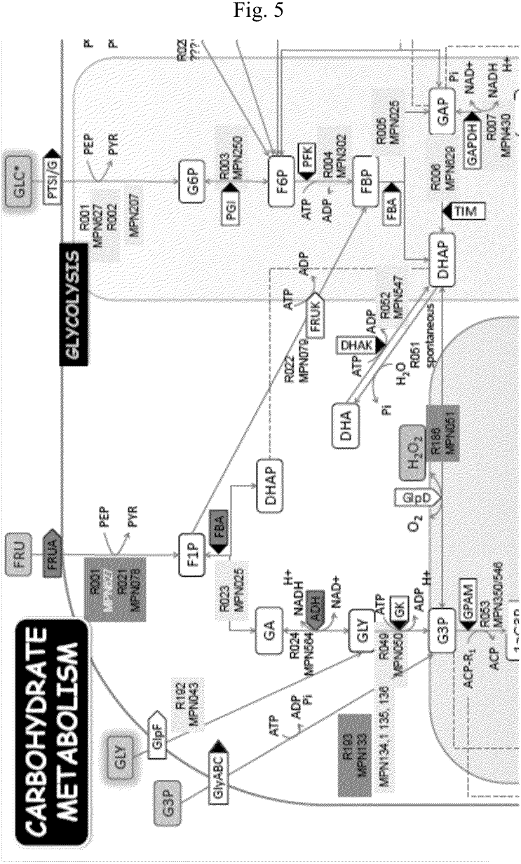

Mycoplasma pneumoniae, a causative agent of respiratory infections, is known to adhere to and colonize the surface of ciliated airway epithelial cells. During this colonization it produces a large amount of hydrogen peroxide as product of glycerol metabolism. This hydrogen peroxide has been demonstrated to be essential for host cell cytotoxicity (Schmidl et al., A Trigger Enzyme in Mycoplasma pneumoniae: Impact of the Glycerophosphodiesterase GlpQ on Virulence and Gene Expression, PLOS pathogens, 2011). Peroxide is produced when either glycerol or phosphatidylcholine is present in the cell culture medium. FIG. 5 depicts the corresponding metabolic pathway is shown. GlpD (encoded by MPN051) catalyzes the oxidation of G3P to dihydroxyacetone phosphate (DHAP) producing hydrogen peroxide. GlpD is a non-essential enzyme (NE) when cells are cultured in presence of glucose but it becomes an essential enzyme when cells are cultured in medium with glycerol or phosphatidylcholine as unique carbon sources. Since in the human lungs one of the main components of the surfactant is phosphatidylcholine, MPN051 is regarded an essential gene in vivo. Thus, removal of MPN051 would lead to a non-viable chassis in the human lungs. To solve this issue, other enzymes in Mycoplasma species have been identified that are able to catalyze the same reaction without using peroxide. One of these enzymes is GpsA from Mycoplasma penetrans. GpsA catalyzes also production of DHAP from G3P in a reversible manner using NADP/NADPH (sn-glycerol 3-phosphate + NAD(P)+ = glycerone phosphate + NAD(P)H + H+).

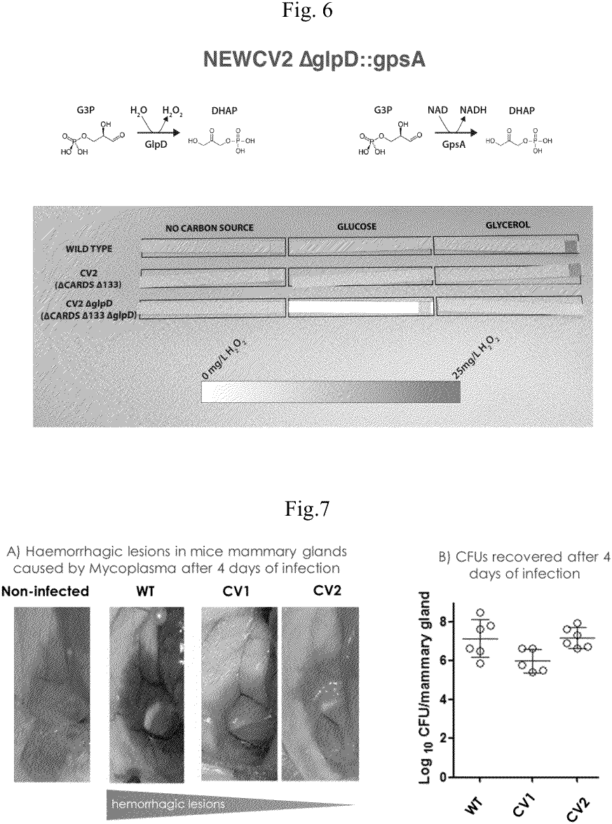

The gpsA gene from M. penetrans was synthesized and configured to be regulated by the constitutive MG438 promoter. This genetic construct was used to replace the MPN051 gene of M. pneumoniae in the CV2 chassis (new chassis CV8). When knocking out the MPN051 gene (Mycoplasma strain CV30) there is no peroxide production and neither the substitution by GpsA produces peroxide (FIG. 6).

The nucleotide sequences and amino acid sequences of the heterologous expressed proteins are provided in the accompanying sequence listing and in FIGS. 19A-19K.

### 2.3. Activation of Innate Immune Response

When characterizing the virulence factors of M. pneumoniae, Kannan and colleagues identified a surfactant protein-A binding (MPN372; CARDS) which is a homologue to the S1 subunit of Bordetella pertussis toxin (Kannan et al., ADP-ribosylating and vacuolating cytotoxin of Mycoplasma pneumoniae represents unique virulence determinant among bacterial pathogens, Proceedings of the National Academy of Sciences of the USA, 2006). The MPN372 protein was found to catalyse adenosine diphosphate ribosylation (ADP-ribosylation) and possess vacuolating function in infected cells (Hardy et al., Analysis of pulmonary inflammation and function in the mouse and baboon after exposure to Mycoplasma pneumoniae CARDS toxin, PLOS one, 2009).

Because of its limited genome size (816 kb), M. pneumoniae lacks the metabolic machinery to generate purines and pyrimidines. Therefore, the intrinsic nuclease activity of Mycoplasmas is essential for their replication and persistence (Razin et al., A partially defined medium for the growth of Mycoplasma. J Gen Microbiol, 1960). Additionally, M. pneumoniae nuclease MPN133 has a cytotoxic effect on mammalian cells, and it uncovers a unique glutamic acid, lysine and serine rich region (EKS region) essential for nuclease binding and internalization but not nuclease activity.

MPN372 is a non-essential gene and was depleted by GP35 mediated recombination (CV1 chassis see Table 7). MPN133 is a non-essential gene and was also deleted by GP35 mediated recombination on the genetic background of the CV1 strain thereby obtaining the CV2 strain (Table 7).

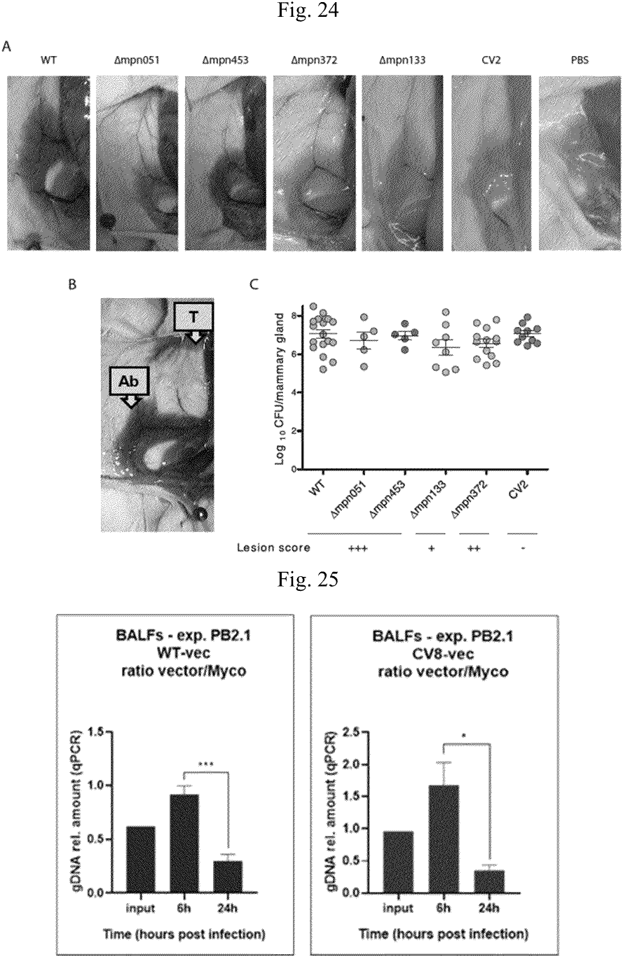

The attenuation degree of CV1 and CV2 was tested in a mice mammary gland model (FIG. 7A). While the WT strain causes a substantial amount of haemorrhagic lesions, it could be observed that this was reduced in the CV1 and CV2 Mycoplasma strains. Additionally, the number of Mycoplasma bacteria present in the tissue after 4 days of infection was quantified by counting colonies formed on agar plates (FIG. 7B). It could be observed that the CFUs recovered for WT, CV1 and CV2 are similar, and that depletions therefore do not affect the maintenance of Mycoplasma in the tissue. Furthermore, the combined deletion of MPN133 and MPN372 invoked a synergistic effect, wherein the resulting double knock out (i.e. CV2) unexpectedly showed no substantial lesions as depicted in FIG. 24. Finally, unexpectedly, these genetically engineered Mycoplasma can be maintained in the lungs of test animals such as mice and are able to produce recombinant proteins when further modified.

### 2.4. Lipoproteins

Inflammatory responses induced by the host’s immunity are one of the main characteristics of M. pneumoniae infection and a major contributor to clinical presentations. Analysis of the cell membrane of Mycoplasma bacteria demonstrated that several lipoprotein surface antigens elicit strong immune responses (Christodoulides et al., The role of lipoproteins in Mycoplasma-mediated immunomodulation, Frontiers in microbiology, 2018) (Table 2). The diacyl glycerol group of lipoproteins activates the inflammasome through interaction with the TRL2 and TRL6 receptors. These proteins also regulate Mycoplasma colonization and translocation across mucosal membranes and facilitate host immune evasion.

TABLE 2

Taken from Christodoulides et al., The role of lipoproteins in Mycoplasma-mediated immunomodulation, Frontiers in microbiology, 2018

| | | | | |
|---|---|---|---|---|
| Lipoprotein/Gene ID | Species of Mycoplasma | Original function | Immunomodulatory effect | References |
| MPN052, MPN162, MPN415, MPN602, MPN611 | M. pneumoniae | F₀F₁ ATP Synthase subunit b | NF-kB activation, Pro-inflammatory diacylated lipoprotein | Shimizu et al., 2008, 2014 |
| MPN597 | M. pneumoniae | F₀F₁ ATP Synthase subunit ε | Induction of inflammation, activation of the autophagy/TLR4-mediated pathway | Shimizu et al., 2014; Shimizu, 2016 |
| MPN611, MPN162 | M. pneumoniae | | Activate NF-κB through TLR pathways | Shimizu et al., 2007 |
| MPN372 | M. pneumoniae | ADP-ribosylating toxin, CARDS toxin | Induces extensive vacuolization and ultimate cell death of mammalian cells, facilitates adhesion to human surfactant protein A (hSP-A), activation of inflammasome | Kannan et al., 2005; Kannan and Baseman, 2006 |
| MPN333 | M. pneumoniae | ABC Transporter | Activation of the autophagy/TLR4-mediated pathway | Shimizu, 2016 |
| MPN142, MPN447, MPN453 Lp44 | M. pneumoniae | Cytoadherence | Activation of inflammasome | Shibata et al., 2000, p. 2; Waldo and Krause. 2006; Spuesens et al., 2011; Balish, 2014; Widjaja et al., 2015 |
| MPN141 | M. pneumoniae | Cytoadherence, P1 adhesin | Induction of pro-inflammatory cytokine production | Waldo and Krause, 2006; Kannan et al., 2011 |

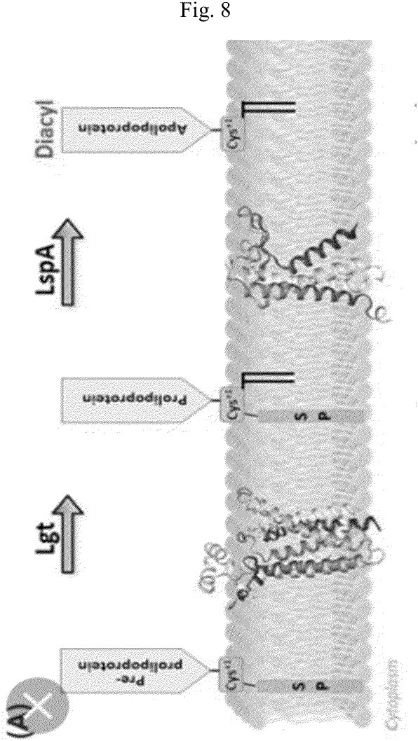

In prokaryotes, membrane lipoproteins are synthesized with a precursor signal peptide, which is cleaved by a specific lipoprotein signal peptidase (signal peptidase II). The peptidase recognises a conserved sequence and cuts upstream of a cysteine residue to which a glyceride-fatty acid lipid is attached (FIG. 8). M. pneumoniae has the two corresponding enzymes, MPN224 (Prolipoprotein diacylglyceryl transferase (Lgt; EC 2.4.99.-)) and MPN293 (LspA; Lipoprotein signal peptidase (EC 3.4.23.36). M. pneumoniae further contains several proteins with the sequence motif recognized by both enzymes (lipobox: [LVI] [ASTVI] [GAS] C) (Table 3).

TABLE 3

List of possible lipoproteins predicted in M. pneumoniae. E: essential (non-dispensable) gene, F: fitness gene, NE: non-essential (dispensable) gene in vitro

| | Gene ID | Essentiality | | Gene ID | Essentiality |
|---|---|---|---|---|---|
| 1 | MPN011 | NE | 34 | MPN592 | NE |
| 2 | MPN052 | F | 35 | MPN639 | NE |
| 3 | MPN054 | NE | 36 | MPN640 | NE |
| 4 | MPN083 | NE | 37 | MPN641 | NE |
| 5 | MPN084 | NE | 38 | MPN642 | NE |
| 6 | MPN097 | NE | 39 | MPN643 | NE |
| 7 | MPN098 | NE | 40 | MPN644 | NE |
| 8 | MPN152 | NE | 41 | MPN645 | NE |
| 9 | MPN199 | E | 42 | MPN646 | NE |
| 10 | MPN200 | NE | 43 | MPN647 | NE |
| 11 | MPN233 | E | 44 | MPN648 | NE |
| 12 | MPN271 | NE | 45 | MPN649 | NE |
| 13 14 | MPN284 MPN288 | NE NE | 46 | MPN650 | NE |
| 15 | MPN363 | NE | 47 | MPN654 | NE |
| 16 | MPN369 | NE | | | |
| 17 | MPN408 | NE | | | |
| 18 | MPN411 | NE | | | |
| 19 | MPN436 | E | | | |
| 20 | MPN439 | NE | | | |
| 21 | MPN442 | NE | | | |
| 22 | MPN444 | E | | | |
| 23 | MPN467 | NE | | | |
| 24 | MPN506 | NE | | | |
| 25 | MPN523 | E | | | |
| 26 | MPN582 | NE | | | |
| 27 | MPN585 | NE | | | |
| 28 | MPN586 | NE | | | |
| 29 | MPN587 | NE | | | |
| 30 | MPN588 | NE | | | |
| 31 | MPN590 | NE | | | |
| 32 | MPN591 | NE | | | |
| 33 | MPN489 | E | | | |

The lipoproteins cited above could activate the TRL2 and/or TLR6 receptor in two possible manners: 1) the proteins can be released from the bacteria due to cell lysis, and/or 2) by release into the medium by the bacteria since these are often proteins of considerable size that are anchored to the membrane only by two short lipid chains. In the first scenario, lipoylation and cleavage of the lipoproteins shown in Table 3 needs to be prevented. In the second scenario only the released proteins need to be targeted. To verify whether the second hypothesis was correct the data provided in the thesis of Bernhard Paetzhold about which proteins are preferentially found in the supernatant of M. pneumoniae cell cultures (Table 4) was analyzed. Several lipoproteins were identified that, despite being acylated at the Cys residue of the consensus signal, are released to the medium (although it cannot be completely discarded that these proteins are proteolytically cleaved on the surface and the released region is detected).

TABLE 4

Proteins found preferentially in the medium where M. pneumoniae is grown (Doctoral thesis available on https://repositori.upf.edu/handle/10230/26935?locale-attribute=en)

| MPN Name | ESSENTIALITY | | Secret. P Score (1) | Ratio MS (2) | PValue J.S. (2) | Ratio MS (3) | PValue J.S. (3) | Ratio MS (4) | PValue J.S. (4) | Ratio MS (5) | PValue J.S. (6) | Confid. Class (6) |
|---|---|---|---|---|---|---|---|---|---|---|---|---|
| 400 | NE | SIGNAL | 0.895 | 77.13 | 0.00 | 43.79 | 0.00 | 22.05 | 0.00 | 9.31 | 0.00 | HC |
| 036 | NE | SIGNAL | 0.667 | 77.44 | 0.00 | 38.00 | 0.00 | 23.24 | 0.00 | 3.90 | 0.00 | HC |
| 592 | NE | Lipoprot | 0.911 | 35.12 | 0.00 | 32.68 | 0.00 | 24.93 | 0.00 | 3.20 | 0.00 | HC |
| 509 | NE | SIGNAL | 0.564 | NA | NA | 25.22 | 0.00 | 17.33 | 0.00 | NA | NA | 2/4 |
| 647 | NE | Lipoprot | 0.935 | 12.35 | 0.00 | 26.75 | 0.00 | 25.22 | 0.00 | 1.17 | 0.00 | HC |
| 084 | NE | Lipoprot | 0.891 | 34.54 | 0.00 | 13.09 | 0.00 | 8.01 | 0.00 | 5.02 | 0.00 | HC |
| 625 | NE | transm. segment | 0.088 | 2.02 | 0.00 | 35.46 | 0.00 | 1.68 | 0.04 | 0.22 | 0.29 | 2/4 |
| 213 | E | SIGNAL | 0.778 | 14.58 | 0.00 | 12.94 | 0.00 | 7.25 | 0.00 | 2.21 | 0.00 | HC |
| 489 | E | Lipoprot | 0.892 | 7.86 | 0.00 | 9.32 | 0.00 | 10.76 | 0.00 | 1.76 | 0.00 | HC |
| 142 | NE | SIGNAL | 0.904 | 12.94 | 0.00 | 7.72 | 0.00 | 2.57 | 0.00 | 6.06 | 0.00 | HC |
| 444 | E | Lipoprot | 0.897 | 1.81 | 0.00 | 18.91 | 0.00 | 1.89 | 0.01 | 1.61 | 0.00 | ⅓ |
| 642 | NE | Lipoprot | 0.841 | 5.01 | 0.00 | 4.49 | 0.00 | 12.18 | 0.00 | 0.56 | 0.00 | HC |
| 398 | NE | Membrane | 0.943 | 2.01 | 0.00 | 3.86 | 0.00 | 10.16 | 0.00 | 0.56 | 0.00 | HC |
| 491 | NE | SIGNAL | 0.934 | 3.16 | 0.00 | 8.02 | 0.00 | 1.51 | 0.09 | 1.14 | 0.00 | ⅓ |
| 083 | NE | Lipoprot | 0.916 | 5.47 | 0.00 | 4.18 | 0.00 | 2.62 | 0.00 | 1.01 | 0.00 | HC |
| 141 | NE | Membrane | 0.934 | 4.00 | 0.00 | 4.46 | 0.00 | 2.00 | 0.01 | 1.39 | 0.00 | ⅓ |

(1): >0.5 = predicted to be secreted, (2): Cell1/Media1 (72 h Experiment 1), (3): Cell1/Sup1 (72 h Experiment 2), (4): Cell4/Media4 (24 h Experiment 1), (5): Cell2/Sup2 (24 h Experiment 2), (6): pValue cutoff= 0.001 for high confidence secreted (HC) in all 4 measurements (HC secreted).

A possible strategy to decrease the response to lipoproteins is to delete those genes which are non-essential and are secreted to the medium (such as MPN083, MPN084, MPN592, MPN642, MPN647) using genome engineering technologies. Alternatively, Lgt and LspA can be inactivated or deleted, resulting in the lipoprotein remaining anchored to the membrane via a transmembrane helical segment. However, this is not possible since both enzymes are essential in Mycoplasma. Another alternative is to replace the transmembrane helical segment of lipoproteins containing the lipobox sequence motif (LVI] [ASTVI] [GAS] C). This occurs in nature as exemplified orthologues of the MPN058 lipoprotein (Table 5). In the case of S. aureus orthologue the Cys essential for cleavage and acylation is lost and the protein is anchored to the membrane via a transmembrane helix.

TABLE 5

Sequence alignment of the N-terminal transmembrane helix of MPN058 gene in M. pneumoniae and its orthologue in S. aureus .The putative helical segment is underlined)
S.aureus MKKIYSFLAGIAAIILVLWGIATH
MPN058 M. pneumoniae MKFKLFLGSSFFGVATLLIACGTG

According to the hypothesis, it should be possible to replace the lipobox sequence motif ([LVI] [ASTVI] [GAS] C) of all essential lipoproteins in M. pneumoniae by a sequence that will not be cleaved and modified at which point MPN224 and MPN293 can be removed from the genome, hereby effectively eliminating lipoylation of proteins. A multiple sequence analysis of Mycoplasma proteins with one transmembrane segment was performed to see if this transmembrane segment is different from the ones modified and cleaved in lipoproteins (Table 6).

TABLE 6

Multiple sequence alignment of essential and fitness lipoproteins and one transmembrane segment at the N-terminal of Mycoplasma proteins. The putative helical segment is underlined
Lipoproteins

| MPN052 | MFGKGLVKKSLLFFSGVSTMAVFLVSCGAT |
|---|---|
| MPN199 | MKFKLFLGSSFFGVATLLIACGTG |
| MPN233 | MKYKTVKSIPLFLLGSIVFTACSTP |
| MPN436 | MKKFLRKPQFWLLTLGGFLSTSVILAACATP |
| MPN444 | MHTFTRKVKWPFMFTAIGLTFGIVAVACAQP |
| MPN456 | MKFQRKYWGLLSTLGVSSAVALSACAAQ |
| MPN489 | MGYKLKRWPLVAFTFTGIGLGWLAACSAL |
| MPN523 | MKKWFSSISKKKVSFSTLLLLGSGIVLSSCSNI |
| One transmembrane helix containing proteins external | |
| MPN213 | MKLSAIISLSVAGTVGTTAVVVPTTITLVNKTHQVEHE |
| MPN405 | MIFSISKRKLICGFSLVALTIAGIVGGVYLVTKNNQQ |
| MPN407 | MKKRTKYRYWLNSLAFFGCGVSVGAFFTLFLMGTQSD |
| MPN505 | MRKKRLLSRISFSSLFLLCGTVLSAYTGIQADLR |
| MPN570 | MKQKIIGLTLAFFVLFLTAVAILFTVKVQRYL |
| One transmembrane helix containing proteins cytoplasmic | |
| MPN244 | MMTVEVFSVIILVLLFLILALTLLFVLLNKRTR |
| MPN268 | MKVLLWIGYVLSFGLLYLYLVKRAKRAALQLNNK |
| MPN269 | SAKLTLESIAKTFAETSIFAILFLIIVILNLGLLVFLAYQYRVYKKK |
| MPN596 | MVVIAALLGSLAVLAFLFYLWYLTIFIIVHKNQQYLEQ |
| MPN674 | MKSLKVALIGSGAVGTSFLYAAMSRGLASEYMV |

In general it can be observed that lipoproteins have shorter helical segments and arguably a higher proportion of G, S, and T when compared with proteins having one transmembrane segment. Proteins exposed to the medium appear to have more positive charged residues N-terminally of the helical segment and the opposite happens with the proteins are faced towards the cytoplasm. To illustrate that a lipoprotein having a transmembrane processed segment could be replaced with a transmembrane segment with no Cys, the MPN489 gene was selected. The gene was deleted and replaced it by a different MPN489 comprising a different N-terminal transmembrane sequence.

The nucleotide and amino acid sequence of rationally designed MPN489 is provided in the accompanying sequence listing and in FIGS. 19A-19K.

### 2.5.Galactocerebrosides

Guillain-Barre syndrome (GBS) is an acute post-infectious immune-mediated polyneuropathy. Although preceding respiratory tract infections with M. pneumoniae have been reported in some cases, the role of M. pneumoniae in the pathogenesis of GBS remains unclear. M. pneumoniae infection is associated with GBS, more frequently in children than adults, and elicits anti-GalactoseCerebroside (GalC) antibodies, of which specifically anti- GalC IgG may contribute to the pathogenesis of GBS (van den Berg et al., Guillain-Barre syndrome: pathogenesis, diagnosis, treatment and prognosis, Nature review neurology, 2014). Antibodies against GalC concomitant to evidence of M. pneumonia infection also have been associated to encephalitis and other nervous disorders (Kusunoki et al., Anti-Gal-C antibodies in GBS subsequent to Mycoplasma infection: evidence of molecular mimicry, Neurology, 2001).

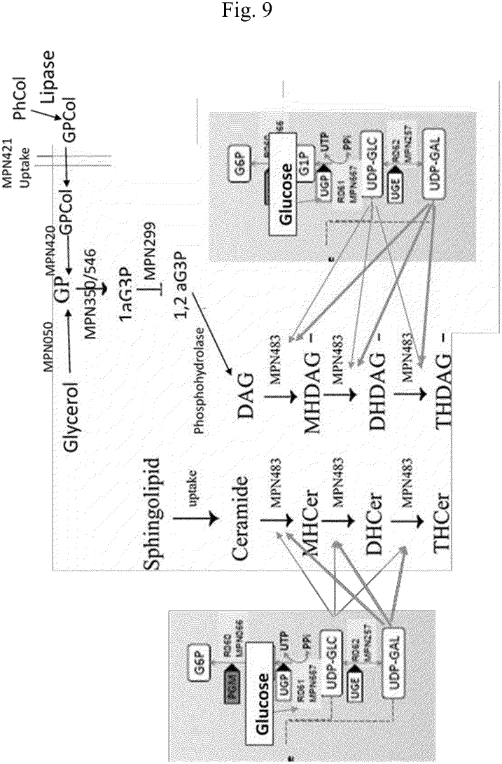

Glycosphingolipids (GSL) are components of most eukaryotic cell plasma membranes. They consist of a ceramide backbone linked to a saccharide polar head group through an O- glyosidic linkage to the C1-hydroxyl of ceramide. Their polar head group consists of a hexose, commonly galactose (galactosylceramide, GalCer) or glucose (glucosylceramide, GlcCer. Galactocerebrosides are typically found in neural tissue, while glucocerebrosides are found in other tissue. A remarkable property of cerebrosides is that their ‘melting point’ is well above physiological body temperature, so that glycolipids have a para-crystalline structure at this temperature. Cerebroside lipids are important for membrane integrity. The ratio between the non-bilayer- forming monoglycosyldiacylglycerols and the bilayer-prone diglycosyldiacylglycerols contributes to regulating the properties of the plasma membrane. M. pneumoniae can synthesize GalC (FIG. 9) by using glucose as a precursor. All the enzymes involved in lipid biosynthesis in M. pneumoniae are either essential genes or genes involved in fitness. Key enzymes in the production of galactocerebrosides are the MPN257 epimerase that produces UDP-galactose out of UDP-glucose and the MPN483 processive glycosil transferase that incorporates preferentially UDP-galactose into β-galactose-diacylglycerol; 50 times over α- glucose-diacylglycerol, and in general prefers UDP-galactose over UDP-glucose regardless of the acceptor.

Enzymes in the MPN483 family all add the sugars in the β-configuration, and a few of the enzymes are also connecting sugars with the β-1,6 linkage. Only galactolipids are synthesized in M. pneumoniae cells, and in vitro assays with solubilized M. pneumoniae cells and solubilized recombinant E. coli cells expressing MPN483 gave a variety of glycolipids. With UDP-Gal as glycosyl donor, DAG and GalβDAG were good acceptors, the latter being preferred to give Galβ1,6GalβDAG. With UDP-Glc as donor, DAG and GalβDAG were acceptors, but not GlcβDAG. Moreover, the main glycolipid synthesized in vivo by recombinant E. coli cells expressing MPN483 was identified as Glcβ1,6GalβDAG. It was concluded that the M. pneumoniae enzyme was mainly a galactosyltransferase, with higher diglycosyltransferase activity on GalβDAG as acceptor, whereas it did not accept GlcβDAG as substrate (Klement et al., A processive lipid glycosyltransferase in the small human pathogen Mycoplasma pneumoniae: involvement in host immune response, Molecular Microbiology, 2007). M. genitalium possess orthologues of the MPN257 (MG_118) and MPN483 (MG517). MG517 sequentially produces monoglycosyl- and diglycosyldiacylglycerols (Andrés et al., Expression and characterization of a Mycoplasma genitalium glycosyltransferase in membrane glycolipid biosynthesis: potential target against mycoplasma infections, The Journal of biological chemistry, 2011). This enzyme in E. coli mainly produces Glcβ1,6GlcβDAG but recognizes also both UDP- glucose and UDP-galactose.

MPN483 is regulated by the presence of phosphatidylglycerol (PG). The amounts of PG included in these assays had a great impact on the different lipids produced, with mainly GalDAG produced at low PG levels and GalGalDAG at high levels, and with total product amounts and proportions changing with increased PG content. MG_517 is activated by dioleoylphosphatidylglycerol (anionic phospholipid), with the k(cat) linearly increasing with dioleoylphosphatidylglycerol concentration (Andrés et al., Expression and characterization of a Mycoplasma genitalium glycosyltransferase in membrane glycolipid biosynthesis: potential target against mycoplasma infections, The Journal of biological chemistry, 2011). Thus it seems that both enzymes are regulated by anionic phospholipids with an increase in the activity to produce di-glycosylated lipids (favors bilayer formation), when the concentration of anionic phospholipids changes.

Based on the above, replacing galactocerebrosides for glucocerebrosides to avoid any autoimmune response due to the exposure outside the nervous system of galactocerebrosides to the immune system is an interesting route to generate improved Mycoplasma strains. In principle, the simplest way to do this is to knock out the galactose epimerase MPN257 that catalyzes the conversion of UDP-glucose into UDP-galactose to force MPN483 to use UDP-glucose to decorate the ceramide making glucocerebrosides, or to knock out enzyme MPN483 preventing formation of galactolipids. Despite MPN483 being a strongly fitness gene as described above, it can be deleted. However, this deletion will likely compromise its growth in vivo. To prevent this possibility, another strategy is to replace the MPN483 enzyme for an equivalent one that only uses UDP-Glucose as a substrate and then knocking out the epimerase MPN257 since other genes downstream of this enzyme are non-essential. As non-limiting example to prove this hypothesis, three different enzymes from three different organisms were selected. It is evident to a skilled person that other additional genes are suitable for this replacement. The first approach was to replace MPN483 by its orthologue from M. genitalium (MG_517) that seems to favor glucolipids over galactolipids (see above). The second approach is to search a related Mycoplasma species that does not have the orthologue of the MPN257 epimerase and therefore if there is an orthologue of MPN483 it should preferentially use UDP-glucose and make glucolipids. For this, M. agalactiae was selected for which essentiality studies have been performed (Montero-Bai et al., DNA research 2019). This orthologue of MPN483 is MAGA_RS00300. A third possibility is to look for a processive glycosil transferase enzyme already characterized and that only uses UDP-glucose as substrate. For this, the utgP enzyme from B. subtilis was selected. This enzyme synthesizes Glucolipids in B. subtilis by processively transferring glucose from UDP-glucose to diacylglycerol (Uniprot entry P54166). Finally, another possibility is to express the alMGS and alDGS enzymes from A. laidlawii that has been shown to recover the phenotype mutant of an UgtP knock out in B. subtilis. In A. laidlawii,α-glucose is linked to DG by alMGS, and then the next glucose is linked to MGlcDG through an α-1,2 bond by alDGS (Matsuoka et al., Suppression of abnormal morphology and extra cytoplasmic function sigma activity in Bacillus subtilis ugtP mutant cells by expression of heterologous glucolipid synthases from Acholeplasma laidlawii, 2016).

The MPN483 gene was replaced by itself as a positive control or by the genes mentioned above to verify whether galactocerebrosides could be removed from M. pneumoniae. Sequences of heterologous genes and proteins are provided in the accompanying sequence listing and FIGS. 19A-19K.

Characterization of the obtained M. pneumoniae strains and their further optimization is detailed in section 5 of the present Examples to emphasize that not only Mycoplasma (e.g.M. pneumoniae) bacteria are envisaged wherein the above described GBS modifications are introduced in addition to the MPN133 and MPN372 modifications, but also Mycoplasma that do not contain MPN133 and/or MPN372 modifications but do nonetheless contain functional modifications such as deletions, insertions and/or substitutions directed to reducing the risk of a recipient to develop GBS. The final collection of genes wherein a functional modification is introduced will evidently depend on the specific application that is envisaged.

### 2.6. Cancer

The inventors have made several genetically modified Mycoplasma strains that comprise a reduced number of genes that encode and optionally express oncoproteins when said Mycoplasma bacterium is expressed in the host organism.

### 2.7. DNA Recombination

The human pathogen M. genitalium employs homologous recombination to generate antigenic diversity in the immunodominant MgpB and MgpC proteins (Iverson-Cabral et al., mgpB and mgpC sequence diversity in Mycoplasma genitalium is generated by segmental reciprocal recombination with repetitive chromosomal sequences, Molecular Microbiology, 2007 and Iverson-Cabral et al., Intrastrain heterogeneity of the mgpB gene in Mycoplasma genitalium is extensive in vitro and in vivo and suggests that variation is generated via recombination with repetitive chromosomal sequences, Infection an immunity, 2006). It is assumed that M. pneumoniae employs a highly similar mechanism. It has been shown in M. genitalium that expression for the alternative sigma factor MG428 (MPN626 in M. pneumoniae) induces expression of the recombination machinery and facilitates recombination (Torres-Puig et al., A novel sigma factor reveals a unique regulon controlling cell-specific recombination in Mycoplasma genitalium, Nucleic acids research, 2015). The MPN626 regulates the same genes involved in recombination than found in M. genitalium and under normal growth conditions it is not expressed (Yus et al., Determination of the Gene Regulatory Network of a Genome-Reduced Bacterium Highlights Alternative Regulation Independent of Transcription Factors, 2019, Cell Systems). However, it is possible that in vivo under certain conditions, the gene could be expressed and recombination induced. To remove this possibility the MPN626 gene was depleted and replaced by a landing platform flanked by Cre-Lox sites to insert DNA constructs (CV8_L2). The mutant grows similarly to CV8 (see Table 9).

### 2.8. Increasing Cell Doubling Time

M. pneumoniae divides every 8-10 hours under optimal in vitro culture conditions. Thus, culturing the bacteria in a fermenter takes a substantial amount of time which is accompanied by a considerable reagents cost. Thus, having a M. pneumoniae strain that could grow faster could be very useful for biotechnology purposes. It is known that duplicating the number of rRNA operons from one to two can increase growth rate by ~20%. In addition, other genes have been identified that, upon overexpression, could increase growth rate (Determinants of Growth Rate in Genome-reduced Bacteria, Carolina Gallo Lopez, Universitat Pompeu Fabra, 2018). Similarly it was demonstrated that ribosomes and genes encoding for proteins involved in translation seem to be related to the speed of cell division in different Mycoplasma species. Thus doubling the rRNA operon as well as increasing the expression of ribosomal proteins by modifying their promoters is a promising route to obtain Mycoplasma strains having faster cell division rates.

### 2.9. Overview of Genetically Modified Mycoplasma Strains

A compendium of preferred Mycoplasma strains that are of particular interest are summarized in Table 7 below.

TABLE 7

Genetically modified Mycoplasma strains of particular interest. In the column that displays the deleted or inactivated genes “/” indicative for independent genes while “-” indicates a continuous region from one gene to the other

| | | | |
|---|---|---|---|
| Strain | Strain background | Genes deleted or inactivated | Heterologous genes optionally expressed |
| WT M129 | - | | |
| CV0 | | | gp35 |
| CV1 | WT | ΔMPN372 | gp35 |
| CV2 | CV1 | ΔMPN372/ΔMPN133 | gp35 |
| CV3 | CV2 | ΔMPN372/ΔMPN133/MPN142 | gp35 |
| CV4 | CV2 | ΔMPN372/ΔMPN133/ΔMPN491 | gp35 |
| CV5 | CV4 | ΔMPN372/ΔMPn133/ΔMPN491/ΔMPN400 | gp35 |
| CV6 | CV1 | ΔMPN372/MPN294 | gp35 |
| CV7 | CV2 | ΔMPN372/MPN133 | MG_186;gp35 |
| CV8 | CV2 | ΔMPN372/ΔMPN051/ΔMPN133 | gpsA;gp35 |
| CV9 | CV2 | ΔMPN372/ΔMPN133 | Vsa;gp35 |
| CV10 | WT | | Vsa |
| CV11 | CV4 | ΔMPN372/MPN133/MPN491 | Vsa;gp35 |
| CV12 | CV8 | ΔMPN372/ΔMPN051/ΔMPN133 | gpsA and Vsa;gp35 |
| CV13 | CV3 | ΔMPN372/ΔMPM133/ΔMPN142 | Vsa;gp35 |
| CV14 | CV8 | ΔMPN372/MPN051/MPN133 | gpsA and P97; gp35 |
| CV15 | WT | | P97 |
| CV16 | CV0 | ΔMPN453 | gp35 |
| CV17 | CV2 | ΔMPN372/ΔMPN133/ΔMPN400 | gp35 |
| CV18 | WT | | rRNA |
| CV19 | CV2 | ΔMPN372/ΔMPN133/ΔMPN453 | gp35 |
| CV20 | CV2 | ΔMPN372/ΔMPN133/ΔMPN051 | gp35 |
| CV23 | CV20 | ΔMPN372/ΔMPN133/ΔMPN051/ΔMPN400 | gp35 |
| CV24 | CV8 | ΔMPN372/ΔMPN133/ΔMPN051/ΔMPN415 | gpsA;gp35 |
| CV25 | CV20 | ΔMPN372/ΔMPN133/ΔMPN051/ΔMPN415 | gp35 |
| CV29 | CV8 | ΔMPN372/ΔMPN133/ΔMPN051/ΔMPN400 | gpsA;gp35 |
| CV30 | WT | TnΔMPN051 | |
| CV31 | CV0 | ΔMPN133 | gp35 |
| CV32 | WT | --- | gp35;pTETCre |
| CV33 | CV32 | ΔMPN483 | gp35;pTETCre |
| CV34 | CV32 | ΔMPN372/ΔMPN133/ΔMPN483 | gp35;pTETCre;mpn483 |
| CV35 | CV32 | ΔMPN372/ΔMPN133/ΔMPN483 | gp35;pTETCre;GT subtilis |
| CV36 | CV32 | ΔMPN372/ΔMPN133/ΔMPN483 | gp35;pTETCre;GT agalactiae |
| CV37 | CV32 | ΔMPN372/ΔMPN133/ΔMPN483 | gp35;pTETCre;GT genitalium |
| CV38 | CV32 | ΔMPN490-ΔMPN505 (15kb) | gp35;pTETCre |
| CV39 | CV32 | ΔMPN490-ΔMPN506 (20kb) | gp35;pTETCre |
| CV40 | CV32 | ΔMPN490-ΔMPN509 (25kb) | gp35;pTETCre |
| CV41 | CV32 | ΔMPN592 | gp35;pTETCre |
| CV42 | CV32 | ΔMPN083-MPN084 | gp35;pTETCre |
| CV43 | CV32 | ΔMPN640-MPN647 | gp35;pTETCre |
| CV8-L2 | CV8 | ΔMPN626 | gpsA;gp35;landing platform |
| CV44 | WT | ΔMPN257/ΔMPN483 | gp35 |
| CV45 | CV2 | ΔMPN372/ΔMPN133/ΔMPN257/ΔMPN483 | gp35 |

### 2.10. Landing Platforms for Cloning

To facilitate cloning and ensure a similar genomic environment of all gene fragments introduced in the genome of M. pneumoniae, certain genomic elements are introduced into the bacterium which are herein further referred to as “landing platforms”. These landing platforms are DNA fragments (i.e. DNA sequences) comprising an antibiotic resistance gene flanked by non-compatible Cre-Lox sites. Thus, when a vector containing the Cre recombinase and the desired gene fragment flanked by the corresponding lox sites is transformed, the antibiotic gene cassette is replaced by the desired DNA piece. The sequences used for generating two such landing platforms in Mycoplasma, one located at the MPN626 gene and the other at the MPN133 gene are given in the accompanying sequence listing and in FIGS. 19A-19K.

### 3 Characterization Assays for Modified Mycoplasma Strains

### 3.1. Murine Mastitis Model

### 3.1.A. Animal Infection

Female CD1 mice (Charles River International) of 7 weeks were accommodated in the animal facilities of the Universidad Pública de Navarra (UPNA; registration code ES/31-2016-000002-CR-SU-US), with water and food ad libitum. Mouse handling and procedures were performed in compliance with the current European and national regulations, following the FELASA and ARRIVE welfare guidelines, with the supervision of the UPNA’s Comité de Ética, experimentación Animal y Bioseguridad (CEEAB) and with approval of the competent authority (Gobierno de Navarra).

CD1 lactating mice were used 10-12 days post parturition, weighing on average ±40 g. The infection model implemented as described in the protocol of Brouillette and collaborators (Brouillette et al., Mouse mastitis model of infection for antimicrobial compound efficacy studies against intracellular and extracellular forms of Staphylococcus aureus. Veterinary Microbiology, 2004). Prior to bacterial inoculation each animal was anesthetized by intraperitoneal administration of ketamine (100 mg/kg of body weight; Imalgene; Merial Laboratorios, S.A.) and xylacine (10 mg/kg; Rompun; Bayer Health Care). The pups were removed and a 100 µL syringe with a 33-gauge blunt needle (Hamilton) was used to inoculate the R4 (right) abdominal mammary gland. 100 µl suspended ±1×10⁹ CFU/ml Mycoplasma or suspended protein was administered through the orifice of the mammary gland (1×10⁸ CFU/mouse).

### 3.1.B. Dose-Response Mastitis Model

Five CD-1 lactating mice were used for each inoculum condition. The doses (CFU/mouse) tested were 5 × 10⁴ and 1.3×10⁵ and 1× 10⁶ for Mycoplasma WT and S. aureus 15981, respectively. At day 1 (S. aureus) or day 4 (Mycoplasma) post-inoculation (PI), animals were sacrificed and mammary glands were aseptically obtained, weighed and homogenised in phosphate buffered saline (PBS). For S. aureus, the kidneys were pooled, weighed and homogenized. Bacterial counts were determined by plating logarithmic dilutions of the samples on Tryptic Soy agar (TSA) or Hayflick (BD) plates supplemented with Ampicillin (VWR) 100 mg/ml (H-Amp agar), for S. aureus or Mycoplasma, respectively.

### 3.1.C. Growth Curves

Five CD-1 lactating mice were used for each time condition. The doses (CFU/mouse) tested for Mycoplasma WT were 1× 10⁵ or 1×10⁶, and 5×10⁴ for S. aureus 15981. At days 1, 4 and 8 post-infection, animals were sacrificed and mammary glands were aseptically obtained, weighed and homogenized in PBS. Bacterial counts were assessed as described above. In addition, differences in growth of the WT vs modified Mycoplasma strains was assessed. Thus, the modified Mycoplasma strains (e.g. the CV2 strain) at a dose (CFU/mouse) of 1×10⁵ was tested both at day 1 and 8.

### 3.1. D. Screening of Virulence of Selected Strains

Five CD-1 lactating mice were used and the experiment was done twice to analyse the virulence of the strains in the mastitis mice model. Animals were anesthetized, pups removed and a volume of 100 µL of bacterial suspension was administered through the orifice of the mammary as outlined in detail above. 4 days post infection animals were sacrificed and mammary glands obtained and excised for bacterial counts (as explained above) and interleukin (IL) detection (see section 4).

### 3.1.E. Histopathological Analysis

Histopathological analyses and lesion scorings were carried out in portions of mammary gland that were fixed in 10% formaldehyde (Sigma) solution. Animals not subjected to infection (PBS administration) were used as control condition. Five trans axial slices of tissues obtained every 2 to 3 mm were embedded in paraffin. 4- to 6-µm sections were stained with Haematoxylin Eosin by standard procedures and examined for histological features including adenomer lesions, secretory duct lesions, or inflammatory lesions. Lesion were scored using a scoring scale of from 0 to 4 (0, absent to very low; 1, mild; 2, moderate; 3, strong; 4, very strong).

### 3.1.F. Whole Mammary Gland RNA Extraction and Real-Time Quantitative PCR (RT-qPCR) Analysis

The right mammary gland was homogenized using Ultra-Turrax (IKA) and was followed by total RNA isolation using an RNeasy® Mini Kit (Qiagen) according to the manufacturer’s instructions. RNA concentration was measured spectrophotometrically using a Nanodrop One device (Thermo-Scientific) and RNA integrity was confirmed by agarose gel electrophoresis. RNA samples with absorbance at 260:280 nm ratios of 1.8-2.1 were retained for real-time reverse-transcriptase PCR. Complementary DNA (cDNA) from whole mammary gland cells was synthesized from 1 µg total RNA using SuperScript II Reverse Transcriptase reagents (Invitrogen). PCR amplification was performed using SYBR Premix Ex Taq II (Tli RNaseH Plus) (Takara) and fluorescence was analyzed with AriaMx Real-Time PCR System (Agilent Technologies). The comparative threshold cycle (Ct) method was used to obtain relative quantities of mRNAs that were normalized using GAPDH as housekeeping gene. Primers pairs for TNF-a, KC (IL-8), INF-y, IL-1b, IL-4, IL-6, IL-17, IL-18, IL-22, and GapdH mRNA detection are shown (Table 8).

TABLE 8

qPCR primers used for detection of Interleukin expression and other immunomodulators

| Primer | ID | Sequence (5′-3′) | Reference |
|---|---|---|---|
| rgapdH_qRT-PCR_F | 135 | CCCACTAACATCAAATGGGG | Regueiro et al., 2011 |
| rgapdH_qRT-PCR_R | 136 | CCTTCCACAATGCCAAAGTT | Regueiro et al., 2011 |
| rtnf-α_qRT-PCR_F | 137 | AGGCACTCCCCCAAAAGA | Euba et al., 2017 |
| rtnf-α_qRT-PCR_R | 138 | GCTCCTCCACTTGGTGGTTT | Euba et al., 2017 |
| rkc_qRT-PCR_F | 139 | GACAGACTGCTCTGATGGCA | Euba et al., 2017 |
| rkc_qRT-PCR_R | 140 | TGCACTTCTTTTCGCACAAC | Euba et al., 2017 |
| rinfy_qRT-PCR_F_4 | 207 | CTGGCAAAAGGATGGTGAC | This study |
| rinfy_qRT-PCR_R_4 | 208 | GCTGATGGCCTGATTGTCTT | This study |
| rmcp-1_qRT-PCR_F2 | 233 | CTTCTGGGCCTGCTGTTCA | Kei Goto et al., 2016 |
| rmcp-1_qRT-PCR_R2 | 234 | CCAGCCTACTCATTGGGATCA | Kei Goto et al., 2016 |
| rmip-1a_qRT-PCR_F | 228 | CCCAGCCAGGTGTCATTTTCC | Wang et al., 2019 |
| rmip-1a_qRT-PCR_R | 227 | GCATTCAGTTCCAGGTCAGTG | Wang et al., 2019 |
| rtlr2_qRT-PCR_F | 230 | CTCCTGAAGCTGTTGCGTTAC | Wang et al., 2019 |
| rtlr2_qRT-PCR_R | 229 | GCTCCCTTACAGGCTGAGTTC | Wang et al., 2019 |
| ril-1b_qRT-PCR_F | 141 | GCTGCTTCCAAACCTTTGAC | Shen et al., 2018 |
| ril-1b_qRT-PCR_R | 142 | AGCTTCTCCACAGCCACAAT | Shen et al., 2018 |
| ril-4_qRT-PCR_F_4 | 201 | GCAACGAAGAACACCACAGA | This study |
| ril-4_qRT-PCR_R_4 | 202 | ATCGAAAAGCCCGAAAGAGT | This study |
| ril-6_qRT-PCR_F | 144 | TGATGCACTTGCAGAAAACA | Leili et al., 2019 |
| ril-6_qRT-PCR_R | 145 | GGTCTTGGTCCTTAGCCACTC | Leili et al., 2019 |
| ril-12p35_qRT-PCR_F | 221 | CCAAGGTCAGCGTTCCAACA | Happel et al., 2005 |
| ril-12p35_qRT-PCR_R | 222 | AGAGGAGGTAGCGTGATTGACA | Happel et al., 2005 |
| ril-12p40_qRT-PCR_F | 223 | CAGCACCAGCTTCTTCATCAG | Happel et al., 2005 |
| ril-12p40_qRT-PCR_R | 224 | TCTTCAAAGGCTTCATCTGCAA | Happel et al., 2005 |
| ril-18_qRT-PCR_F | 149 | ACTGTACAACCGCAGTAATACG | Brieland et al., 2000 |
| ril-18_qRT-PCR_R | 150 | AGTGAACATTACAGATTTATCCC | Brieland et al., 2000 |
| ril-23_qRT-PCR_F2 | 231 | TGCTGGATTGCAGAGCAGTAA | Kei Goto et al., 2016 |
| ril-23_qRT-PCR_R2 | 232 | GCATGCAGAGATTCCGAGAGA | Kei Goto et al., 2016 |

### 3.1.G. Preventive Treatment Mastitis Model

To evaluate the efficacy of a preventive mastitis model, CD1 female mice were used as explained above. Once animals were anesthetised, a 100 µl of the treatment was intramammary inoculated. Thus, a100 µl suspension of Mycoplasma WTDBLys P3 or CV2DBLysP3 with ≈1× 10⁹ CFU/ml was inoculated (1× 10⁸CFU/mouse), recombinant lysostaphin (25 µg/mouse) or PBS (non-treated) with a blunt end gauge (Hamilton). Mice were challenged with 100 µl of S. aureus 15981 suspension with ≈5×10⁵ UFC/ml (5 ×10⁴ CFU/mouse) 30 min later. The dams were euthanized 18 h post-infusion, mammary glands were aseptically dissected and portions of the glands were used to determine bacterial loads of Mycoplasma and S. aureus.

### 3.1.H. Statistical Analyses

In each analysis, a p value of <0.05 was considered statistically significant. Analyses were performed using GraphPad Prism software, version 5.01 (GraphPad Software) statistical package.

### 3.2. Murine Lung Infection Model.

### 3.2.A. Mycoplasma Pneumoniae Mouse Healthy Lung Infection

CD1, C57Bl/6, Balb/C male or female mice (18-22 g) aged 4-6 weeks were purchased from Charles River Laboratories (France), housed under pathogen-free conditions at the Institute of Agrobiotechnology facilities (registration number ES/31-2016-000002-CR-SU-US), and used at 25-28 g. Animal handling and procedures were in accordance with the current European (Directive 86/609/EEC) and National (Real Decreto 53/2013) legislations, following the FELASA and ARRIVE guidelines, and with the approval of the Universidad Pública de Navarra (UPNa) Animal Experimentation Committee (Comité de Ética, Experimentatión Animal y Bioseguridad) and the local Government authorization. Mycoplasma pneumoniae strains were used for intratracheal infection by administration of 100 µl of bacterial suspension containing ~0.5-1×10⁸ CFU/ml (~0.5-1×10⁷ CFU/mouse), in mice previously anesthetized with isoflurane 2% (ISOFLO, Covegan). When necessary, CD1 mice were infected with ~1×10¹⁰ CFU/ml (~1×10⁹ CFU/mouse). Infections were performed in groups of at least five mice per strain and time point (n ≥ 5). At 2 or 4 dpi, mice were euthanized using cervical dislocation, before removal of the lungs. The left lung was individually weighed in sterile bags (VWR) and homogenized 1: 10 (wt/vol) in PBS. Each homogenate was serially 10-fold diluted in PBS and plated in triplicate on Hayflick-Amp100 agar to determine the number of CFU per lung and for the essentiality assay. The right lung was fixed in 10% neutral buffered formalin for histological analysis or stored at -80° C. for RNA extraction. Experiments are summarized in table 9.

TABLE 9

Summary of Mycoplasma pneumoniae mouse healthy lung infection experiments. ID, experiment identification

| Strain | Mice strain | | | | | | | | |
|---|---|---|---|---|---|---|---|---|---|
| | CD1 | | | C57B⅙ | | | Balb/C | | |
| | 2 dpi | 4 dpi | ID | 2 dpi | 4 dpi | ID | 2 dpi | 4 dpi | ID |
| WT | X | X | MR20/21R MR22 (1) MR31 MR32 MR32.2 | | X | MR26² | X | X | MR3 3 |
| | X | | MR30¹ | | | | | | |
| WTV ar | X | X | MR20/21R MR22 (1) | | | | | | |
| CV2 | X | X | MR20/21R MR22 (2) MR27 MR31 MR32 | | X | MR26² | X | X | MR3 3 |
| CV2Var | X | X | MR20/21R MR22 (2) | | | | | | |
| CV8 (CV2 glpD::gpsA) | X | X | MR20/21R MR22 (2) MR27 | | | | X | X | MR3 3 |
| CV8Var (CV2Var glpD::gpsA) | X | X | MR20/21R MR22 (2) | | | | | | |
| WTAp30 | X | X | MR32 MR32.2 | | | | | | |
| WTAMPN151(Tn glpD) | X | X | MR32 MR32.2 | | | | | | |

¹Mice were infected with ~1×10¹⁰ CFU/ml (~1×10⁹ CFU/mouse)
²Fonseca-Aten et al., Mycoplasma pneumoniae Induces Host-Dependent Pulmonary Inflammation and Airway Obstruction in Mice, American Journal of Respiratory Cell and Molecular Biology, 2004.

### 3.2.B. Mycoplasma Pneumoniae Mouse Emphysema Lung Infection

Emphysema was induced by intratracheal administration of porcine pancreatic elastase (PPE) (EPC, Elastin Products Company). First, 10 mg containing 1,350 elastase units (U) were suspended in 10 ml physiological serum (SSF) to generate a stock solution (1 mg/ml, i.e. 135 U/ml). To induce emphysema, one 90 µl dose containing 6 elastase U/mouse was administered 17 days before infection (Artaechevarria et al., Longitudinal study of a mouse model of chronic pulmonary inflammation using breath hold gated micro-CT, European radiology, 2010; Artaechevarria et al., Evaluation of micro-CT for emphysema assessment in mice: comparison with non-radiological techniques, 2011; Fernandez-Calvet et al., Modulation of Haemophilus influenzae interaction with hydrophobic molecules by the VacJ/MlaA lipoprotein impacts strongly on its interplay with the airways, Scientific reports, 2018). When necessary, mice were randomly divided into three groups (n= 5): (i) control mice with normal lung function; (ii) mice with lung emphysema; (iii) elastase vehicle solution. Different M. pneumoniae strains were used for intratracheal infection by administration of 100 µl of bacterial suspension containing ~0.5-1×10⁸ CFU/ml (~0.5-1×10⁷ CFU/mouse), in mice previously anesthetized with isoflurane 2% (ISOFLO, Covegan). The left lung was individually weighed in sterile bags (VWR) and homogenized 1:10 (wt/vol) in PBS. Each homogenate was serially 10-fold diluted in PBS and plated in triplicate on Hayflick-Amp100 agar to determine the number of CFU per lung and for the essentiality assay. The right lung was fixed in 10% neutral buffered formalin for histological analysis or stored -80° C. for RNA extraction.

The histological analysis confirmed the presence of emphysema in the PPE-treated animals (all, p<0.0001). At 2 dpi, interstitial pneumonia accompanied of peribronchial infiltration was identified in the PPE/CV2+ lungs in comparison with the PPE/CV2- ones (both, p<0.05). Otherwise at 4 dpi, despite the fact that the CV2 strain presented higher counts in the PPE-treated lungs than in the vehicle ones, histological findings were comparable between PPE/CV2+ and PPE/CV2-groups. The CV2 chassis infected lungs presented the same inflammatory profile as those observed in the non-infected ones, independently of the lung status, at all-time points tested.

### 3.2.C. Infection Essentiality Assay

The remaining volume of lung homogenates of the infected animals were passed through a 0.45 µm filter and 100 µl was inoculated into a T25 flask with 5 ml Hayflick-Amp100-Tet2. The solution was cultured until the broth turned an orange hue (±72 h). At this point, the supernatant was decanted, 3 ml of PBS was added to the flask and a cell scraper was used to harvest the adherent Mycoplasmas from the bottom of the flask. The total volume was distributed in aliquots of 1 ml and stored at -20° C. until further use. Aliquots were thawed at room temperature, and after centrifugation (13000 rpm, 5 minutes) the obtained pellet was used for genomic extraction using a DNeasy® UltraClean Microbial Kit (Qiagen) according to the manufacturer’s instructions. DNA concentration was measured spectrophotometrically using a Nanodrop One device (Thermo-Scientific) and sample DNA integrity was confirmed by 1% agarose gel electrophoresis. The genetic background was verified by PCR with the following primers pairs: qPCR_P1_F (5′ACGATGATTACAGGCGGTTC) + qPCR_Pl_R (5′ GTTGGTGGCCTCTTGTTGAT); MPN142_F (5′TCCCAGCAAGTGTGAACCC) + MPN142 R (5′ GTTTTCGCTCATCAGGTCG); CARDS_F (5′ CAAAAACAAGGACCCCGTCG) + CARDS_R (5′ CATTCAACCCAAACCAAAGC); MPN133_F (5′ATTTGTCTAAGCGAGCTTCC) + MPN133_R (5′TTAATCTGGTAAGCCATTCG).

### 3.2.D. Whole Lung RNA Extraction and Real-time Quantitative PCR (RT-qPCR) Analysis

The right lung was homogenized using Ultra-Turrax (IKA) and total RNA was isolated using an RNeasy® Mini Kit (Qiagen), according to the manufacturer’s instructions. RNA concentration was measured spectrophotometrically using a Nanodrop One device (Thermo-Scientific) and sample RNA integrity was confirmed by 1% agarose gel electrophoresis. RNA samples with 260:280 nm absorbance ratios of 1.8 to 2.1 were used for real-time reverse-transcriptase PCR (RT). Complementary DNA (cDNA) from whole lung cells was synthesized from 1 µg total RNA using SuperScript II Reverse Transcriptase reagents (Invitrogen). PCR amplification was performed using SYBR Premix Ex Taq II (Tli RNaseH Plus) (Takara) wherein fluorescence was analysed with the AriaMx Real-Time PCR System (Agilent Technologies). The comparative threshold cycle (Ct) method was used to obtain relative quantities of mRNAs that were normalized using GAPDH as suitable housekeeping gene (Livak et al., Analysis of relative gene expression data using real-time quantitative PCR and the 2(-Delta Delta C(T)) Method, Methods, 2001). Primers pairs for RNF-α, KC (IL-8), IFN-γ, MCP-1, MIP-1a, TLR2, IL-1b, IL-4, IL-6, IL-12p35, IL-12p40, IL-18, IL-23 and GAPDH mRNA detection are shown in Table 8.

### 3.2.E. Statistical Analysis

In each analysis, p values below 0.05 were considered statistically significant. Analyses were performed using Prism software, version 8 for Mac (GraphPad Software) statistical package and are indicated in each figure legend.

### 4. Further Characterization of Modified Mycoplasma Strains

After generation, numerous parameters were experimentally assessed to further characterize the obtained genetically modified Mycoplasma strains. These parameters include growth rate, peroxide production, pathogenicity, infectivity, and inflammatory response induced in a host organism.

### 4.1. Growth Curves

Cell doubling times were calculated using two different methods. In a first approach, qPCR arbitrary unit values were log2 transformed prior to linear fitting after removal of the early and late points of the growth curve. In the second approach, exponential fitting of the data was done. Additionally, the final biomass was calculated by quantifying total protein amount. Further, an indirect estimation of the biomass increase was derived by determining the maximum difference of the log2 qPCR values. Results are summarized in Table 10.

TABLE 10

Growth curve parameters of different Mycoplasma strains

| | Cell division rate Hours Linear fitting | Cell division rate Hours exponential fitting | Final log2 biomass ugr | log2 qPCR (diff min and max values) | |
|---|---|---|---|---|---|
| WT | 7.6687 | 7.0180 | 23.1085 | 5.8000 | WT |
| CV0 | 8.8984 | 7.7440 | 22.7725 | 5.2000 | WT |
| CV1 | 8.2102 | 7.7510 | 22.3880 | 4.9000 | CV0 Δ372 |
| CV2 | 8.5859 | 8.8510 | 21.6694 | 4.7000 | CV1 Δ133 |
| CV3 | 7.6359 | 7.0990 | 22.1964 | 6.5000 | CV2 Δ142 |
| CV4 | 7.1999 | 6.9960 | 22.6862 | 6.2000 | CV2 Δ491 |
| CV5 | 8.1987 | 7.3710 | 22.3016 | 5.5000 | CV4Δ400 |
| CV6 | 7.2902 | 8.6650 | 22.6896 | 6.3000 | CV1Δ294 |
| CV7 | 6.6168 | 8.9520 | 22.6157 | 6.9000 | CV1Δ133 |
| CV8 | 6.5595 | 6.6340 | 22.8673 | 6.6000 | CV2Δ051 |
| CV8_L2 | 6.7847 | 5.9440 | 22.4706 | 6.4000 | CV8Δ626 |
| CV14 | 8.5948 | 7.9690 | 21.8979 | 5.3000 | CV8_P97 |
| CV15 | 6.3052 | 7.8510 | 22.1773 | 5.8000 | WT_P97 |
| CV16 | 8.2399 | 8.3810 | 23.7627 | 6.1000 | CV0 Δ453 |
| CV17 | 7.0711 | 8.9520 | 21.9370 | 6.3000 | CV2 Δ400 |

### 4.1. Proteomics

Proteomics experiments have shown that the genetically modified Mycoplasma bacteria as described herein contain a modified proteome expression signature that contribute to improved characteristics when said Mycoplasma bacterium is introduced in the host organism. The proteomics experiments are conducted according to the following standard protocol:

For the proteome samples of different mutant strains, Mycoplasma strains were grown at an exponential phase of growth. Medium was removed and cells were washed twice with PBS. Total protein extracts were obtained by lysing the cells with 200 µl of lysis buffer (4% SDS, 0.1 M DTT and 0.1 M Hepes). The total protein extracts of two biological replicates were analyzed by LC/MS/MS. Briefly, samples were dissolved in 6 M urea, reduced with 10 mM dithiothreitol (37° C., 60 min), and alkylated with 20 mM iodoacetamide (25° C., 30 min). Samples were diluted 6-fold with 0.2 M NH₄HCO₃ before being digested at 37° C. overnight with trypsin (at a protein:enzyme ratio of 10:1). Peptides generated upon digestion were desalted, evaporated to dryness and dissolved in 0.1% formic acid. An aliquot of 2.5 µl of each fraction (amounts ranging from 0.17 to 4 µg) was run on an LTQ-Orbitrap Velos (Thermofisher) fitted with a nanospray source (Thermofisher) after nanoLC separation in an EasyLC system (Proxeon). Peptides were separated in a reverse phase column, 75 µm × 250 mm (Nikkyo Technos Co., Ltd.) with a gradient of 5 to 35% acetonitrile in 0.1% formic acid for 60 min at a flow rate of 0.3 mL/min. The Orbitrap Velos was operated in positive ion mode with the nanospray voltage set at 2.2 kV and its source temperature at 325° C. In addition, 20 µg of the total extract was digested and desalted and 1 µg of the resulting peptides analyzed on an Orbitrap Velos Pro in identical conditions as the fractions albeit with a longer gradient (120 min). A total of three biological replicates were done, as well as two technical replicates for each strain in two independent experiments. The spectra were assigned to peptides by using Mascot and a customized database comprising all ORFs longer than 19 amino acids. Only the areas of the three best unique peptides were used to estimate the protein amounts.

From these experiments, one can observe the changes in protein expression of endogenous Mycoplasma strain proteins upon gene deletion.

### 4.2 Metabolomics and Lipidomics

In particular for the genetically modified Mycoplasma strains that are obtained with the goal of reducing or eliminating the risk to develop Guillain-Barre syndrome, metabolomics experiments allow for a thorough characterization of changes in galactocerebroside composition. Lipids were analyzed as described before (Simbari et al., Plasmalogen enrichment in exosomes secreted by a nematode parasite versus those derived from its mouse host: implications for exosome stability and biology. Journal of Extracellular Vesicles 2016; and Barbacini et al., Regulation of serum sphingolipids in Andean Children born and living at high altitude (3775 m). International Journal of Molecular Sciences, 2019) with minor modifications as described below.

Phospholipids and neutral lipids: A total of 750 µl of a methanol-chloroform (1:2, vol/vol) solution containing internal standards (16:0 D31_18:1 phosphocholine, 16:0 D31_18:1 phosphoethanolamine, 16:0 D31-18:1 phosphoserine, 17:0 lyso-phosphocholine, 17:1 lyso-phosphoethanolamine, 17:1 lyso-phosphoserine, 17:0 D5_17:0 diacylglycerol, 17:0/17:0/17:0 triacylglycerol and C17:0 choresteryl ester, 0.2 nmol each, from Avanti Polar Lipids) were added to the samples. Samples were vortexed and sonicated until they appeared dispersed and extracted at 48° C. overnight. The samples were then evaporated and transferred to 1.5 ml Eppendorf tubes after the addition of 0.5 ml of methanol. Samples were evaporated to dryness, and stored at -80° C. until analysis. Before analysis, 150 µl of methanol were add to the samples, centrifuged at 13,000 g for 3 min, and 130 µl of the supernatants was transferred to ultra-performance liquid chromatography (UPLC) vials for injection and analysis.

Sphingolipids: A total of 750 µl of a methanol-chloroform (2:1, vol/vol) solution containing internal standards (N-dodecanoylsphingosine, N-dodecanoylglucosylsphingosine, N-dodecanoylsphingosylphosphorylcholine, C17-dihydrosphingosine, 0.2 nmol each, from Avanti Polar Lipids) were added to 0.5 mg protein lysate. Samples were extracted at 48° C. overnight and cooled. Then, 75 µl of 1 M KOH in methanol was added, and the mixture was incubated for 2 h at 37° C. Following addition of 75 µl of 1 M acetic acid, samples were evaporated to dryness, and stored at -80° C. until analysis. Before analysis, 150 µl of methanol were add to the samples, centrifuged at 13,000 g for 5 min and 130 µl of the supernatant were transferred to a new vial and injected.

Lipids were analyzed by liquid chromatography-high resolution mass spectrometry (LC-HRMS). LC-HRMS analysis was performed using an Acquity ultra high-performance liquid chromatography (UHPLC) system (Waters, USA) connected to a Time of Flight (LCT Premier XE) Detector. Full scan spectra from 50 to 1800 Da were acquired, and individual spectra were summed to produce data points each of 0.2 sec. Mass accuracy at a resolving power of 10,000 and reproducibility were maintained by using an independent reference spray via the LockSpray interference. Lipid extracts were injected onto an Acquity UHPLC BEH C8 column (1.7 µm particle size, 100 mm × 2.1 mm, Waters, Ireland) at a flow rate of 0.3 mL/min and a column temperature of 30° C. The mobile phases were methanol with 2 mM ammonium formate and 0.2% formic acid (A)/water with 2 mM ammonium formate and 0.2% formic acid (B). A linear gradient was programmed as follows : 0.0 min: 20% B; 3 min: 10% B; 6 min: 10% B; 15 min: 1% B; 18 min: 1% B; 20 min: 20% B; 22 min: 20% B.

Positive identification of compounds was based on the accurate mass measurement with an error <5 ppm and its LC retention time, compared with that of a standard (92%).

Quantification was carried out using the extracted ion chromatogram of each compound, using 50 mDa windows. The linear dynamic range was determined by injecting mixtures of internal and natural standards. Since standards for all identified lipids were not available, the amounts of lipids are given as pmol equivalents relative to each specific standard.

Sphingolipids (Cer, ceramide; SM, sphingomyelin; and glycoCer, monohexosylceramide and dihexosylceramide), glycerophospholipids (PC, phosphatidylcholine; PE, phosphatidylethanolamine; and PS, phosphatidylserine), diacylglycerol (DAG), and triacylglycerol (TAG) were annotated using “C followed by the total fatty acyl chain length:total number of unsaturated bonds” and the “lipid subclass” (e.g., C32:2-PC). If the sphingoid base residue was dihydrosphingosine, the name contained a “DH” prefix. Plasmalogens and lysophospholipids were annotated as O- and L.

Separation of glucosylceramides and galactosylceramides was achieved using a Acquity UPLC BEH HILIC column (1.7 µm particle size, 100 mm × 2.1 mm„ Waters) as described by Boutin et al, (Tandem Mass Spectrometry Multiplex Analysis of Glucosylceramide and Galactosylceramide Isoforms in Brain Tissues at Different Stages of Parkinson Disease. Anal. Chem. 2016) with minor modifications. The same LC-HRMS described above was used.

### 4.3. Genomics

Genomics experiments have shown that the genetically modified Mycoplasma bacteria as described herein contain a modified genomic signature that contribute to improved characteristics when said Mycoplasma bacterium is introduced in the host organism. Identical observation can be made for the transcriptome of said modified Mycoplasma bacteria.

### 4.4. Peroxide Production

It could be observed that replacement of GlpD by GpsA (Chassis CV8) abolishes peroxide production (FIG. 6).

### 4.5. Pathogenicity and Infectivity

### 4.5.1. Mammary Gland Model

No histopathological lesions were observed in the mice mammary glands when infected with WT M. pneumoniae and different chassis (see Table 11 below). It could be observed that the CV2 strains had less lesions, while the CV16 strains behaved as a WT Mycoplasma bacterium in term of its haemorrhagic phenotype. Table 11 shows the relative haemorrhagic phenotypes observed for the different strains.

TABLE 11

Microscopic lesions in the mammary glands after infection with M. pneumoniae

| Strain | Leg CFU/mama (media+SD) | Lesiones microscópicas | | | | | | |
|---|---|---|---|---|---|---|---|---|
| | | Proportion de parénquima | Grasa(%) | Adenómeros | | Conduct. Excretores | | Reacción inflamatoria* |
| | | | | PMNs | Secreción | PMNs | Secreción | |
| WT | 7.16±0.98 | 38.3±7.53 | 61.6±7.53 | 0.92±0.38 | 0.92±0.38 | 2.20±0.76 | 0.60±0.22 | 1.58±0.49 |
| CV1 | 5.95±0.68 | 38.0±8.37 | 62.0±8.37 | 0.8±0.45 | 1±0.50 | 1.5±0.79 | 0.5±0 | 1.80±0.27 |
| CV2 | 7.21±0.50 | 43.3±15.06 | 56.6±15.06 | 1.08±0.97 | 0.92±1.07 | 1.95±1.32 | 1.08±0.97 | 1.17±0.88 |
| CV8 | 6.20±0.98 | 47.5±15 | 52.5±15 | 0.75±0.29 | 1.25±0.87 | 1.38±1.03 | 1.25±0.87 | 1.13±0.63 |

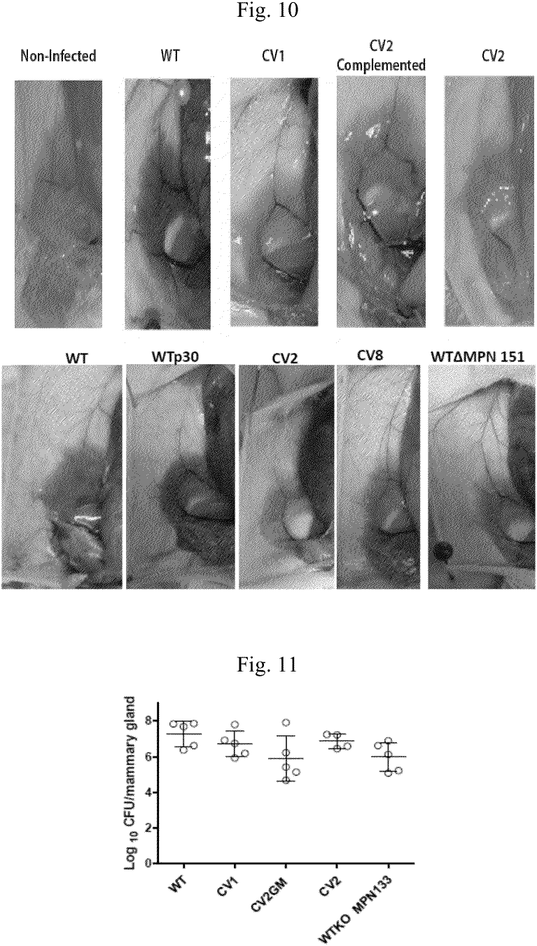

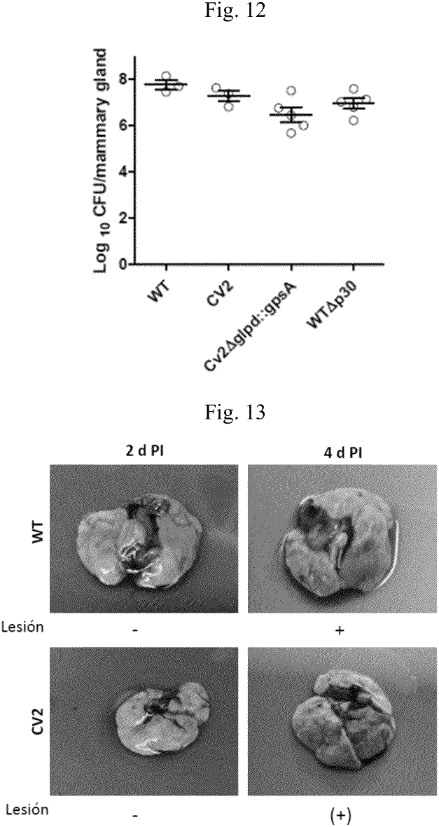

The macroscopic haemorrhagic phenotype for the different chassis haemorrhagic phenotype is depicted in FIG. 10. Persistence of the mammary glands of the strains was also assessed and the results hereof are depicted in FIGS. 11 and 12. Further, the inflammatory response in the mammary glands was also assessed for several strains. Obtained results are depicted in Table 12.

TABLE 12

Inflammatory response in the mammary glands upon infecting with different chassis

| | | | | | |
|---|---|---|---|---|---|
| ILs | Control | WT | CV2 | CV1 | KOMPN133 |
| TNF-A | 0.064±0.042 | 1.530±0.6* | 0.49±0.221$ | 1.579±0.963 | 0.327±0.323$ |
| KC | 0.766±0.514 | 3.889±0.915* | 2.895±1.058* | 1.44±0.715 | 0.411+0.341$ |
| INF-γ | 0.329±0.296 | 0.633±0.142 | 0.462±0.228 | 0.318±0.131 | 0.210±0.228 |
| IL-IB | 0.024±0.029 | 0.454±0.254* | 0.172±0.064 | 0.13±0.125$ | 0.041±0.05$ |
| IL-4 | 0.002±0.002 | 0.003±0.0001 | 0.002±0.002 | 0.004±0.004 | 0.0007±0.0002 |
| IL-6 | 0.035±0.033 | 0.740±-0.558* | 0.295±9.367 | 0.029±0.016 | 0.040±0.041 |
| IL-18 | 0.726±0.497 | 1.632±0.516 | 1.239±1.076 | 0.001+0.001 | 0.003±0.002 |
| MIP-la | 0.238±0.326 | 1.645±1.011 | 0.329±0.053 | 5.173±3.429 | 3.402±2.056 |
| MCP-1 | 0.726±0.497 | 0.031±0.016 | 0.037±0.012 | 5.49±3.33$ | 4.58±3.17$ |
| TLR2 | 0.42±0.213 | 3.087±1.288* | 1.33±0.946* | 2.447±1.00* | 1.84±0.4 |
| IL12p40 | 0.036±0.046 | 0.031±0.016 | 0.0375±0.012 | 0.060±0.035 | 0.072±0.070 |
| IL23 | 0.581±0.522 | 6.913±3.546 | 4.416±3.543 | 0.0131±0.06 | 0.079±0.08 |
| *p<0.05 Strains vs Control | | | | | |
| $ p<0.05 WT v Strains | | | | | |

A significant response is observed for KC (IL8) in all cases except for WT KO MPN133 (corresponding to CV31). Similar results were obtained for the TRL2 receptor. The response to the other factors is variable depending of the Mycoplasma strain and does not appear to show a consistent pattern.

### 4.5.2. Lung Model

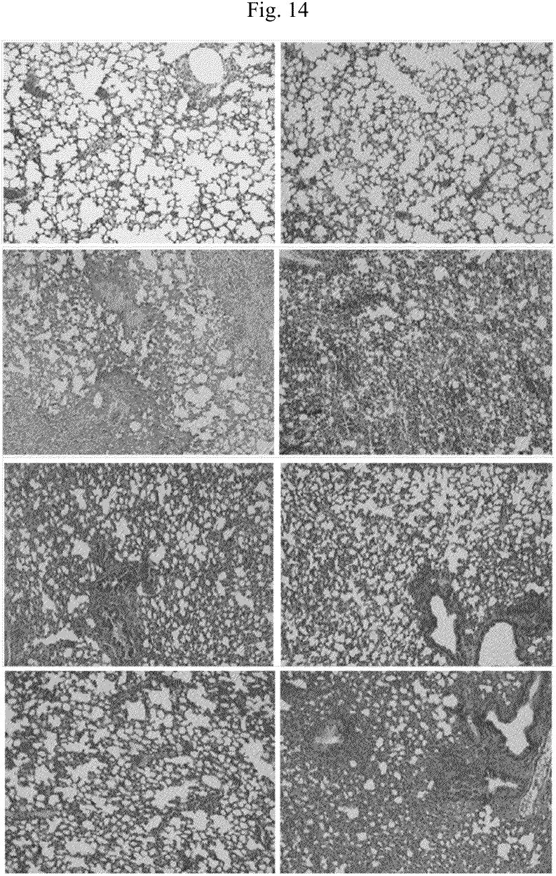

Histopathologic analysis of the mice CD1 lungs infected with the WT, CV2 and CV8 chassis was performed and the obtained results are depicted in FIGS. 13, 14, and 15. The double blind analysis shows that the CV8 chassis does not display pathogenicity, which is in accordance with the control condition wherein the lungs were inoculated with a volume of PBS equal to the the inoculum containing the bacteria. The results further show that the WT is pathogenic, while the CV2 chassis has an intermediate phenotype.

To verify whether deletion of some of the proteins hypothesized to be involved in pathogenesis or any other undesired trait of M. pneumoniae have any effect on the survival in the lung of infected animals, said animals were sacrificed 2 and 4 days post infection, wherein infection occurred with approximately 10⁷ bacterial cells. A lung extract was prepared and plated on petri dishes with appropriate antibiotics. Subsequently the number of colonies (CFUs) were counted.

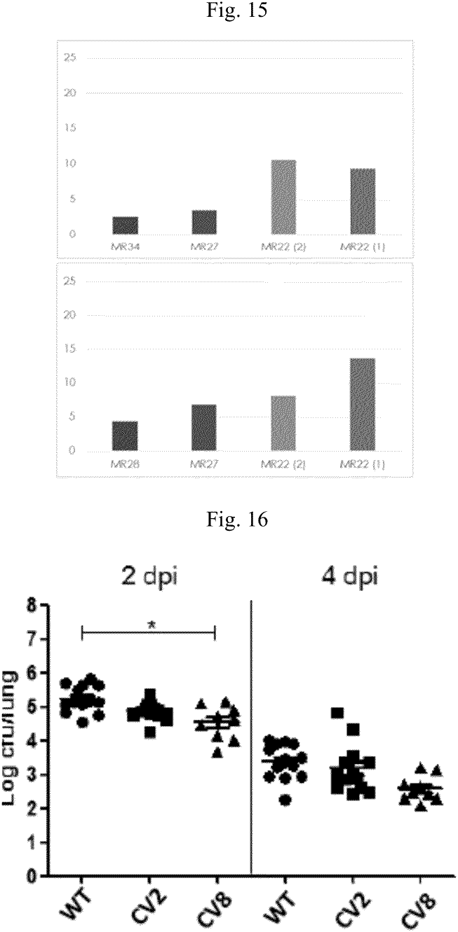

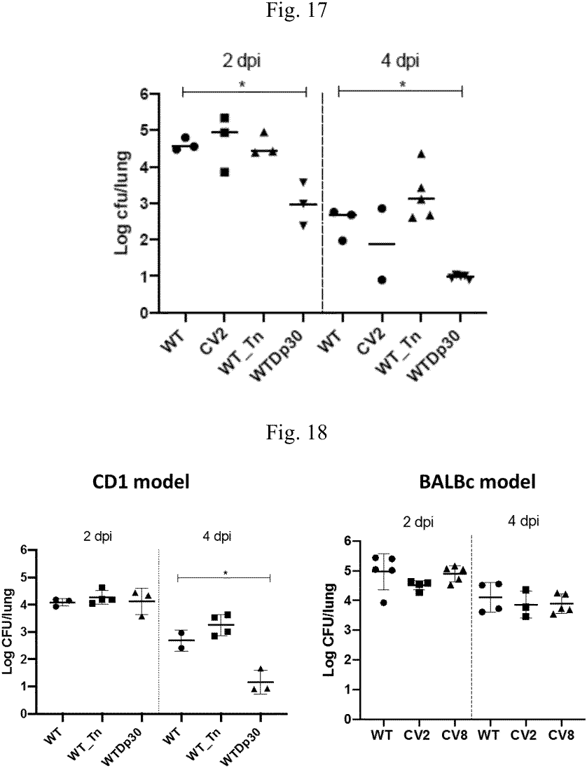

Animals were infected with 1.4 × 107 CFUs of the WT strain and 7.8×106 of the CV2 chassis (FIG. 16). No significant differences in bacterial load at days 2 and 4 could be observed when comparing the WT, CV2 and CV8 strains. This experiment was repeated with different Mycoplasma strains as depicted in FIG. 17. Here, it could be observed that the CV16 strain which lacks the adhesion protein p30 (mpn453) is washed (i.e. cleared) at a faster rate than the WT, CV2 and control CV16 strains; suggesting that attachment in the lung is necessary for the chassis to persist in the lung of the infected animal.

Additionally, CD1 mice were infected with approximately 10⁷ CFUs to monitor the inflammatory response. The induction of different markers of inflammation are shown in Table 13.

TABLE 13

Arbitrary units of different inflammatory markers. CON indicates the control condition. Top panel is indicative for a first repeat, bottom panel is indicative for a second repeat

| | | 2 dpi | | |
|---|---|---|---|---|
| | CON | WT | CV2 | CV8 |
| TNF-a | 0.005 ± 0.002 | 0.081 ± 0.037 * | 0.02 ± 0.010 | 0.080 ± 0.054 * |
| KC | 0.128 ± 0.097 | 2.313 ± 1.063 * | 1.522 ± 0.614 *, $ | 1.452 ± 0.439 *, $ |
| INF-γ | 0.016 ± 0.001 | 0.105 ± 0.051 * | 0.036 ± 0.025 $ | 0.029 ± 0.003 $ |
| MIP-1a | 0.228 ± 0.050 | 1.061 ± 0.419 * | 0.508 ± 0.089 $ | 0.120 ± 0.081 $ |
| MCP-1 | 0.062 ± 0.033 | 0.510 ± 0.205 * | 0.281 ± 0.130 | 0.393 ± 0.180 * |
| IL-6 | 0.015 ± 0.007 | 0.036 ± 0.011 * | 0.026 ± 0.008 | 0.060 ± 0.009 * |
| | | 2 dpi | | |
| | CON | WT | CV2 | CV8 |
| TN F-a | 0.09 ± 0.06 | 0.325 ± 0.237* | 0.135 ±0.05 | 0.22 ± 0.03 |
| KC | 0.10 ± 0.07 | 1.32 ± 0.99* | 0.39 ± 0.09 *, $ | 1.15 ± 0.42 * |
| INF-γ | *0.016 ± 0.001 | 0.21 ± 0.13* | 0.06 ±0.03 $ | 0.32 ± 0.20 $ |
| MIP-1a | 0.17 ± 0.04 | 1.0± 0.69* | 0.20 ± 0.02 $ | 0.47 ± 0.17 $ |
| MCP-1 | 0.66 ± 0.09 | 2.86 ± 1.51* | 0.67 ± 0.23 | 2.21 ± 0.94* |
| IL-6 | 0.006 ± 0.001 | 0.02 ± 0.01 | 0.01±0.00 | 0.02 ± 0.01 |

A mild inflammatory response could be observed in the CD1 mice, the main consistent result being a significant increase in KC (IL-8) in WT, CV2 and CV8 strains, and a more pronounced response for MIP-la when using WT Mycoplasma. The experiment was repeated using 10⁷ and 10⁹ CFUs and measuring cytokine induction by means of qPCR (Table 14).

TABLE 14

Arbitrary qPCR units of different inflammatory markers in CD1 lung mice when infected with 10⁷ and 10⁹ CFUs of the WT chassis (MR30)

| | | 2 dpi | | Fold change |
|---|---|---|---|---|
| | CO N | 10^{∧}7 | 10^{∧}9 | (10^{∧}9/10^{ʌ}7) |
| TNF-a | 0.005 ± 0.002 | 0.081 ± 0.037 * | 5.53 ± 3.6 *, $ | 66.05 |
| KC | 0.128 ± 0.097 | 2.313 ± 1.063 * | 17.55 ± 11.94 *, $ | 7.59 |
| INF-γ | 0.016 ± 0.001 | 0.105 ± 0.051 * | 106.4 ± 64.28*, $ | 1013.33 |
| MIP-1a | 0.228 ± 0.050 | 1.061 ± 0.419 * | 733 ± 364 *, $ | 690.86 |
| MCP-1 | 0.062 ± 0.033 | 0.510 ± 0.205 * | 0.619 ± 0.376* | No change |
| IL-1b | 0.084 ± 0.038 | 0.105 ± 0.043 | 2.49 ± 1.93 *, $ | 23.71 |
| IL-4 | 0.005 ± 0.001 | 0.006 ± 0.003 | 0.003 ± 0.001 | No change |
| IL-6 | 0.015 ± 0.007 | 0.036 ± 0.011 * | 0.82 ± 0.9 *, $ | 22.78 |
| IL-12 p40 | 0.16 ± 0.007 | 0.032 ± 0.007 | 0.38 ± 0.1 *, $ | 11.88 |
| TLR2 | 0.222 ± 0.070 | 0.321 ± 0.112 | 75.97 ± 21.47*, $ | 236.67 |

When repeating the infection with the WT at 10⁷ CFUs, in accordance to Table 13, a significant signal for KC (IL-8), MIPla and MCP1 could be observed. By increasing the dose of bacteria to 10^9 a very significant response for all markers could be observed except for IL-4 and IL-12. Intriguingly, increasing the dose does not impart a change in MCP-1 expression levels.

### 4.6. In Vivo Replication and Maintenance

In order to assess the replication rate in vivo of the WT and CV8 Mycoplasma strains, a time course experiment was conducted wherein the different Mycoplasma strains were equipped with a suicide vector (respectively WT_vec and CV8_vec). Sampling of the Mycoplasma populations was performed at different time points. If Mycoplasma bacteria replicated in the intermediary time point, the amount of genomic DNA (gDNA) increased relative to the suicide vector DNA. qPCR was used to quantify the Mycoplasma gDNA and suicide vector DNA. After an initial proof of concept test in vitro, it could be shown that the Mycoplasma gDNA: suicide vector ratio increased over time in lung tissue, bronchoalveolar lavage (BALF), and serum of the test mice infected with 10⁸ CFU. The obtained results for BALF are depicted in FIG. 25.

The clearance rate for the WT_vec and CV8_vec strains, calculated as the decrease of CFUs at 24h, was very similar for both strains, 66.6% and 63.6% for WT_vec and CV8_vec samples respectively (considering mean values of CFUs extrapolated from the Ct values using as standard the CFUs plated in the experiment). At 6 h the averaged ratios of vector/cells were 0.91 and 1.36 for WT_vec and CV8_vec samples, respectively. At 24 h, those values decreased to 0.3 and 0.28. This implies that for the WT_vec there was a reduction of 3 times in the number of vectors per cell whereas for CV8_vec this reduction was between 4-5 times. In the first round of replication, the reduction of the ratio should be half and in the next 4 times. It implies that the WT_vec performed approximately 1′5-2 rounds of replication and CV8_vec did 2-2.5 rounds in 18 h. It represents an in vivo doubling time of 9-12h and 7-9 h for WT_vec and CV8_vec cells, respectively. The minor differences observed between each strain may be an artefact of the in vivo assay as depicted by the error bar for the CV8_vec at 6 h.

### 4.7. Dissemination of Mycoplasma to Other Tissues

A further experiment was conducted to assess whether M. pneumoniae bacteria spread to other tissues after initial administration to the lung. Mice were inoculated with a dose of 10⁶ wild type Mycoplasma bacteria or as negative control with PBS. After 6 and 48 hours, blood, liver, spleen, kidney, and trachea were assessed for the presence of Mycoplasma. Mycoplasma could be detected in the trachea, lungs, and BALF of the subject animals (Table 15). Mycoplasma was not detected in blood, liver, spleen, or the kidneys of the infected animals at any time point. Hence, it can be concluded that Mycoplasma infection does not spread to other tissues.

TABLE 15

Wild type Mycoplasma detection by plating on HF plates in distinct samples derived from infected animals (infection dose: 10⁶)

| | | | | | | |
|---|---|---|---|---|---|---|
| Mice | Group | Blood | Liver | Spleen | Kidney | Trachea |
| R1 | WT vec 6 h | - | - | - | - | + |
| R2 | WT vec 6 h | - | - | - | - | + |
| R3 | WT vec 6 h | - | - | - | - | + |
| R4 | WT vec 6 h | - | - | - | - | + |
| R5 | PBS 6 h | - | - | - | - | - |
| R6 | PBS 6 h | - | - | - | - | - |
| R7 | WT vec 48 h | - | - | - | - | + |
| R8 | WT vec 48 h | - | - | - | - | + |
| R9 | WT vec 48 h | - | - | - | - | - |
| R10 | WT vec 48 h | - | - | - | - | + |
| R11 | WT vec 48 h | - | - | - | - | + |
| R12 | PBS 48 h | - | - | - | - | - |
| R13 | PBS 48 h | - | - | - | - | - |

A second experiment was conducted with a higher infection dose (10⁸) of wild type, CV2, or CV8 bacteria. In accordance with the first experiment, Mycoplasma could only be detected in lung tissue, BAL, and the trachea (Table 16). This observation could be made for any tested bacterial strain.

TABLE 16

Mycoplasma detection by plating on HF plates in distinct samples derived from infected animals (infection dose: 10⁸)

| | | | Log₁₀ CFU/Lung or BAL: | | Presence or absence of Mycoplasma in organs: | | | | |
|---|---|---|---|---|---|---|---|---|---|
| Mice | Strain | Time point | Lung | BAL | Blood | Liver | Spleen | Kidney | Trachea |
| R1 | WT | 6 h | 7.46 | 6.60 | - | - | - | - | + |
| R2 | | | 8.20 | 6.63 | - | - | - | - | + |
| R3 | | | 8.01 | 7.55 | - | - | - | - | + |
| R4 | | | 7.92 | 8.82 | - | - | - | - | + |
| R5 | CV8 | 6 h | 8.22 | 6.71 | - | - | - | - | + |
| R6 | | | 7.69 | 5.53 | - | - | - | - | + |
| R7 | | | 8.11 | 5.28 | - | - | - | - | + |
| R8 | | | 8.14 | 5.58 | - | - | - | - | + |
| R9 | PBS | 6 h | 0.94 | 0.18 | - | - | - | - | - |
| R10 | | | 1.13 | 0.18 | - | - | - | - | - |
| R11 | WT | 24 h | 7.37 | 4.98 | - | - | - | - | + |
| R12 | | | 7.88 | 5.48 | - | - | - | - | + |
| R13 | | | 7.68 | 5.43 | - | - | - | - | + |
| R14 | | | 7.38 | 5.58 | - | - | - | - | + |
| R15 | | | 7.46 | 5.49 | - | - | - | - | + |
| R16 | CV8 | 24 hh | 7.21 | 4.97 | - | - | - | - | + |
| R17 | | | 7.55 | 6.18 | - | - | - | - | + |
| R18 | | | 7.41 | 5.22 | - | - | - | - | + |
| R19 | | | 7.48 | 5.62 | - | - | - | - | + |
| R20 | | | 7.17 | 4.66 | - | - | - | - | + |
| R21 | PBS | 24 h | 1.02 | 0.18 | - | - | - | - | - |
| R22 | | | 1.10 | 0.18 | - | - | - | - | - |

### 4.8. Confirmation of Results in BALBc Mice.

A second strain of mice (BALBc) was used to confirm the results. BALBc mice have been reported to display exacerbated immune responses following M. pneumoniae infection. The WT, CV2 and CV8 strains were used to repeat the above described lung infection experiments. Interestingly, it could be observed that all strains persist better in BALBc mice when compared to CD1 mice (FIG. 18), suggesting that in this mouse model Mycoplasma is maintained longer and may hence be a better model to study immune responses caused by Mycoplasma infections.

### 5. Rational Design of M. pneumoniae Strains Minimizing or Avoiding the Risk of Guillain-Barre Syndrome Development by the Recipient Host

MPN257 and MPN483 are classified as important fitness genes (Lluch et al, Molecular Systems Biology, 2015). Nevertheless, deletion of each gene individually was feasible (data not shown). However, these deletions compromise growth of M. pneumoniae in vivo (FIG. 20). Deletion of MPN483 has a dramatic effect in growth rate (FIG. 20), while this effect is smaller in the strain wherein MPN257 is deleted.

Hence, it was decided to replace MPN483 by other processive glycosil transferases that preferentially use UDP-glucose instead of UDP-galactose: MG_517 (MG-517), MAGA RS00300 or ugtP, to improve growth rate and prevent formation of galactosyl-cerebrosides and galactosyl-diacylglycerol. Mass spectroscopy analysis shows that the three proteins are expressed in M. pneumoniae (see Table 17). Each of the proteins are expressed at expression levels higher than the MPN483 expression level.

TABLE 17

Mass spectroscopy analysis of the different mutant strains and WT M. pneumoniae. Values indicate the area under the curve for the average of the three peptides with higher area under the curve values as commonly done in mass spectroscopy analysis

| Strain | MPN483 | MG_517 | MAGA_RS00300 | UgtP |
|---|---|---|---|---|
| WT | 5.22E7+/-2.5E6 | 0.00 | 0.00 | 0.00 |
| MPN483 KO | 0.00 | 0.00 | 0.00 | 0.00 |
| MG-517 | 0.00 | 3.3E9+/-2.2E8 | 0.00 | 0.00 |
| MAGA RS00300 | 0.00 | 0.00 | 2.1E9+/-3.2E8 | 0.00 |
| UgtP | 0.00 | 0.00 | 0.00 | 4.8E8+/-4E7 |

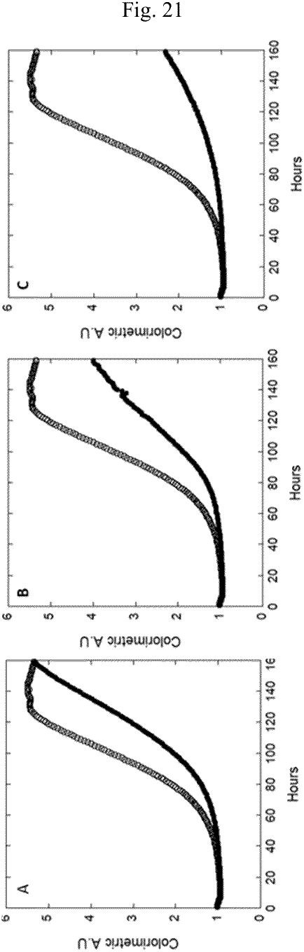

Replacement of MPN 483 by either MG_517 or MAGA_RS00300 significantly improved the growth with respect to the deletion of MPN483 (FIGS. 21A, B), while the improvement by inserting ugtP was limited and only a slight improvement in growth could be observed when compared to the MPN483 knock-out strain (FIG. 21C).

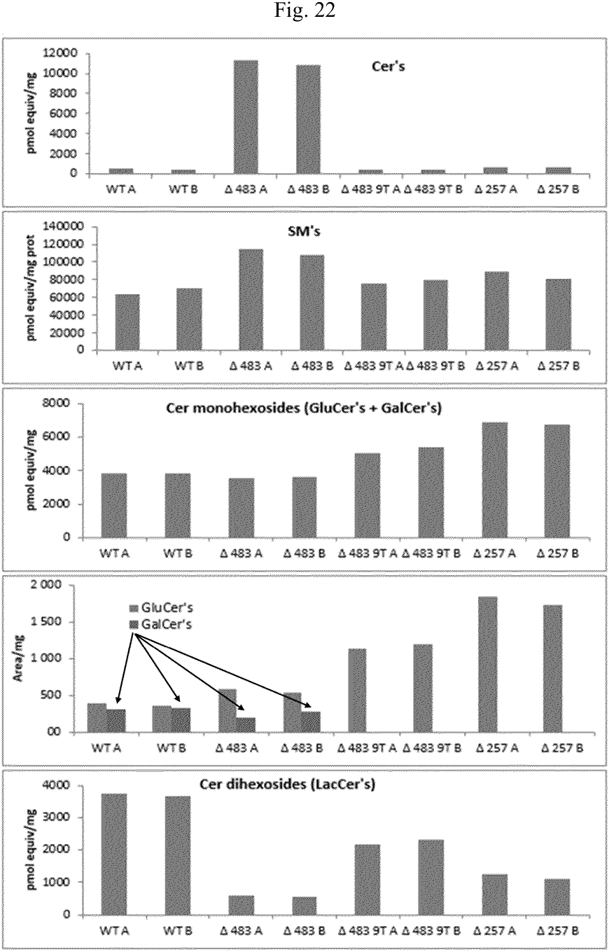

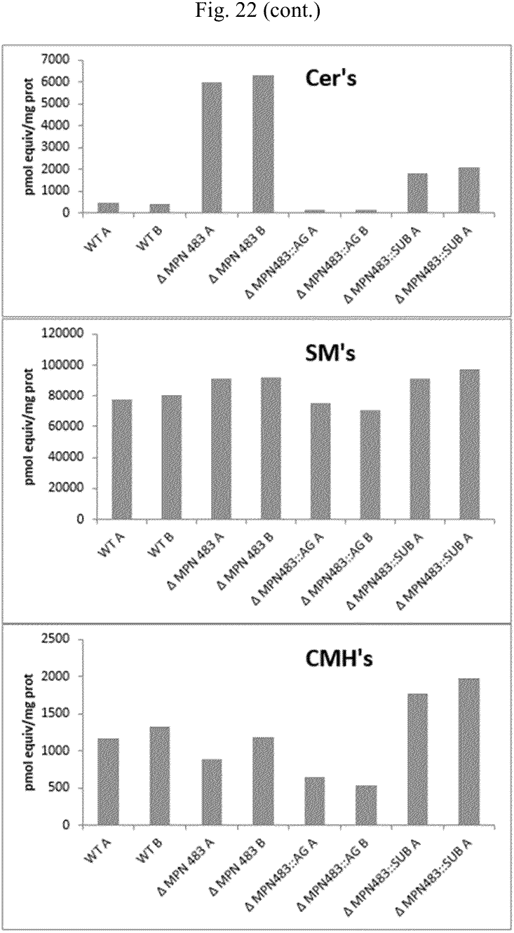

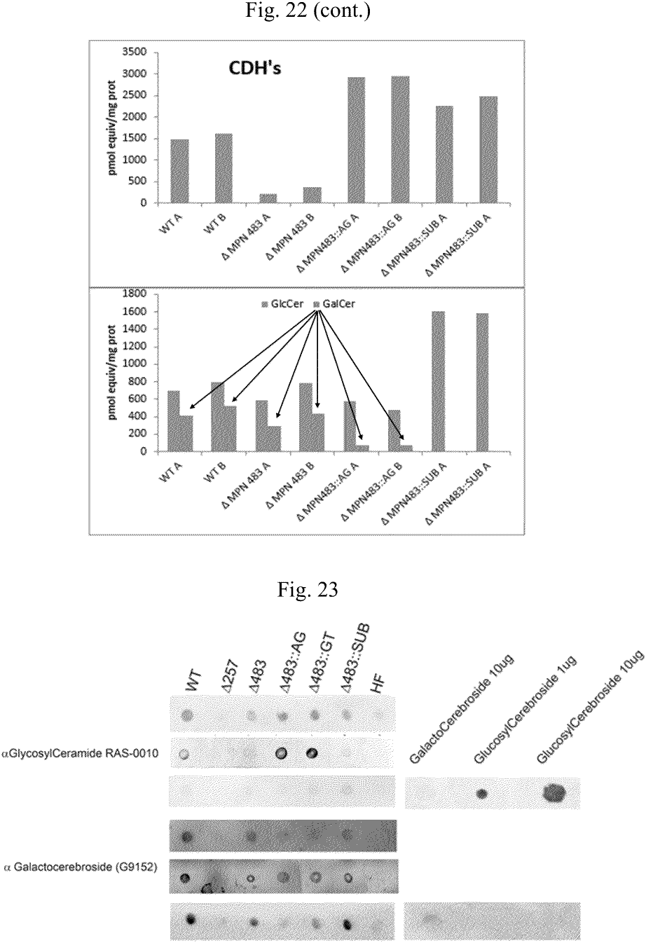

Further mass spectrometry analysis confirmed the hypothesis that deletion of MPN483 leads to an accumulation of toxic ceramides in M. pneumoniae which can be considered a feasible cause for the slow growth (FIG. 22). Ceramide accumulation was improved in the ugtP strain and there was no ceramide accumulation in the MPN257 KO strains or in the MG_517 and MAGA_RS00300 strains which each show an improved growth when compared to MPN483 KO or ugtP strains. This indicates that MPN483 like MG_517 and MAGA_RS00300 are mainly responsible for the formation of cerebrosides in M. pneumoniae. FIG. 22C shows the total concentration of monohexoside-ceramides, FIG. 22D the total concentration of dihexoside-ceramides. The levels of monohexoside-ceramides and dihexoside-ceramides are anticorrelated for the MG_517, MAGA_RS00300 and KO MPN257 in comparison with the WT. In MG_517 and MPN257 KO strains more monohexoside and less dihexoside-ceramides are present when compared to the WT, in contrast with the MAGA_RS00300 strain. The ugtP strain contains more dihexoside-ceramides when compared to the WT strain, while the content for monohexoside-ceramides is much higher. Only the WT and MPN483 KO strains contain considerable amounts of mono-galactocerebroside. In contrast, the MAGA_RS00300 strain contains about 8-10 fold less mono-galactocerebrosides, while the MG_517 and the ugtP did not contained mono-galactocerebrosides. For the MPN257 KO, MG_517 and ugtP trains a higher concentration of mono-glucorebrosides could be observed.

By mass spectroscopy it is not possible to distinguish the different possible dihexoside-cerebrosides (Glu-Glu; Gal-Gal; Glu-Gal and Gal-Glu). This means that although no galactocerebroside in MG_517, KO MPN257 and ugtP could be detected, and very low values in the MAGA_RS00300 strain were detected, we could have galactose present in the dihexoside-cerebrosides. To elucidate whether this is indeed the case commercial antibodies against galactocerebrosides (Merck #G9152) and glucosylceramide (RAS-0010 glycobiotech) were used in a final dot blot experiment were used (FIG. 23). For the WT, MPN483 KO, and UgtP strains a clear positive signal could be observed. The signal is reduced in MG_517, almost disappears in MAGA RS00300, and is non-existent in the MPN257 KO. Indeed, KO of MPN257 completely eliminates any cerebroside with galactose and hence confirms that there is no other enzyme with a similar activity. The dot blot also corroborates the presence of mono-galatocerebroside in the KO of MPN483 and therefore supports the idea that MPN025 and MPN078 could be involved in the formation of monohexose-cerebrosides, but not dihexose-cererbosides which will be exclusively produced by MPN483. In the case of the MG_517 and ugtP strains the findings suggest that although there is no mono-galatocerebroside there could be dihexose-cerebrosides having galactose bound to glucosecerebroside. This does not seem apparent in the MAGA_RS00300 strains since the very weak signal that may be observed in the western blot corresponds to the small amount of mono-galactocerebroside and not with the presence of dihexose-cerebrosides having galactose.

Hence in conclusion, a favorable M. pneumoniae strain having minimal chances of provoking Guillain-Barre syndrome in a recipient and growing well does not comprise a functional MPN257 gene nor a functional MPN483 gene, and is a M. pneumoniae wherein the function of the MPN483 gene is replaced by any glycosyltransferases that uses UDP-glucose and not UDP galactose, for example with either MAGA RS00300, MG_517, or a combination thereof.

### 6. Funding Acknowledgement

The projects leading to this application have received funding from the European Union’s Horizon 2020 research and innovation programme and from the European Research Council (ERC) under grant agreements No 634942 (MycoSynVac) and No 670216 (MycoChassis), respectively.

1. A genetically modified Mycoplasma bacterium, wherein the Mycoplasma bacterium comprises in its genome a deletion, substitution, and/or insertion of one or more nucleotides in the operon of the Ca2+ dependent cytotoxic nuclease gene (MPN133) and the operon of the ADP-ribosyltransferase CARDS gene (MPN372) that reduce the pathogenicity and/or immunogenicity of the Mycoplasma bacterium as compared to a reference Mycoplasma bacterium having an otherwise identical genomic sequence lacking the deletion, substitution, and/or insertion in the operons.

2. The genetically modified Mycoplasma bacterium of claim 1, wherein the bacterium further comprises a deletion, substitution, and/or insertion of one or more nucleotides in a gene or operon encoding a peroxide producing protein.

3. The genetically modified Mycoplasma bacterium of claim 1, wherein the bacterium further comprises a deletion, substitution, and/or insertion of one or more nucleotides in one or more genes or operons encoding a protein capable of eliciting Guillain-Barre in a host organism.

4. The genetically modified Mycoplasma bacterium of claim 1, wherein the Mycoplasma bacterium is selected from the group consisting of: M. adleri, M. agalactiae, M. agassizii, M. alkalescens, M. alligatoris, M. alvi, M. amphoriforme, M. anatis, M. anseris, M. arginine, M. arthritidis, M. auris, M. bovigenitalium, M. bovirhinis, M. bovis, M. bovoculi, M. buccale, M. buteonis, M. californicum, M. canadense, M. canis, M. capricolum, M. capricolum subsp. capricolum, M. capricolum subsp. capripneumoniae, M. caviae, M. cavipharyngis, M. ciconiae, M. citelli, M. cloacale, M. collis, M. columbinasale, M. columbinum, M. columborale, M. conjunctivae, M. corogypsi, M. cottewii, M. cricetuli, M. crocodyli, M. cynos, M. dispar, M. edwardii, M. elephantis, M. equigenitalium, M. equirhinis, M. falconis, M. fastidiosum, M. faucium, M. felifaucium, M. feliminutum, M. felis, M. feriruminatoris, M. fermentans, M. flocculare, M. gallinaceum, M. gallinarum, M. gallisepticum, M. gallopavonis, M. gateae, M. genitalium, M. glycophilum, M. gypis, M. haemocanis, M. haemofelis, M. haemomuris, M. hominis, M. hyopharyngis, M. hyopneumoniae, M. hyorhinis, M. hyosynoviae, M. iguana, M. imitans, M. indiense, M. iners, M. iowae, M. lagogenitalium, M. leachii, M. leonicaptivi, M. leopharyngis, M. lipofaciens, M. lipophilum, M. maculosum, M. meleagridis, M. microti, M. moatsii, M. mobile, M. molare, M. mucosicanis, M. muris, M. mustelae, M. mycoides, M. mycoides subsp. capri, M. mycoides subsp. mycoides, M. neophronis, M. neurolyticum, M. opalescens, M. orale, M. ovipneumoniae, M. ovis, M. oxoniensis, M. penetrans, M. phocicerebrale, M. phocidae, M. phocirhinis, M. pirum, M. pneumoniae, M. primatum, M. pullorum, M. pulmonis, M. putrefaciens, M. salivarium, M. simbae, M. spermatophilum, M. spumans, M. sturni, M. sualvi, M. subdolum, M. suis, M. synoviae, M. testudineum, M. testudinis, M. tullyi, M. verecundum, M. wenyonii, M. yeatsii, and M. coccoides, preferably wherein said Mycoplasma bacterium is selected from the group consisting of: M. pneumoniae, M. genitalium, M. hyorhinis, M. bovis, M. agalactiae, M. gallisepticum, and M. feriruminatoris.

5. The genetically modified Mycoplasma bacterium of claim 1, wherein said Mycoplasma bacterium is a M. pneumoniae bacterium or M. pneumoniae M129-B7.

6. The genetically modified Mycoplasma bacterium of claim 1, wherein the bacterium further comprises a deletion, substitution, and/or insertion of one or more nucleotides in a gene or operon encoding:
a second nuclease, a surface nuclease, membrane nuclease A (MPN491), a cytoadherence protein, MPN141, MPN142, MPN453, MPN447, MPN309, MPN310, MPN452, an immunogenic protein that is capable of eliciting an immune response in a host organism, conserved hypothetical protein MPN_400 (MPN400), a protein that inhibits growth of said bacterium in a bioreactor, a chaperone protein YajL (MPN294), a prolipoprotein diacylglyceryl transferase and a prolipoprotein signal peptidase, MPN224 and MPN293, an oncogenic protein, high affinity transport system protein p37 (MPN415), a secreted Mycoplasma gene product, MPN036, MPN592, MPN509, MPN647, MPN084, MPN625, MPN213, MPN489, MPN444, MPN642, MPN398, MPN083, a lipoprotein, MPN152, MPN162, MPN199, MPN200, MPN233, MPN271, MPN284, MPN288, MPN333, MPN372, MPN597, MPN602, MPN611, MPN011, MPN052, MPN054, MPN058, MPN097, MPN098, MPN363, MPN369, MPN408, MPN411, MPN436, MPN439, MPN442, MPN456, MPN467, MPN506, MPN523, MPN582, MPN585, MPN586, MPN587, MPN588, MPN590, MPN591, MPN639, MPN640, MPN641, MPN643, MPN644, MPN645, MPN646, MPN648, MPN649, MPN650, or MPN654.

7-14. (canceled)

15. The genetically modified Mycoplasma bacterium of claim 1, wherein the reduced pathogenicity and/or immunogenicity is a reduction of toxicity by at least 30%, when introduced into the respiratory system of a host organism, as compared to the reference Mycoplasma bacterium.

16. (canceled)

17. The genetically modified Mycoplasma bacterium of claim 1, wherein the Mycoplasma bacterium further comprises a nucleotide sequence encoding an exogenous gene product or a functional fragment thereof.

18. The genetically modified Mycoplasma bacterium of claim 17, wherein the exogenous gene product or functional fragment thereof is a protein, a therapeutic protein, a protein involved in specific attachment to a host protein, an enzyme, immunogenic protein, or a DNA-binding protein.

19. (canceled)

20. (canceled)

21. The genetically modified Mycoplasma bacterium of claim 31, wherein the pharmaceutical composition is a medicament.

22. The genetically modified Mycoplasma bacterium of claim 31, wherein the pharmaceutical composition is a vaccine.

23. The genetically modified Mycoplasma bacterium of claim 22, wherein the Mycoplasma bacterium displays at least one distinct exogenous proteogenic sequence on its surface.

24. The genetically modified Mycoplasma bacterium of claim 23, wherein the at least one exogenous proteogenic sequence is an exogenous antigenic sequence.

25. A method of modulating the composition of a lung microbiome in a subject, the method comprising administering to the respiratory system of the subject the genetically modified Mycoplasma bacterium of claim 1.

26. A method of producing an attenuated Mycoplasma bacterium, wherein the method comprises introducing a deletion, substitution, and/or insertion of one or more nucleotides in at least two genes or operons encoding a gene product independently selected from the group consisting of: cytoadherence proteins, lipid synthesis enzymes producing immunogenic products, oxidoreductases, nucleases, toxins, lipoproteins, inflammatory regulating proteins, immunogenic proteins, and cancer inducing proteins.

27. The method according to claim 26, wherein the deletions, substitutions, and/or insertions of one or more nucleotides are introduced in a live Mycoplasma bacterium via a site-directed recombinase, via random transposon insertion, and/or via a site-directed nuclease.

28. The method according to claim 26, wherein the method further comprises transferring the a synthetic genome or portion thereof to a naturally occurring Mycoplasma bacterium.

29. The method according to claim 28, wherein the method further comprises inactivating, degrading, and/or removing the original genome of the live Mycoplasma bacterium.

30. (canceled)

31. The genetically modified Mycoplasma bacterium of claim 1, wherein the Mycoplasma bacterium is comprised in a pharmaceutical composition .

32. The genetically modified Mycoplasma bacterium of claim 1, further comprising a deletion, substitution, and/or insertions of one or more nucleotides in at least one gene or operon selected from the group consisting the genes and operons of Table 1.

33. The genetically modified Mycoplasma bacterium of claim 2, wherein the gene or operon encoding a peroxidase producing protein is a gene or operon encoding glycerol-3-phospate dehydrogenase (MPN051).

34. The genetically modified Mycoplasma bacterium of claim 3, wherein the one or more genes or operons encoding a protein capable of eliciting Guillain-Barre in a host organism is one or more genes or operons encoding a UDP-glucose 4-epimerase (MPN257), and/or a glycosyltransferase (MPN483).
